(19) United States
(12) Patent Application Publication
(10) Pub. No.: US 2025/0127036 A1
(43) Pub. Date: Apr. 17, 2025

(54) ORGANIC COMPOUND AND ORGANIC LIGHT-EMITTING DEVICE

(71) Applicant: CANON KABUSHIKI KAISHA, Tokyo (JP)

(72) Inventors: HIROKAZU MIYASHITA, Miyagi (JP); NAOKI YAMADA, Tokyo (JP); YOSUKE NISHIDE, Kanagawa (JP); HIROKI OHRUI, Tokyo (JP); HIRONOBU IWAWAKI, Kanagawa (JP); TAKAYUKI HORIUCHI, Kanagawa (JP)

(21) Appl. No.: 18/969,021

(22) Filed: Dec. 4, 2024

Related U.S. Application Data
(63) Continuation of application No. PCT/JP2023/018680, filed on May 19, 2023.

(30) Foreign Application Priority Data
Jun. 6, 2022 (JP) 2022-091509

(51) Int. Cl. H10K 85/60; C07D 333/76; C07D 491/048; C07D 495/04; C09K 11/02; C09K 11/06; H10K 50/12; H10K 85/30; H10K 101/30

(52) U.S. Cl. CPC H10K 85/622; C07D 333/76; C07D 491/048; C07D 495/04; C09K 11/02; C09K 11/06; H10K 85/342; H10K 85/624; H10K 85/626; H10K 85/654; H10K 85/657; H10K 85/6572; H10K 85/6574; H10K 85/6576; C09K 2211/1007; C09K 2211/1011; C09K 2211/1029; C09K 2211/1088; C09K 2211/185; H10K 50/12; H10K 2101/30

(57) ABSTRACT

An organic compound represented by the following general formula [1]:

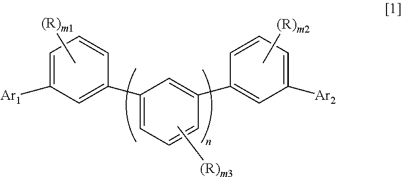

In the general formula [1], Ar₁ and Ar₂ are each independently selected from an aryl group and a heterocyclic group. Ar₁ and Ar₂ are represented by different skeletons. When Ar₁ and Ar₂ have a dibenzothiophene skeleton or a dibenzofuran skeleton, the organic compound has at least one substituent. The substituent represented by R is each independently selected from a deuterium atom, a halogen atom, an alkyl group, an alkoxy group, an amino group, an aryloxy group, an aryl group, a heterocyclic group, a silyl group, and a cyano group. When a plurality of Rs are present, the Rs may be the same or different. n denotes an integer in the range of 2 to 5, and m₁ to m₃ each denote an integer in the range of 0 to 4.

### CROSS-REFERENCE TO RELATED APPLICATIONS

This application is a Continuation of International Patent Application No. PCT/JP2023/018680, filed May 19, 2023, which claims the benefit of Japanese Patent Application No. 2022-091509, filed Jun. 6, 2022, both of which are hereby incorporated by reference herein in their entirety.

### TECHNICAL FIELD

The present invention relates to an organic compound and an organic light-emitting device including the organic compound.

### BACKGROUND ART

An organic light-emitting device (hereinafter sometimes referred to as an “organic electroluminescent device” or an “organic EL device”) is an electronic device that includes a pair of electrodes and an organic compound layer between the electrodes. Electrons and holes are injected from the pair of electrodes to generate an exciton of a light-emitting organic compound in the organic compound layer. When the exciton returns to its ground state, the organic light-emitting device emits light.

With recent significant advances in organic light-emitting devices, it is possible to realize low drive voltage, various emission wavelengths, high-speed responsivity, and thin and lightweight light-emitting devices.

Compounds suitable for organic light-emitting devices have been actively developed. This is because development of a compound with long device life is important to provide a high-performance organic light-emitting device.

As a compound developed so far, Patent Literature 1 discloses the following compound 1-A as an example of a compound with a fused polycyclic group bonded to a phenylene chain. Patent Literature 2 discloses the following compound 1-B. Patent Literature 3 discloses the following compound 1-C. Patent Literature 4 discloses the following compound 1-D.

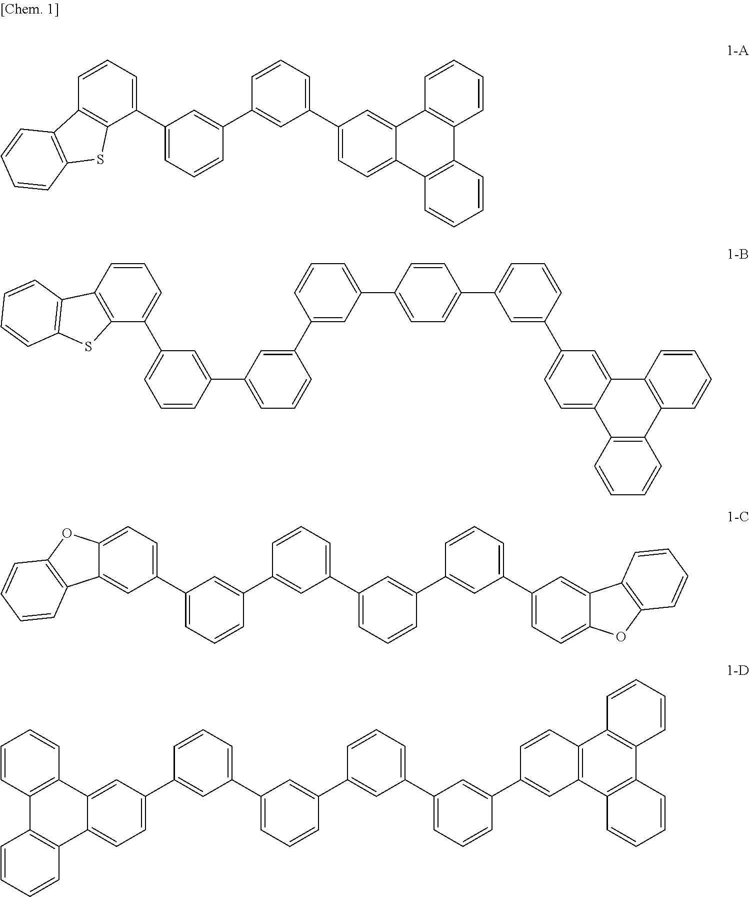

In any of the above compounds, molecules are likely to aggregate in a film, and organic light-emitting devices including these compounds have an device life problem.

### PATENT LITERATURE

PTL 1 PCT Japanese Translation Patent Publication No. WO 2009/021126
PTL 2 PCT Japanese Translation Patent Publication No. WO 2012/133644
PTL 3 PCT Japanese Translation Patent Publication No. WO 2012/153780
PTL 4 PCT Japanese Translation Patent Publication No. WO 2012/050008

### SUMMARY OF INVENTION

The present invention has been made in view of the above problems and aims to provide an organic compound with molecules that are less likely to aggregate in a film.

### CITATION LIST

An organic compound according to the present invention is represented by the general formula [1]:

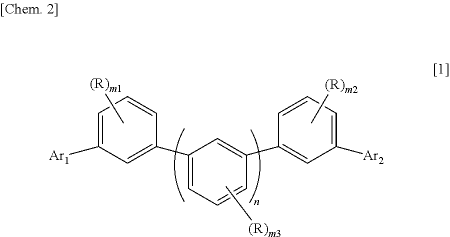

In the general formula [1], Ar₁ and Ar₂ are each independently selected from a substituted or unsubstituted tricyclic or polycyclic aryl group and a substituted or unsubstituted tricyclic or polycyclic heterocyclic group. Ar₁ and Ar₂ are represented by different skeletons. When Ar₁ and Ar₂ have a dibenzothiophene skeleton or a dibenzofuran skeleton, the organic compound has at least one substituent.

The substituent represented by R is selected from a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group. When a plurality of Rs are present, the Rs may be the same or different.

n denotes an integer in the range of 2 to 5, and m₁ to m₃ each denote an integer in the range of 0 to 4.

Further features of the present invention will become apparent from the following description of exemplary embodiments with reference to the attached drawings.

### BRIEF DESCRIPTION OF DRAWINGS

FIG. 1A is a schematic cross-sectional view of an example of a pixel of a display apparatus according to an embodiment of the present invention.

FIG. 1B is a schematic cross-sectional view of an example of a display apparatus including an organic light-emitting device according to an embodiment of the present invention.

FIG. 2 is a schematic view of an example of a display apparatus according to an embodiment of the present invention.

FIG. 3A is a schematic view of an example of an imaging apparatus according to an embodiment of the present invention.

FIG. 3B is a schematic view of an example of electronic equipment according to an embodiment of the present invention.

FIG. 4A is a schematic view of an example of a display apparatus according to an embodiment of the present invention.

FIG. 4B is a schematic view of an example of a foldable display apparatus.

FIG. 5A is a schematic view of an example of a lighting apparatus according to an embodiment of the present invention.

FIG. 5B is a schematic view of an example of an automobile with a vehicle lamp according to an embodiment of the present invention.

FIG. 6A is a schematic view of an example of a wearable device according to an embodiment of the present invention.

FIG. 6B is a schematic view of an example of a wearable device according to an embodiment of the present invention including an imaging apparatus.

FIG. 7A is a schematic view of an example of an image-forming apparatus according to an embodiment of the present invention.

FIG. 7B is a schematic view of an example of an exposure light source for an image-forming apparatus according to an embodiment of the present invention.

FIG. 7C is a schematic view of an example of an exposure light source for an image-forming apparatus according to an embodiment of the present invention.

FIG. 8 shows emission spectra of Exemplary Compound A4 and Comparative Compound 1-B obtained by a phosphorescence mode measurement.

### DESCRIPTION OF EMBODIMENTS

In the present description, the halogen atom is, for example, but not limited to, fluorine, chlorine, bromine, iodine, or the like.

The alkyl group may be an alkyl group with 1 or more and 20 or less carbon atoms. The alkyl group is, for example, but not limited to, a methyl group, an ethyl group, a n-propyl group, an isopropyl group, a n-butyl group, a tert-butyl group, a sec-butyl group, an octyl group, a cyclohexyl group, a 1-adamantyl group, a 2-adamantyl group, or the like.

The alkoxy group may be an alkoxy group with 1 or more and 10 or less carbon atoms. The alkoxy group is, for example, but not limited to, a methoxy group, an ethoxy group, a propoxy group, a 2-ethyl-octyloxy group, a benzyloxy group, or the like.

The amino group is, for example, but not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, an N-phenyl-N-(4-trifluoromethylphenyl)amino group, an N-piperidyl group, or the like.

The aryloxy group is, for example, but not limited to, a phenoxy group or the like.

The heteroaryloxy group is, for example, but not limited to, a thienyloxy group or the like.

The silyl group is, for example, but not limited to, a trimethylsilyl group, a triphenylsilyl group, or the like.

The aryl group may be an aryl group with 6 or more and 20 or less carbon atoms. The aryl group is, for example, but not limited to, a phenyl group, a naphthyl group, an indenyl group, a biphenyl group, a terphenyl group, a fluorenyl group, a phenanthryl group, a fluoranthenyl group, a triphenylenyl group, or the like.

The heterocyclic group may be a heterocyclic group with 3 or more and 20 or less carbon atoms. The heteroaryl group is, for example, but not limited to, a pyridyl group, an oxazolyl group, an oxadiazolyl group, a thiazolyl group, a thiadiazolyl group, a carbazolyl group, an acridinyl group, a phenanthrolyl group, a dibenzofuranyl group, a dibenzothiophenyl group, or the like.

An additional optional substituent of the alkyl group, the alkoxy group, the amino group, the aryloxy group, the silyl group, the aryl group, and the heterocyclic group may be, but is not limited to, a halogen atom, such as fluorine, chlorine, bromine, or iodine; an alkyl group, such as a methyl group, an ethyl group, a n-propyl group, an isopropyl group, a n-butyl group, or a t-butyl group; an alkoxy group, such as a methoxy group, an ethoxy group, or a propoxy group; an amino group, such as a dimethylamino group, a diethylamino group, a dibenzylamino group, a diphenylamino group, or a ditolylamino group; an aryloxy group, such as a phenoxy group; an aryl group, such as a phenyl group or a biphenyl group; a heterocyclic group, such as a pyridyl group or a pyrrolyl group; a cyano group, or the like.

### (1) Organic Compound

First, an organic compound according to the present invention is described below.

In the present description, the term “different skeletons” indicates that even identical skeletons are regarded as different if they are bound at different binding positions. More specifically, a 2-dibenzofuranyl group and a 3-dibenzofuranyl group have a dibenzofuran skeleton, but have different binding positions, and thus have different skeletons.

An organic compound according to the present invention is a compound represented by the general formula [1].

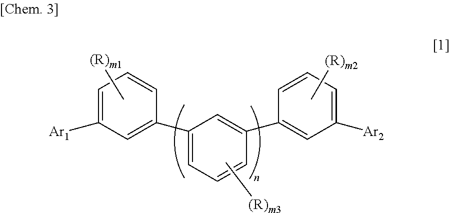

In the general formula [1], Ar₁ and Ar₂ are each independently selected from a substituted or unsubstituted tricyclic or polycyclic aryl group and a substituted or unsubstituted tricyclic or polycyclic heterocyclic group. Ar₁ and Ar₂ are represented by different skeletons. When Ar₁ and Ar₂ have a dibenzothiophene skeleton or a dibenzofuran skeleton, the organic compound has at least one substituent.

The substituent represented by R is selected from a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group. When a plurality of Rs are present, the Rs may be the same or different.

n denotes an integer in the range of 2 to 5, and m₁ to m₃ each denote an integer in the range of 0 to 4.

Furthermore, in Ar₁ and Ar₂ in the general formula [1], preferably, Ar₁ is a substituted or unsubstituted tricyclic or polycyclic aryl group, and Ar₂ is a substituted or unsubstituted tricyclic or polycyclic aryl group different from Ar₁, or a substituted or unsubstituted tricyclic or polycyclic heterocyclic group. This is because, as described later, an organic compound according to the present invention preferably has a molecular structure with low symmetry. Furthermore, Ar₁ and Ar₂ preferably have a large difference in electron density. This is because the charge symmetry is lowered. Thus, more preferably, one of Ar₁ and Ar₂ is a substituted or unsubstituted tricyclic or polycyclic aryl group, and the other is a substituted or unsubstituted tricyclic or polycyclic heterocyclic group. More specifically, the substituted or unsubstituted tricyclic or polycyclic aryl group is selected from a triphenylene skeleton, a fluorene skeleton, and a spirobifluorene skeleton. The substituted or unsubstituted tricyclic or polycyclic heterocyclic group is selected from a dibenzothiophene skeleton, a dibenzofuran skeleton, an azatriphenylene skeleton, an azadibenzothiophene skeleton, and an azadibenzofuran skeleton. The azadibenzothiophene skeleton refers to a dibenzothiophene skeleton with a nitrogen atom. The azadibenzofuran skeleton refers to a dibenzofuran skeleton with a nitrogen atom. More specifically, the substituted or unsubstituted tricyclic or polycyclic aryl group is the following substituent A group, and the substituted or unsubstituted tricyclic or polycyclic heterocyclic group is the following substituent B group.

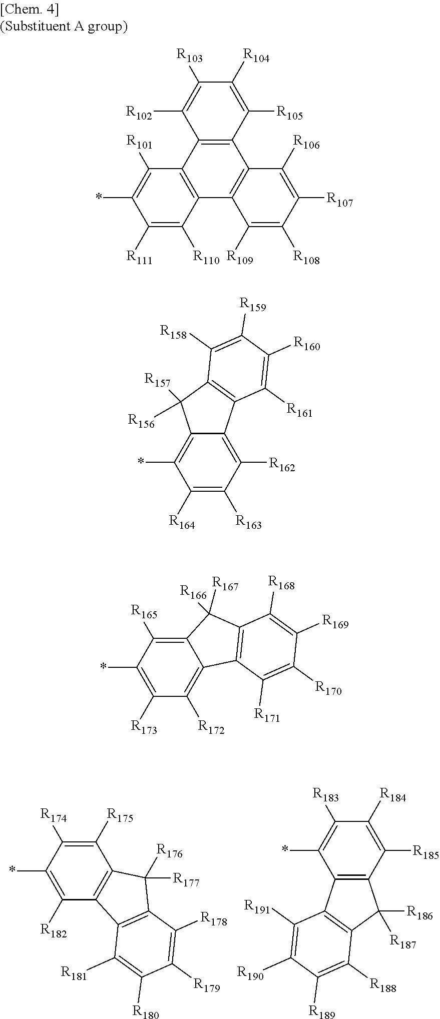

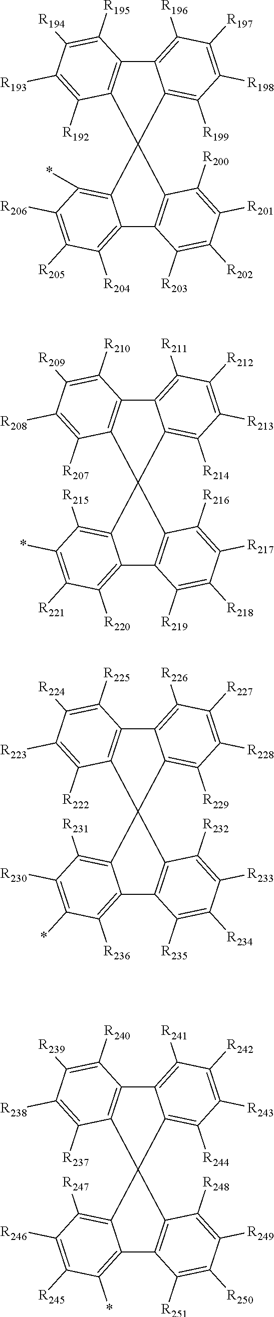

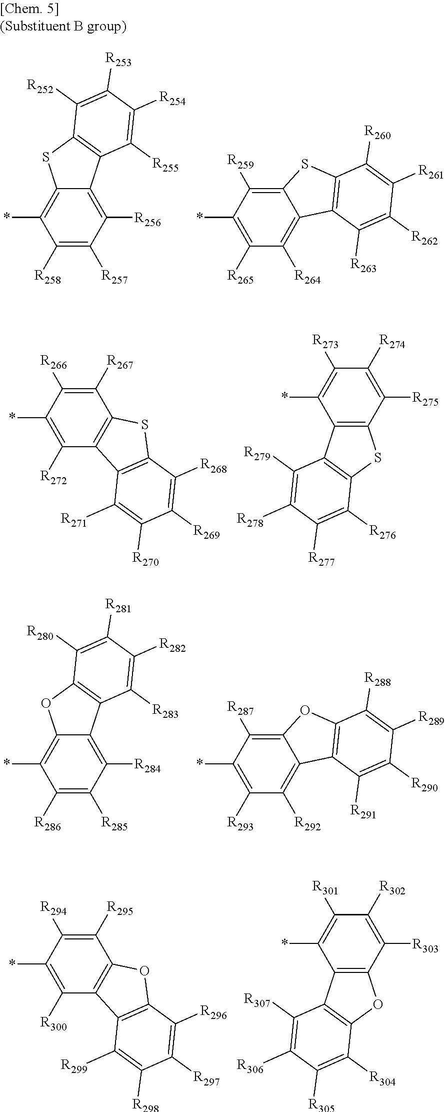

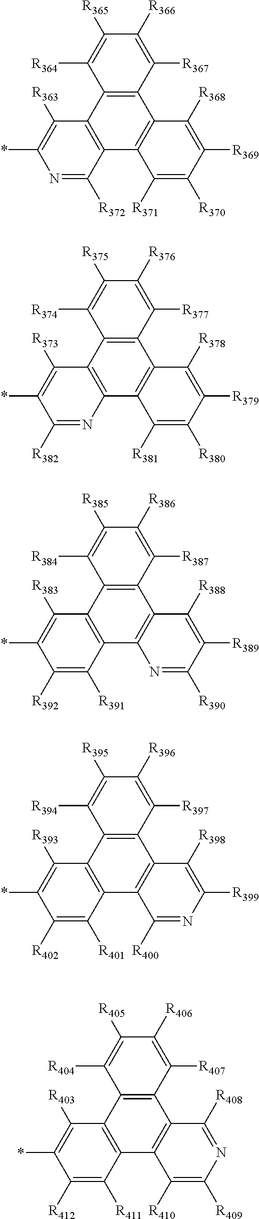

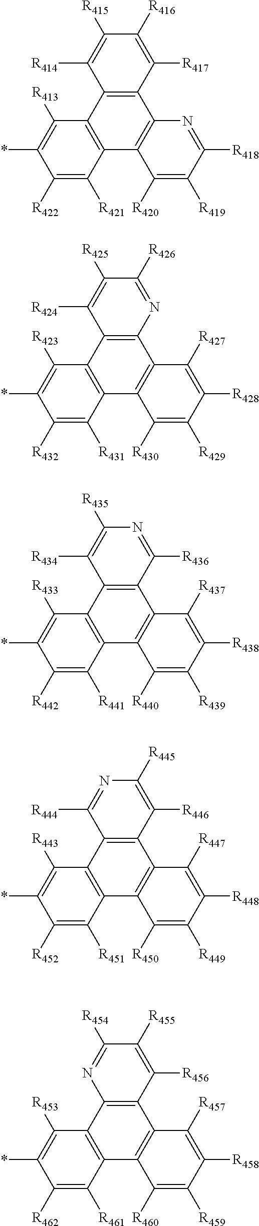

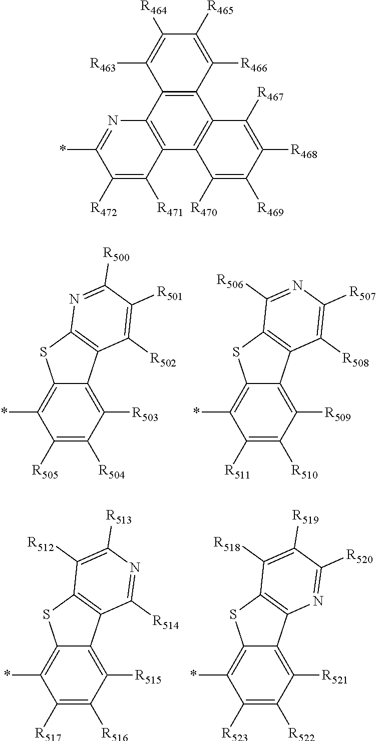

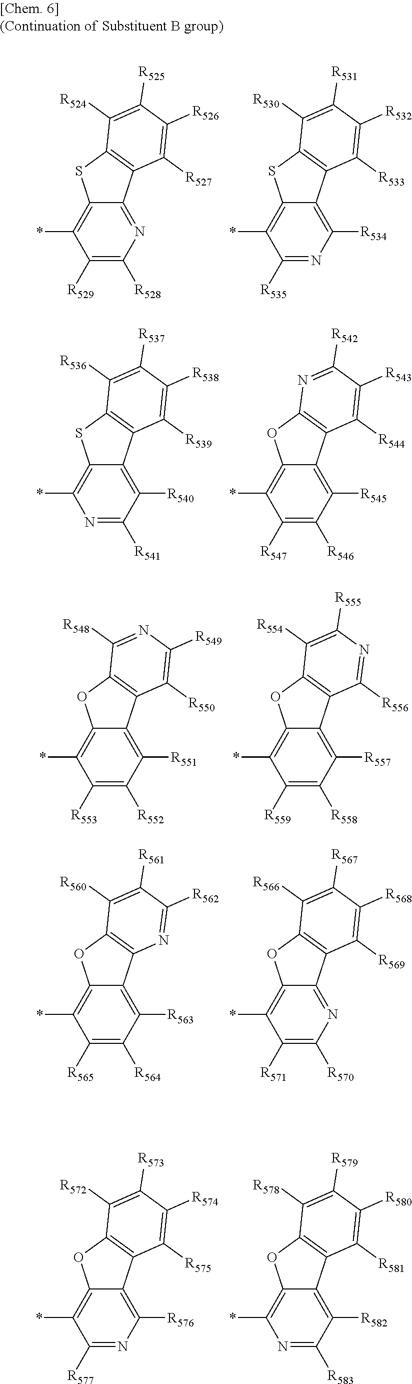

R₁₀₁ to R₅₈₃ in the substituent A group and the substituent B group are each independently selected from a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group. R₁₀₁ to R₅₈₃ are preferably selected from a hydrogen atom, a deuterium atom, an alkyl group with 1 to 4 carbon atoms, an aryl group with 6 to 18 carbon atoms, a heterocyclic group with 5 to 15 carbon atoms, a trimethylsilyl group, a triphenylsilyl group, and a cyano group, more preferably from a hydrogen atom, a phenyl group, and a tert-butyl group. * represents a binding position to a phenylene group.

The substituent A group is more preferably the following substituent C group. The substituent B group is more preferably the following substituent D group or substituent E group. This is because an oxygen atom, a sulfur atom, and a nitrogen atom in the substituent group D have a large number of lone pairs, and the substituent group have more electrons. This increases the difference in electron density between Ar₁ and Ar₂ and further reduces the molecular symmetry. When having a nitrogen atom, Ar₁ and Ar₂ have a low HOMO and a low LUMO (far from the vacuum level) and may lose carrier balance. A fused ring with a nitrogen atom is likely to have high nucleophilicity and may shorten the device life. Thus, Ar₁ and Ar₂ particularly preferably have no nitrogen atom. More specifically, one of Ar₁ and Ar₂ is a triphenylene skeleton, and the other is a dibenzothiophene skeleton or a dibenzofuran skeleton. More specifically, particularly preferably, one of Ar₁ and Ar₂ is the following substituent C group, and the other is the following substituent D group.

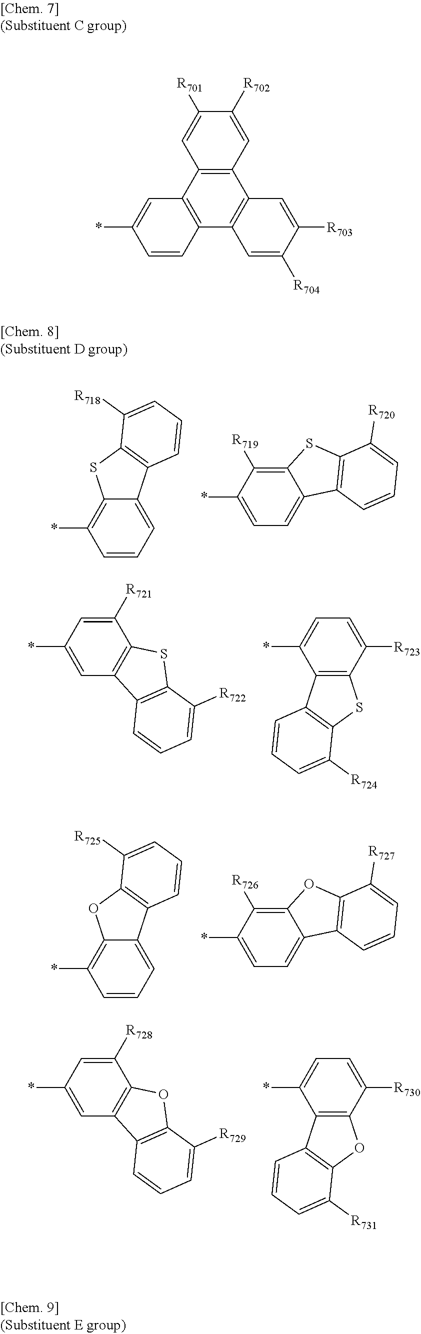

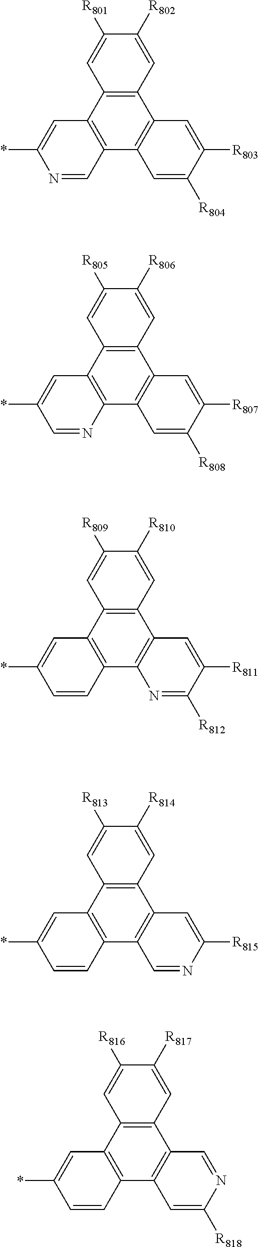

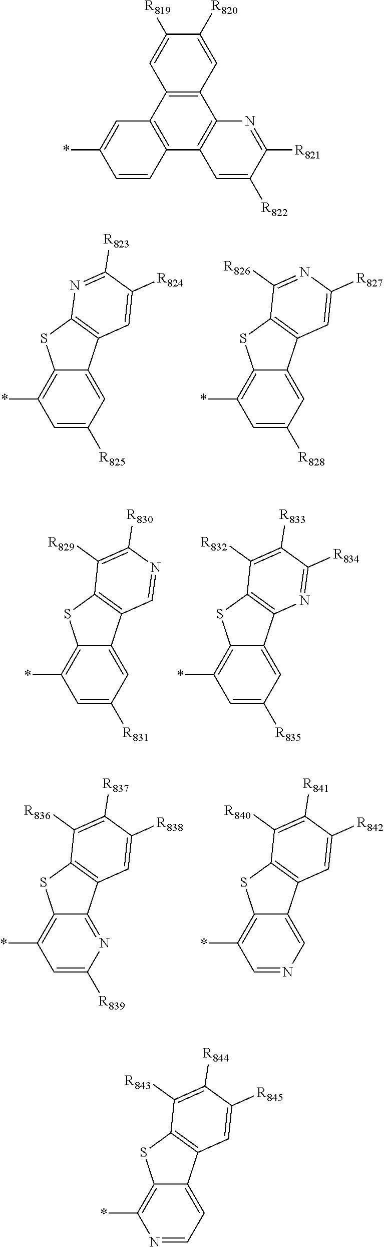

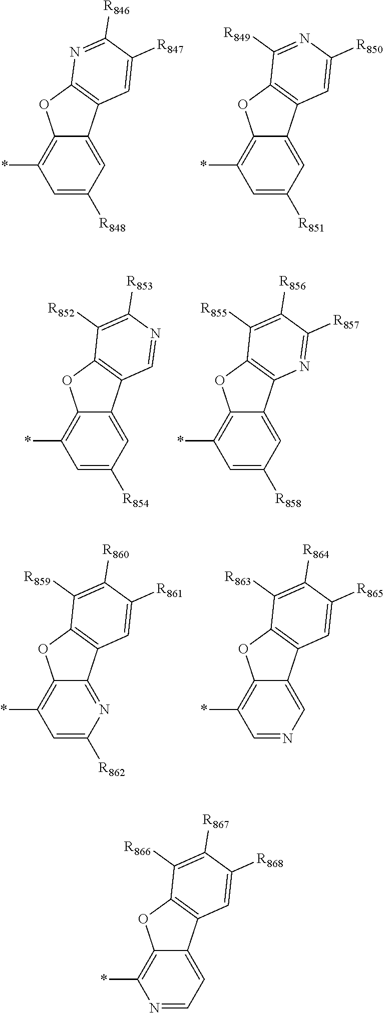

R₇₀₁ to R₈₆₈ in the substituent C group to the substituent E group are each independently selected from a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group. R₇₀₁ to R₈₆₈ are preferably selected from a hydrogen atom, a deuterium atom, an alkyl group with 1 to 4 carbon atoms, an aryl group with 6 to 18 carbon atoms, a heterocyclic group with 5 to 15 carbon atoms, a trimethylsilyl group, a triphenylsilyl group, and a cyano group, more preferably from a hydrogen atom, a phenyl group, and a tert-butyl group. * represents a binding position to a phenylene group.

The organic compound represented by the general formula (1) has the following characteristics.

(1-1) Having a phenylene chain bonded to four or more benzenes increases the glass transition temperature (Tg) and improve the thermal stability of a film.

(1-2) Benzene constituting the phenylene chain and bonded at a meta (m) position increases the triplet (T1) energy.

(1-3) Different structures of Ar₁ and Ar₂ bonded to the phenylene chain reduce the molecular symmetry and suppress the aggregation of molecules in a film.

(1-4) The structures of (1-1) to (1-3) improve sublimability.

These characteristics are described below.

(1-1) Having a phenylene chain bonded to four or more benzenes increases the glass transition temperature (Tg) and improve the thermal stability of a film.

In inventing an organic compound according to the present invention, the present inventors focused on the structure of the phenylene chain.

More specifically, an organic compound according to the present invention has a phenylene chain bonded to four or more benzenes. This increases the molecular weight of the compound itself and Tg. Consequently, an organic compound according to the present invention provides a film with high thermal stability.

Table 1 shows the Tg of Exemplary Compounds A3 and A4 according to an embodiment of the present invention and the Tg of Comparative Compound 1-A. The Tg of each compound was evaluated by differential scanning calorimetry (DSC). Approximately 2 mg of a sample in an aluminum pan was rapidly cooled from a high temperature exceeding the melting point. After the sample was brought into an amorphous state, Tg was measured by increasing the temperature at a heating rate of 10° C./min. The measuring apparatus was DSC 204 F1 manufactured by NETZSCH.

TABLE 1

| | | Glass |
|---|---|---|
| | | transition |
| | | temperature |
| | Structure | (° C.) |
| Exemplary Compound A3 | 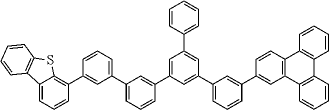 [SMILES: c1ccc(-c2cc(-c3cccc(-c4cccc(-c5cccc6c5sc5ccccc56)c4)c3)cc(-c3cccc(-c4ccc5c6ccccc6c6ccccc6c5c4)c3)c2)cc1] | 136 |
| Exemplary Compound A4 | 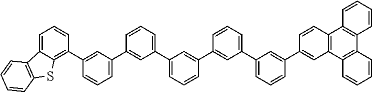 [SMILES: c1cc(-c2cccc(-c3cccc(-c4ccc5c6ccccc6c6ccccc6c5c4)c3)c2)cc(-c2cccc(-c3cccc(-c4cccc5c4sc4ccccc45)c3)c2)c1] | 149 |
| Comparative Compound 1-A | 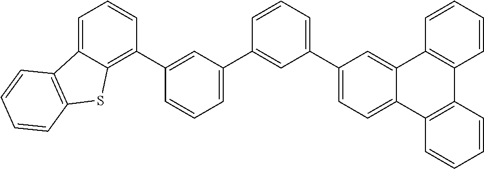 [SMILES: c1cc(-c2cccc(-c3cccc4c3sc3ccccc34)c2)cc(-c2ccc3c4ccccc4c4ccccc4c3c2)c1] | 108 |

In Table 1, Comparative Compound 1-A had a Tg of 108° C. and had a low Tg. Thus, Comparative Compound 1-A provides a film with unfavorable thermal stability.

On the other hand, Exemplary Compounds A3 and A4 have a Tg of 136° C. and 149° C., respectively, and have a high Tg. Thus, Exemplary Compounds A3 and A4 provide a film with high thermal stability. Thus, an organic compound according to the present invention can be used for an organic light-emitting device to form a film with high thermal stability and to provide an organic light-emitting device with long device life.

(1-2) Benzene constituting the phenylene chain and bonded at a meta (m) position increases the triplet (T1) energy.

In inventing an organic compound according to the present invention, the present inventors focused on the structure of the phenylene chain.

More specifically, an organic compound according to the present invention has high T1 energy due to the benzene constituting the phenylene chain and bonded at a m-position.

Table 2 shows the T1 energy of Exemplary Compound A4 according to an embodiment of the present invention and Comparative Compound 1-B. The T1 energy was measured at 77 K with F-4500 manufactured by Hitachi, Ltd. by photoluminescence (PL) measurement of a diluted toluene solution and a deposited film at an excitation wavelength of 300 nm. It was then calculated from a rising emission end of a light emission spectrum on the short-wavelength side obtained by phosphorescence mode measurement in F-4500. A deposited film sample deposited on a quartz substrate in a vacuum of 5×10⁻⁴ Pa or less was used for the measurement.

TABLE 2

| | | | Deposited | |
|---|---|---|---|---|
| | | Solution | film T1 | ΔT1 |
| | Structure | T1 (eV) | (eV) | (eV) |
| Exemplary Compound A4 | 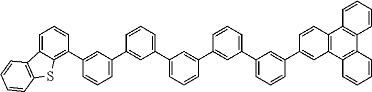 [SMILES: c1cc(-c2cccc(-c3cccc(-c4ccc5c6ccccc6c6ccccc6c5c4)c3)c2)cc(-c2cccc(-c3cccc(-c4cccc5c4sc4ccccc45)c3)c2)c1] | 2.75 | 2.57 | 0.18 |
| Comparative Compound 1-B | 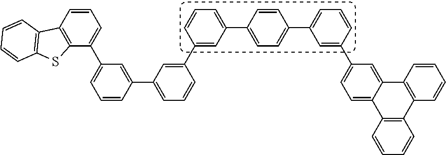 [SMILES: c1cc(-c2ccc(-c3cccc(-c4ccc5c6ccccc6c6ccccc6c5c4)c3)cc2)cc(-c2cccc(-c3cccc(-c4cccc5c4sc4ccccc45)c3)c2)c1] | 2.75 | 2.50 | 0.25 |

The term “solution T1” refers to the T1 energy of each compound dissolved in toluene, and the term “deposited film T1” refers to the T1 energy of a film formed by a vapor deposition method. The term “ΔT1” refers to the difference between the solution T1 and the deposited film T1.

Table 2 shows that Exemplary Compound A4 has a higher deposited film T1 than Comparative Compound 1-B.

The effects of high T1 energy are described below.

Phosphorescent devices are organic light-emitting devices that use the T1 energy for light emission. A host material or a peripheral material of a light-emitting layer of an organic light-emitting device needs to have T1 energy sufficiently higher than that of a phosphorescent material that emits phosphorescence.

For example, a host material of a light-emitting layer that does not have a sufficiently higher T1 energy than a phosphorescent material cannot transfer sufficient energy to the phosphorescent material, resulting in lower light emission efficiency.

When a host material of a light-emitting layer does not have a sufficiently higher T1 energy than a phosphorescent material, reverse energy transfer from the phosphorescent material to the host material is likely to occur. Thus, the host material has an unstable triplet state for a long time and may be degraded. This undesirably shortens the device life. Thus, the host material preferably has a sufficiently high T1 energy. This can promote energy transfer from the host material to the phosphorescent material and improve the device life.

More specifically, in an organic light-emitting device that emits green phosphorescence, the T1 energy of a host material is preferably higher than 2.43 eV (510 nm in wavelength) by 0.1 eV or more. In an organic light-emitting device that emits red phosphorescence, the T1 energy of a host material is preferably higher than 2.07 eV (600 nm in wavelength) by 0.1 eV or more.

In Table 2, Exemplary Compound A4 has a deposited film T1 of 2.57 eV and is therefore more preferred than Comparative Compound 1-B as a host material of an organic light-emitting device that emits green and red phosphorescence.

Table 2 shows that Comparative Compound 1-B has a larger ΔT1 than Exemplary Compound A4. That is, the T1 energy of Comparative Compound 1-B is likely to decrease during film formation. Comparative Compound 1-B has a structure in which benzene constituting a phenylene chain is bonded at the para (p) position (the square dotted line portion in Table 2), and molecules are therefore likely to aggregate in a film. This probably results in lower T1 energy. On the other hand, in Exemplary Compound A4, all benzenes constituting a phenylene chain are bonded at the m-position, and molecules are therefore less likely to aggregate in a film. This probably results in higher T1 energy.

Effects of molecules less likely to aggregate in a film are described below.

Molecules likely to aggregate are unfavorable from the perspective of carrier transport ability or light emission efficiency because a grain boundary, a trap level, or a quencher due to fine crystallization is easily formed. On the other hand, molecules less likely to aggregate can maintain good carrier transport properties and efficient emission properties because a grain boundary, a trap level, or a quencher due to fine crystallization is less likely to be formed. Consequently, an organic light-emitting device with long device life and high light emission efficiency can be provided.

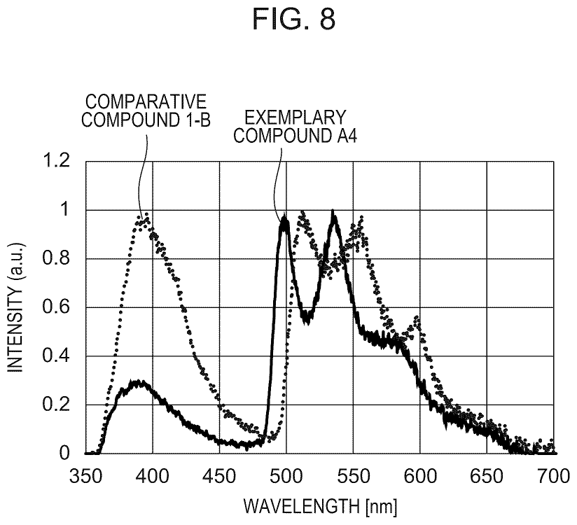

FIG. 8 shows that Comparative Compound 1-B had a higher emission intensity derived from singlet (S1) energy near a wavelength of 400 nm in phosphorescence mode measurement than Exemplary Compound A4. Thus, Comparative Compound 1-B has a longer fluorescence lifetime (excitation lifetime) in the S1 state than Exemplary Compound A4. Comparative Compound 1-B is considered to have a long fluorescence lifetime (excitation lifetime) because molecules are likely to overlap in a film and the intermolecular interaction is large. As shown in the following formula, the rate constant of Forster energy transfer is inversely proportional to the fluorescence lifetime (excitation lifetime) of the host material. Consequently, the use of Comparative Compound 1-B in an organic light-emitting device is unfavorable due to lower light emission efficiency.

$$[ Math . 1 ]$$

$${k}_{FRET} = \frac{9000 {c}^{4} {K}^{2} Φ ( ln 10 )}{128 {π}^{5} {n}^{4} N τ {R}^{6}} \int {f}_{H} ( λ ) {ε}_{D} ( λ ) {λ}^{4} d λ$$

c: speed of light
K²: dipole orientation factor
Φ: emission quantum yield
n: refractive index of medium
N: Avogadro's number
τ: fluorescence lifetime of host
R: intermolecular distance between host and guest
f_{H}(λ): normalized emission spectrum of host
ϵ_{D}(λ): molar absorption coefficient of guest

Furthermore, a host material likely to aggregate may cause aggregation of a guest material that is a light-emitting material. Aggregation of the guest material causes concentration quenching, and the host material likely to aggregate is therefore unfavorable. On the other hand, Exemplary Compound A4 is preferred from the perspective of emission properties of an organic light-emitting device because the host material is less likely to aggregate and a light-emitting layer in which the host material and the guest material are dispersed can be formed.

(1-3) Different structures of Ar₁ and Ar₂ bonded to the phenylene chain reduce the molecular symmetry and suppress the aggregation of molecules in a film.

In inventing an organic compound according to the present invention, the present inventors focused on the structure of Ar₁ and Ar₂.

In the general formula [1], Ar₁ and Ar₂ are different skeletons. Thus, in an organic compound according to the present invention, the symmetry of the molecular structure of the compound is reduced, and the molecules are less likely to aggregate in a film.

Effects due to the low symmetry of the molecular structure of the compound are described below.

Lower symmetry can suppress molecular packing of overlapping molecules and suppress the aggregation of the molecules. Thus, the molecules are less likely to crystallize, and the amorphous state is easily maintained. It is preferable that the amorphous state be easily maintained in a case of being used in an organic light-emitting device. This is because maintaining the amorphous state can reduce the formation of a grain boundary, a trap level, or a quencher associated with fine crystallization even during the operation of the device and can maintain good carrier transport properties and efficient emission properties. Consequently, an organic light-emitting device with long device life and high light emission efficiency can be provided.

The compound having a molecular structure with low symmetry can suppress the overlap of molecules and reduce intermolecular interaction. Thus, high T1 energy can be maintained even during film formation.

Table 3 shows the results of comparing the symmetry of Exemplary Compound A4, which is an embodiment of the present invention, with Comparative Compound 1-C and Comparative Compound 1-D.

TABLE 3

| | | Glass | | | |
|---|---|---|---|---|---|
| | | transition | | Deposited | |
| | | temperature | Solution | film T1 | |
| | Structure | (° C.) | T1 (eV) | (eV) | ΔT1 |
| Exemplary Compound A4 | 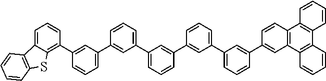 [SMILES: c1cc(-c2cccc(-c3cccc(-c4ccc5c6ccccc6c6ccccc6c5c4)c3)c2)cc(-c2cccc(-c3cccc(-c4cccc5c4sc4ccccc45)c3)c2)c1] | 149 | 2.75 | 2.57 | 0.18 |
| Comparative Compound 1-C | 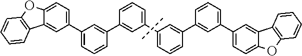 | 95 | 2.95 | 2.65 | 0.30 |
| Comparative Compound 1-D | 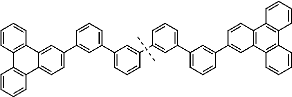 | 139 | 2.74 | 2.52 | 0.22 |

Comparative Compound 1-C and Comparative Compound 1-D, which have the same structure at Ar₁ and Ar₂ when the molecular structure is viewed on a plane, have a highly symmetric structure. More specifically, they have a 2-fold rotation axis on the dotted line in Table 3. On the other hand, Exemplary Compound A4 has Ar₁ and Ar₂ with different structures and does not have a rotation axis on the molecular plane. Thus, Exemplary Compound A4 has lower symmetry than Comparative Compound 1-C and Comparative Compound 1-D.

Table 3 shows that Comparative Compound 1-C had a Tg of 95° C., whereas Exemplary Compound A4 had a Tg of 149° C. Thus, as described above, Exemplary Compound A4 is an organic compound with higher thermal stability than Comparative Compound 1-C.

Comparative Compound 1-C and Comparative Compound 1-D having a molecular structure with high symmetry had a ΔT1 of 0.30 eV and 0.22 eV, respectively. On the other hand, Exemplary Compound A4 having a molecular structure with low symmetry had ΔT1 of 0.18 eV, which was smaller than ΔT1 of Comparative Compound 1-C and Comparative Compound 1-D. Thus, the T1 energy of Exemplary Compound A4 is less likely to decrease when a deposited film is formed. Thus, an organic compound according to the present invention is preferred for an organic light-emitting device due to its ease in achieving high T1 energy.

In Comparative Compound 1-C and Comparative Compound 1-D having a molecular structure with high symmetry, molecules are likely to aggregate due to large ΔT1. As described above, a compound in which a host material is likely to aggregate is unfavorable because a trap level or a quencher is easily formed.

Regarding the structures of Ar₁ and Ar₂ in an organic compound according to the present invention, preferably, one of Ar₁ and Ar₂ is a substituted or unsubstituted aryl group, and the other is a substituted or unsubstituted aryl group or a substituted or unsubstituted heterocyclic group. More preferably, one of Ar₁ and Ar₂ is a substituted or unsubstituted aryl group, and the other is a substituted or unsubstituted heterocyclic group. This is because the difference in electron density is increased, and the molecular symmetry is further reduced. This can further suppress the overlap of molecules. More specifically, preferably, one of Ar₁ and Ar₂ is the substituent C group, and the other is the substituent D group or the substituent E group. As described above, Ar₁ and Ar₂ particularly preferably have no nitrogen atom. Thus, particularly preferably, one of Ar₁ and Ar₂ is the substituent C group, and the other is the substituent D group.

(1-4) The structures of (1-1) to (1-3) improve sublimability.

Having the structures (1-1) to (1-3), an organic compound according to the present invention has high sublimability. More specifically, the compound has high sublimability due to a phenylene chain having four or more benzenes bonded at the m-position and due to Ar₁ and Ar₂ with different structures.

Table 4 shows the results of comparing the sublimability of Exemplary Compound A4, which is an embodiment of the present invention, with Comparative Compound 1-B and Comparative Compound 1-D.

For the evaluation of sublimability, the temperature difference (ΔT) between the sublimation temperature and the decomposition temperature is compared. A larger ΔT indicates higher sublimability. The decomposition temperature is a temperature at which the weight loss reaches 5% in thermogravimetry/differential thermal analysis (TG/DTA). The sublimation temperature is a temperature at which a sufficient sublimation rate is achieved while the temperature is slowly increased in a vacuum of 1×10⁻¹ Pa in an Ar₁ flow to perform sublimation purification. A sufficient sublimation rate may be 0.01 g/min.

TABLE 4

| | | | ΔT = Decomposition |
|---|---|---|---|
| | | Molecular | temperature − sublimation |
| | Structure | weight | temperature |
| Exemplary Compound A4 | 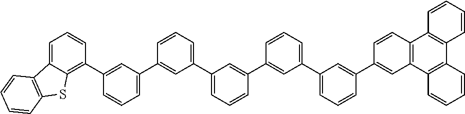 [SMILES: c1cc(-c2cccc(-c3cccc(-c4ccc5c6ccccc6c6ccccc6c5c4)c3)c2)cc(-c2cccc(-c3cccc(-c4cccc5c4sc4ccccc45)c3)c2)c1] | 791 | 70 |
| Comparative Compound 1-B | 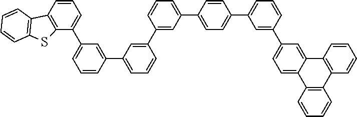 [SMILES: c1cc(-c2ccc(-c3cccc(-c4ccc5c6ccccc6c6ccccc6c5c4)c3)cc2)cc(-c2cccc(-c3cccc(-c4cccc5c4sc4ccccc45)c3)c2)c1] | 791 | 60 |
| Comparative Compound 1-D | 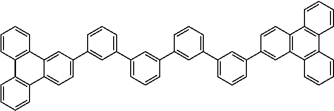 [SMILES: c1cc(-c2cccc(-c3cccc(-c4ccc5c6ccccc6c6ccccc6c5c4)c3)c2)cc(-c2cccc(-c3ccc4c5ccccc5c5ccccc5c4c3)c2)c1] | 759 | 50 |

Table 4 shows that Exemplary Compound A4 has a larger ΔT than Comparative Compound 1-B. Furthermore, even having a higher molecular weight, Exemplary Compound A4 has a larger ΔT than Comparative Compound 1-D. Thus, it can be said that Exemplary Compound A4 has higher sublimability than Comparative Compound 1-B and Comparative Compound 1-D.

This can be explained as follows.

As described above, since Exemplary Compound A4 has the phenylene chain having four or more benzenes bonded at the m-position, the conjugation length is less likely to extend, and the overlap of molecules can be suppressed. Furthermore, Ar₁ and Ar₂ with different structures reduce the symmetry of the molecular structure and can suppress the overlap of molecules. Thus, the compound has high sublimability. On the other hand, since Comparative Compound 1-B has benzene constituting the phenylene chain and bonded at the p-position, the conjugation length extends, and molecules are likely to overlap. In Comparative Compound 1-D, since Ar₁ and Ar₂ have the same structure, the molecular symmetry is high, and molecules are likely to overlap. Thus, the compound has unfavorable sublimability.

A compound with high sublimability is less likely to decompose during sublimation purification. This indicates high deposition stability in the production of an organic light-emitting device. Thus, a high-purity deposited film can be produced, and an organic light-emitting device with long device life can be provided.

An organic compound according to the present invention preferably further has the following characteristics because the organic compound can be suitably used for an organic light-emitting device. Two or more of the following conditions may be simultaneously satisfied.

(1-5) In the general formula [1], Ar₁ and Ar₂ with no SP3 carbon result in high binding stability.

(1-6) In the general formula [1], the substituent represented by R and bonded at the m-position of the benzene constituting the phenylene chain results in high binding stability.

(1-7) In the general formula [1], when n is 3 or 4, molecules are less likely to aggregate in a film.

These characteristics are described below.

(1-5) In the general formula [1], Ar₁ and Ar₂ with no SP3 carbon result in high binding stability.

In an organic compound according to the present invention, Ar₁ and Ar₂ preferably have no SP3 carbon. This is because a carbon-carbon bond with SP3 carbon has low binding energy and is easily cleaved during the operation of the organic light-emitting device. Ar₁ and Ar₂ with no SP3 carbon can suppress bond cleavage. Thus, an organic compound according to the present invention is preferably used for an organic light-emitting device to provide the organic light-emitting device with long device life.

When Ar₁ and Ar₂ have a substituent, the substituent preferably has no SP3 carbon. Particularly preferably, Ar₁ and Ar₂ have no substituent.

(1-6) In the general formula [1], the substituent represented by R and bonded at the m-position of the benzene constituting the phenylene chain results in high binding stability.

In an organic compound according to the present invention, the substituent represented by R is preferably bonded at a substitution position where the substituent has small interference with the phenylene chain due to steric hindrance. More specifically, the substituent represented by R is preferably bonded at the m-position of benzene constituting the phenylene chain. This is because interference due to steric hindrance between the substituent represented by R and the phenylene chain increases the bond length between the substituent and the phenylene chain. This reduces binding stability and accelerates bond cleavage.

Table 5 shows the results of comparison of the bond length between the phenylene chain and the substituent of Exemplary Compound A5 and Exemplary Compound A22 according to embodiments of the present invention. In Table 5, “a” indicates a bonding site between the phenylene chain and the substituent in each of the compared exemplary compounds.

TABLE 5

| | | Bond |
|---|---|---|
| | | length |
| | Structure | (Å) |
| Exemplary Compound A5 | 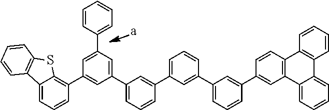 [SMILES: c1ccc(-c2cc(-c3cccc(-c4cccc(-c5cccc(-c6ccc7c8ccccc8c8ccccc8c7c6)c5)c4)c3)cc(-c3cccc4c3sc3ccccc34)c2)cc1] | 1.486 |
| Exemplary Compound A22 | 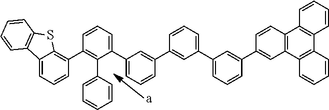 [SMILES: c1ccc(-c2c(-c3cccc(-c4cccc(-c5cccc(-c6ccc7c8ccccc8c8ccccc8c7c6)c5)c4)c3)cccc2-c2cccc3c2sc2ccccc23)cc1] | 1.497 |

Table 5 shows that the bond length between the phenylene chain and the substituent in Exemplary Compound A5 was 1.486 angstroms, whereas the bond length between the phenylene chain and the substituent in Exemplary Compound A22 was 1.497 angstroms. This is because Exemplary Compound A22 is more susceptible to the steric hindrance of hydrogen atoms than Exemplary Compound A5.

Thus, the substituent and the phenylene chain are preferably bonded at a substitution position with small interference due to steric hindrance. More specifically, the substituent is preferably bonded at the m-position of benzene constituting the phenylene chain. Furthermore, the substituent is preferably a substituent with small interference due to steric hindrance. More specifically, an aryl group with 6 to 18 carbon atoms and a heterocyclic group with 5 to 9 carbon atoms are preferred. In particular, from the perspective of binding stability, a phenyl group or a pyridyl group is more preferred.

In the general formula [1], when Ar₁ and Ar₂ are a dibenzofuran skeleton or a dibenzothiophene skeleton, an organic compound according to the present invention has at least one substituent. This further improves Tg and is preferred. In particular, a substituent at the m-position of the benzene constituting the phenylene chain is more preferred because it is less likely to extend the conjugation length and easily maintains high T1. The substituent is preferably a phenyl group. This is because a phenyl group as a substituent can suppress the aggregation of molecules and improve the planarity of the molecules and Tg.

Furthermore, an organic compound according to the present invention preferably has no substituent on the phenylene chain because interference due to steric hindrance between the substituent and the phenylene chain is less likely to occur. Table 6 shows the results of comparing the bond lengths of Exemplary Compound A2 and Exemplary Compound A5 according to embodiments of the present invention. In Table 6, “b” indicates a bond with the maximum bond length of the compound.

TABLE 6

| | Structure | Maximum bond length (Å) |
|---|---|---|
| Exemplary Compound A2 | 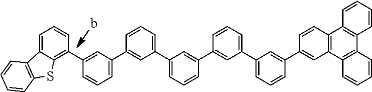 [SMILES: c1cc(-c2cccc(-c3cccc(-c4ccc5c6ccccc6c6ccccc6c5c4)c3)c2)cc(-c2cccc(-c3cccc(-c4cccc5c4sc4ccccc45)c3)c2)c1] | 1.488 |
| Exemplary Compound A5 | 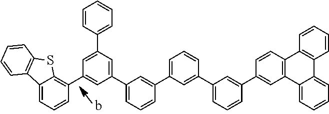 [SMILES: c1ccc(-c2cc(-c3cccc(-c4cccc(-c5cccc(-c6ccc7c8ccccc8c8ccccc8c7c6)c5)c4)c3)cc(-c3cccc4c3sc3ccccc34)c2)cc1] | 1.489 |

Table 6 shows that Exemplary Compound A2 had a maximum bond length of 1.488 angstroms, whereas Exemplary Compound A5 had a maximum bond length of 1.489 angstroms. Exemplary Compound A2 has no substituent on the phenylene chain and is therefore less susceptible to interference due to steric hindrance between the substituent and the phenylene chain. This further reduces the maximum bond length, and the compound has particularly high binding stability. Thus, an organic compound according to the present invention most preferably has no substituent on the phenylene chain. In other words, in the general formula [1], m is particularly preferably 0.

(1-7) In the general formula [1], when n is 3 or 4, molecules are less likely to aggregate in a film.

In the general formula [1], n is preferably 3 or 4 because molecules of an organic compound according to the present invention are less likely to aggregate in a film. This is because, as n increases, the molecular weight of the compound tends to increase, and Tg also tends to increase. Furthermore, as the phenylene chain extends, the number of conformations of the molecular structure increases, and the molecules are less likely to aggregate. In particular, n is preferably 3 or 4 because high sublimability can be maintained.

Specific examples of an organic compound according to the present invention are described below. However, the present invention is not limited thereto.

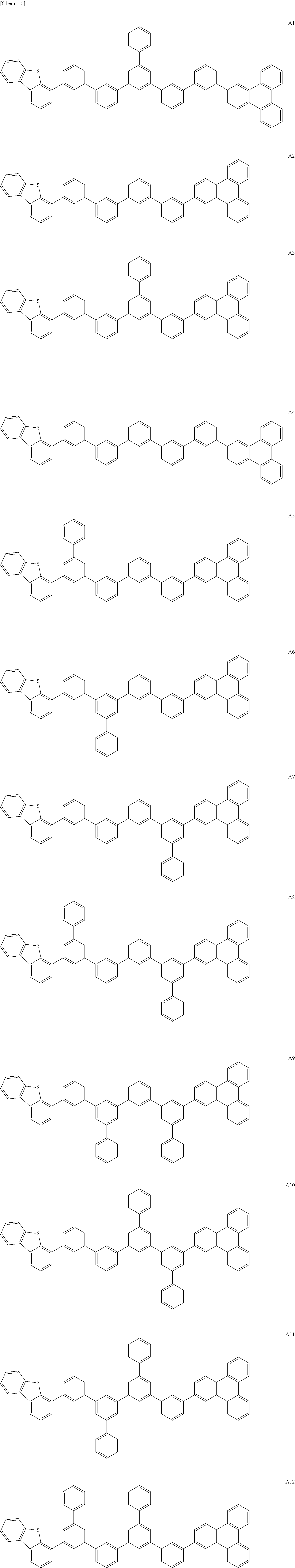

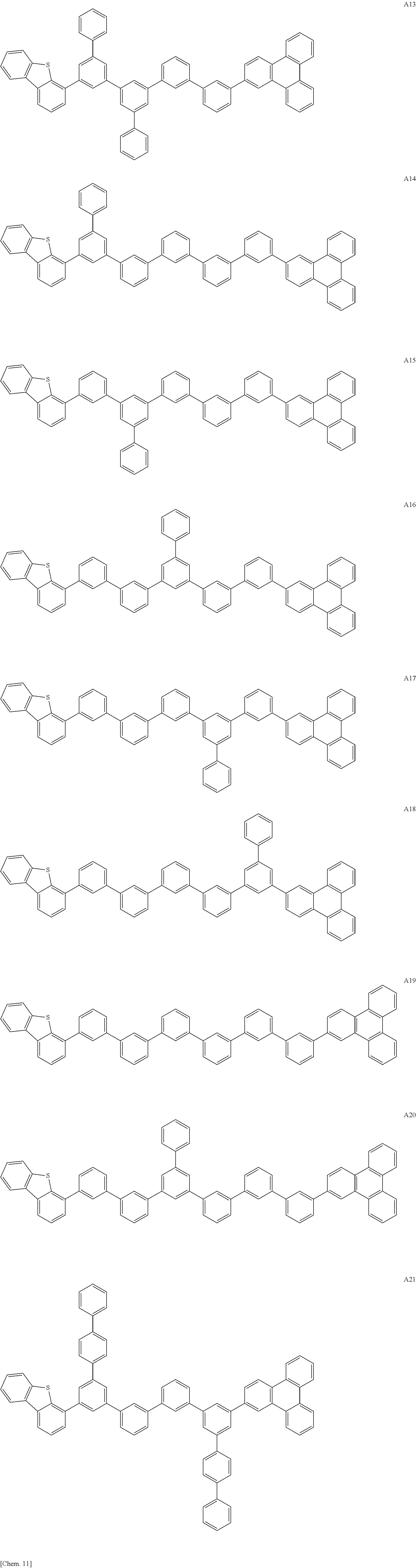

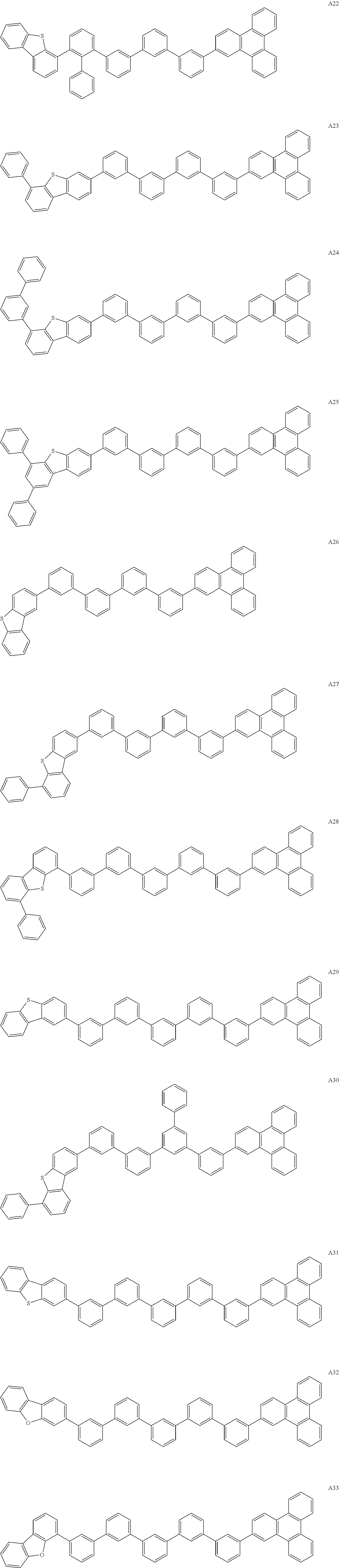

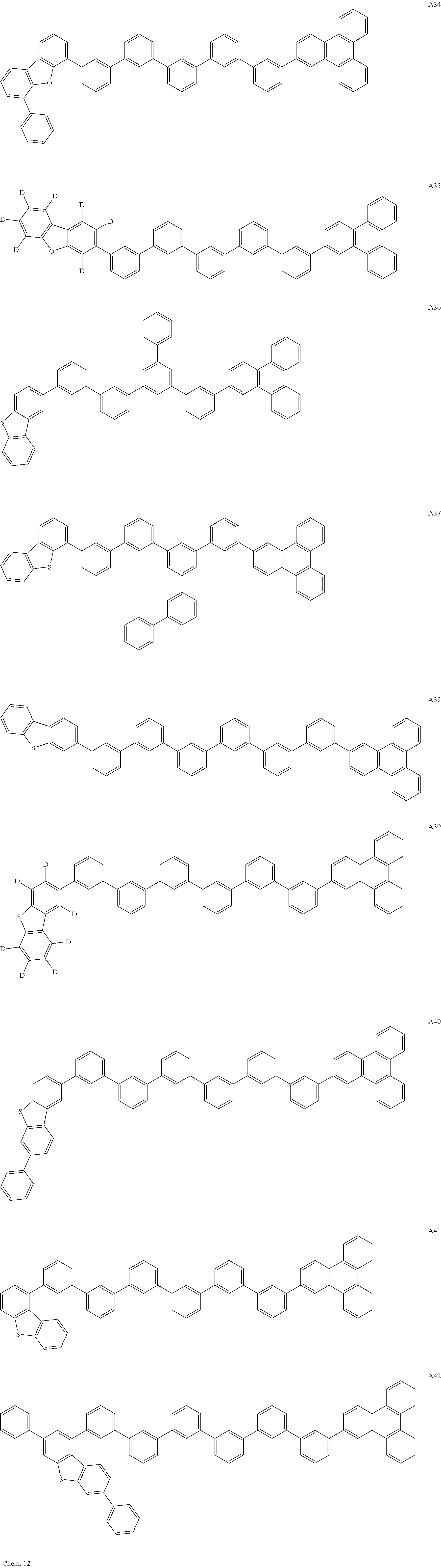

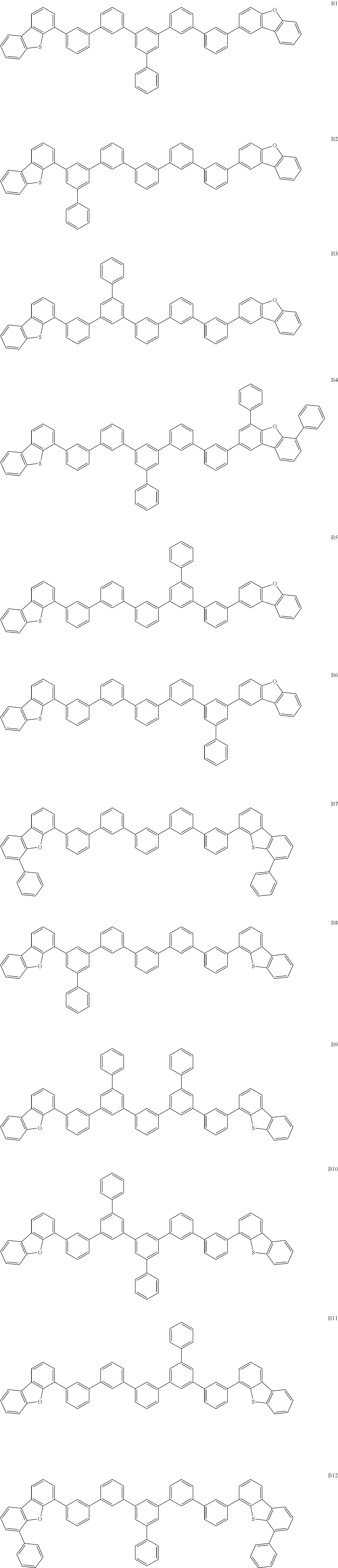

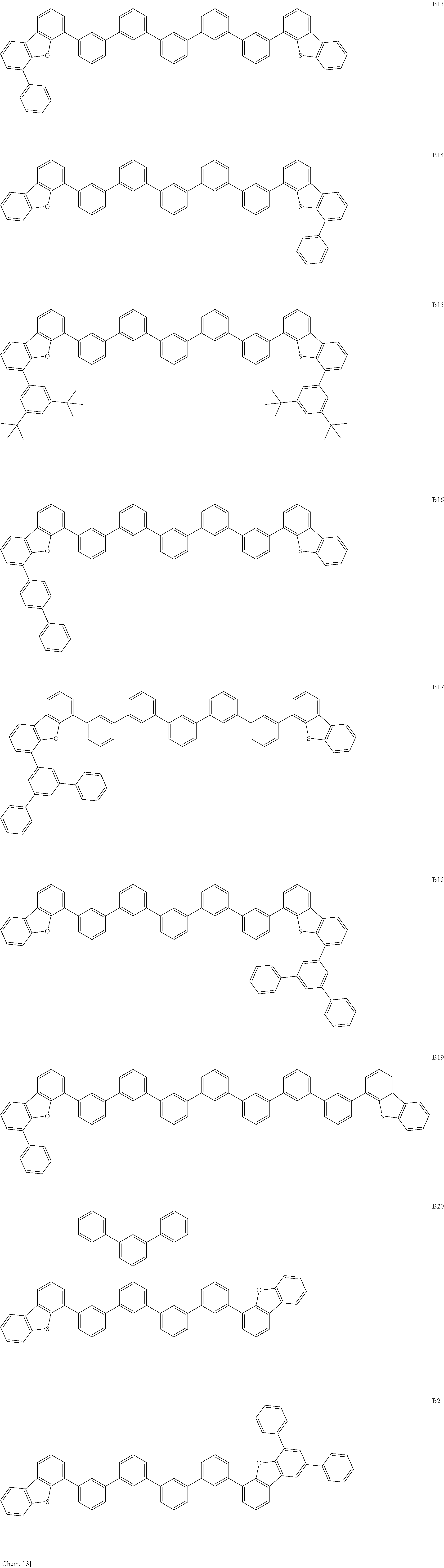

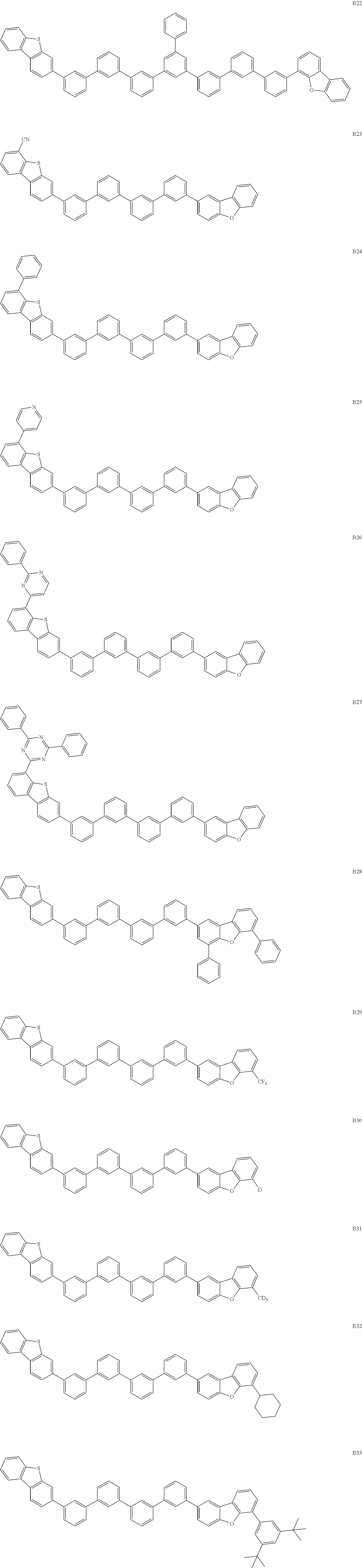

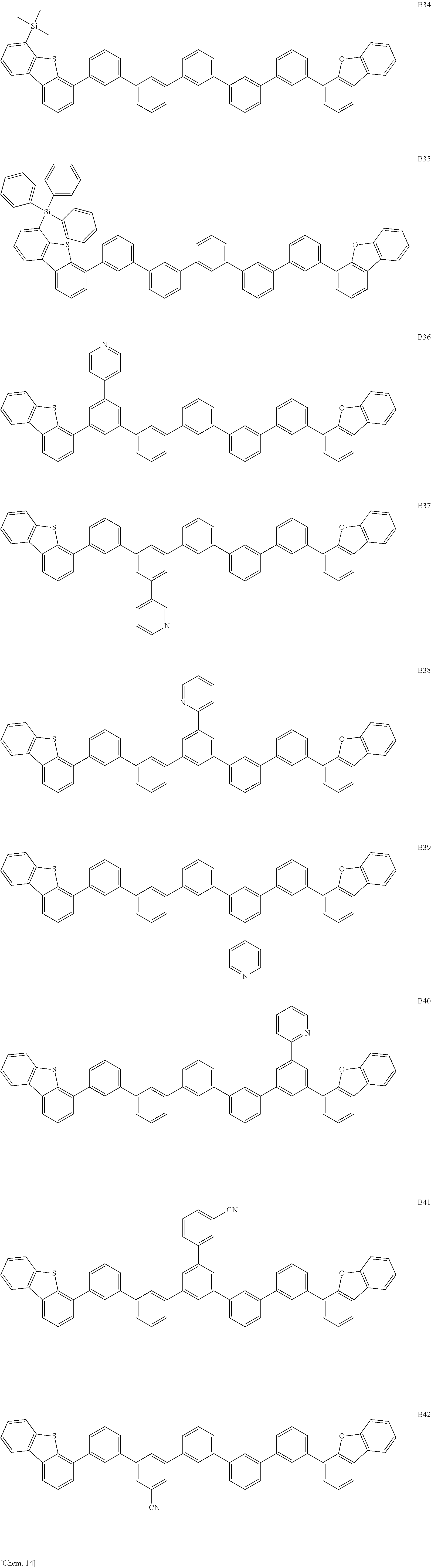

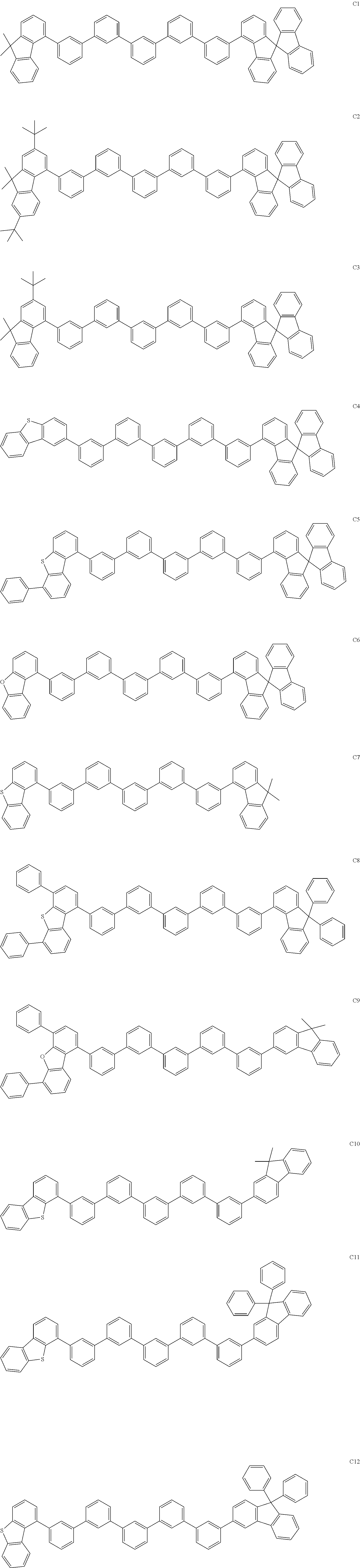

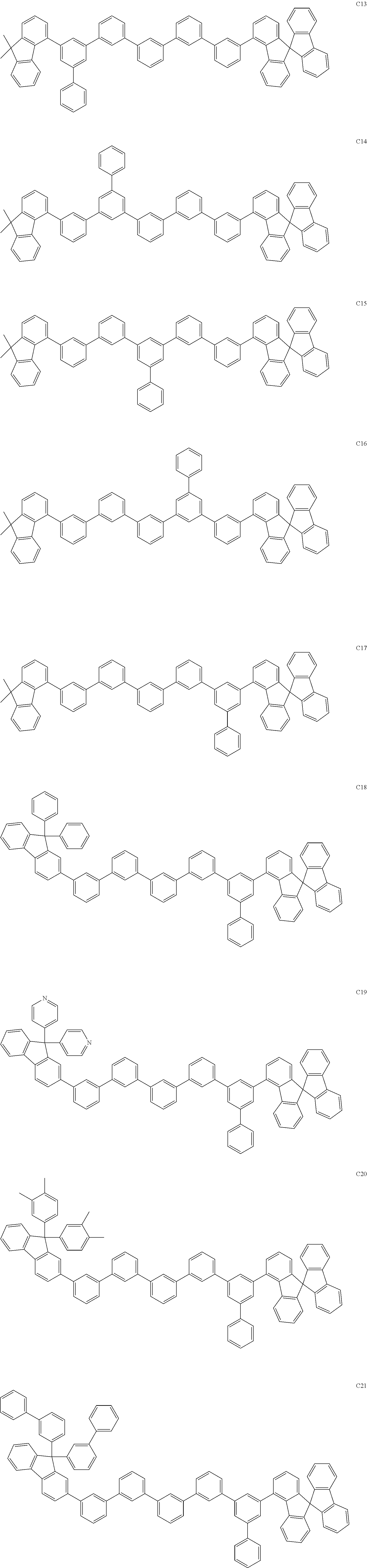

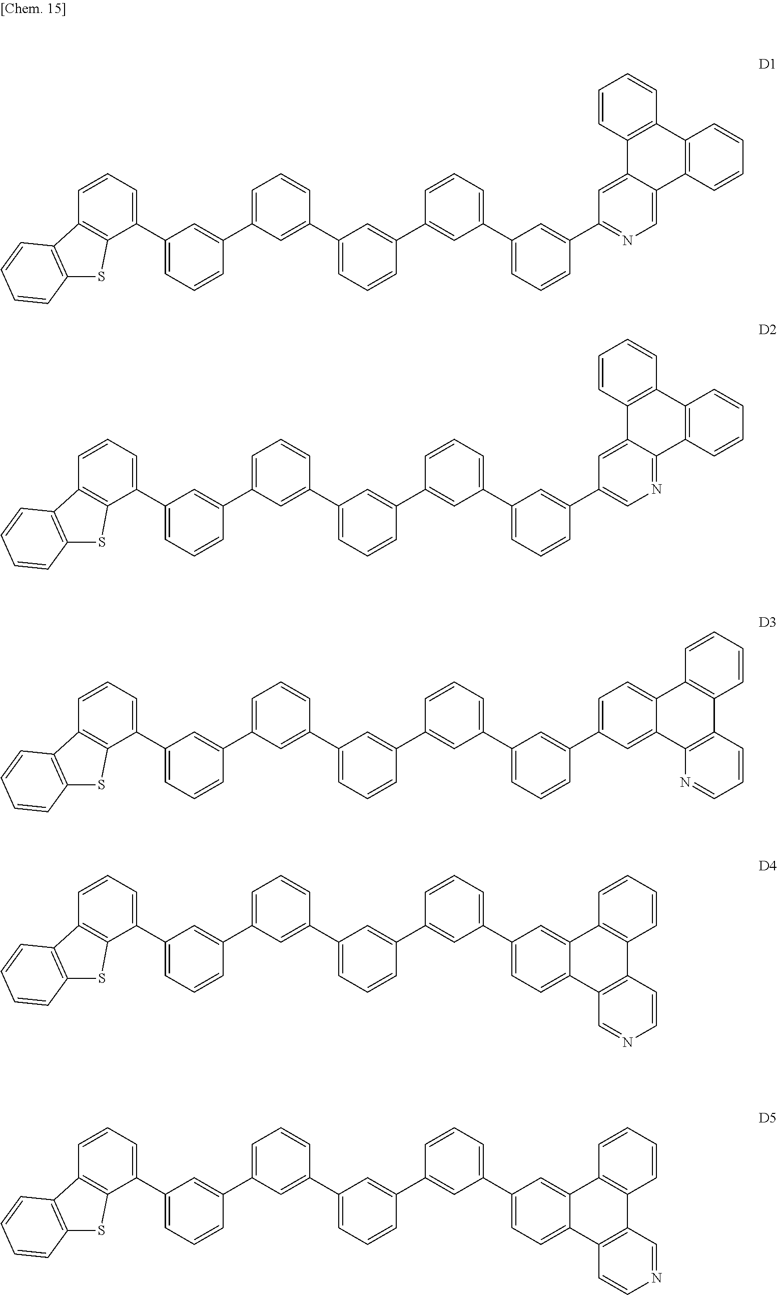

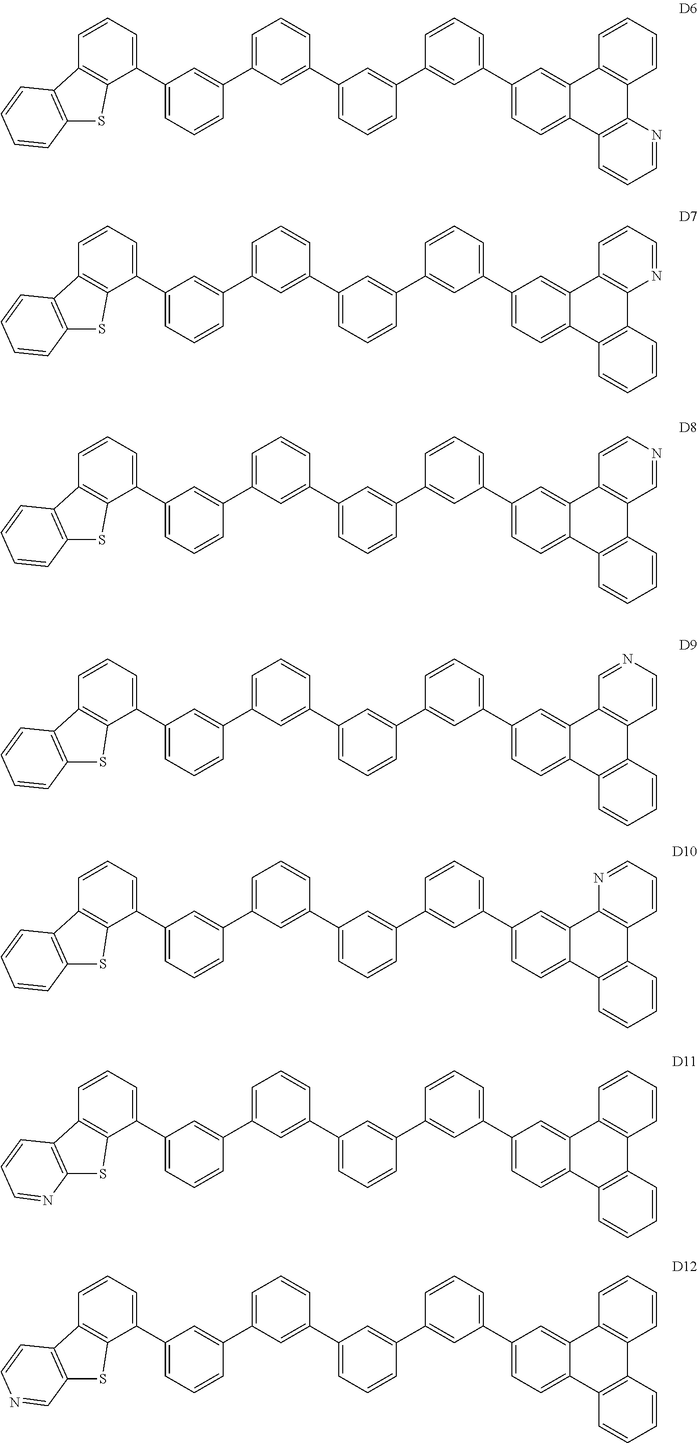

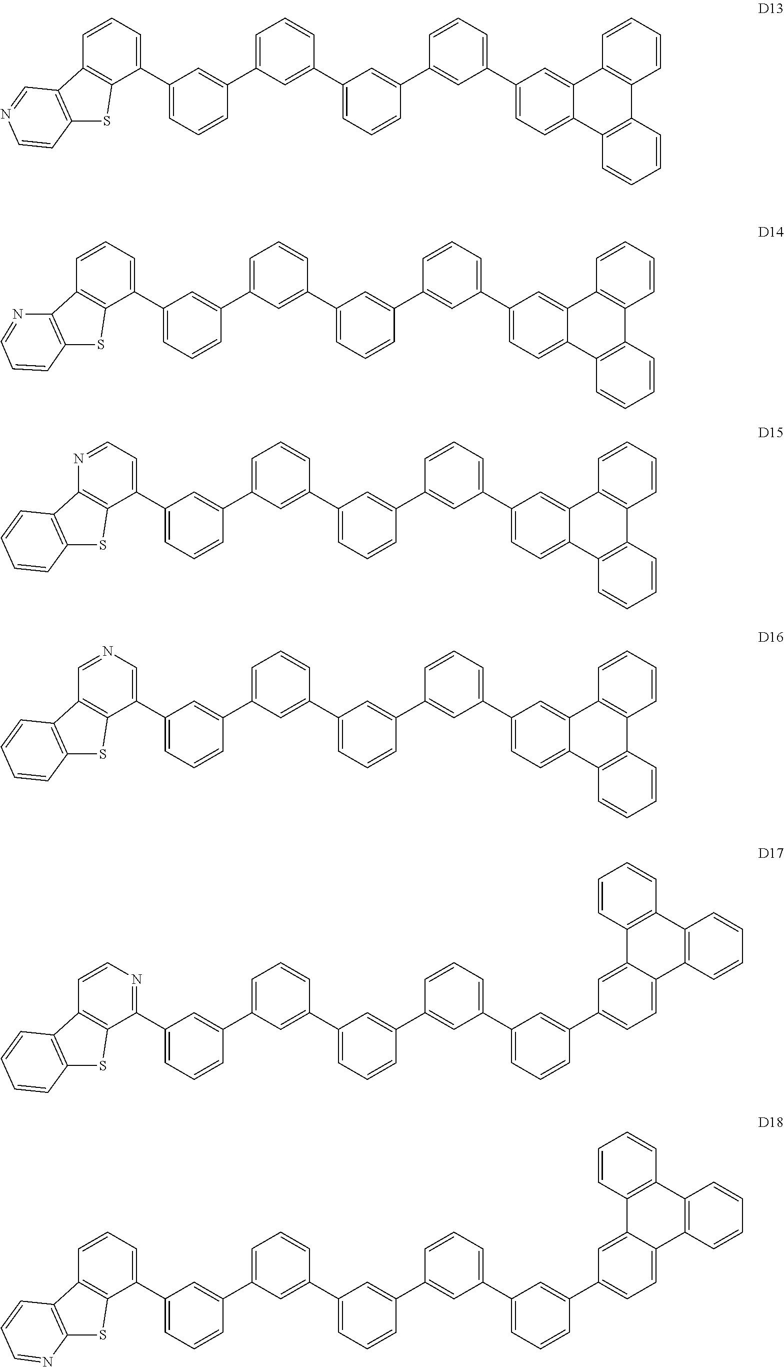

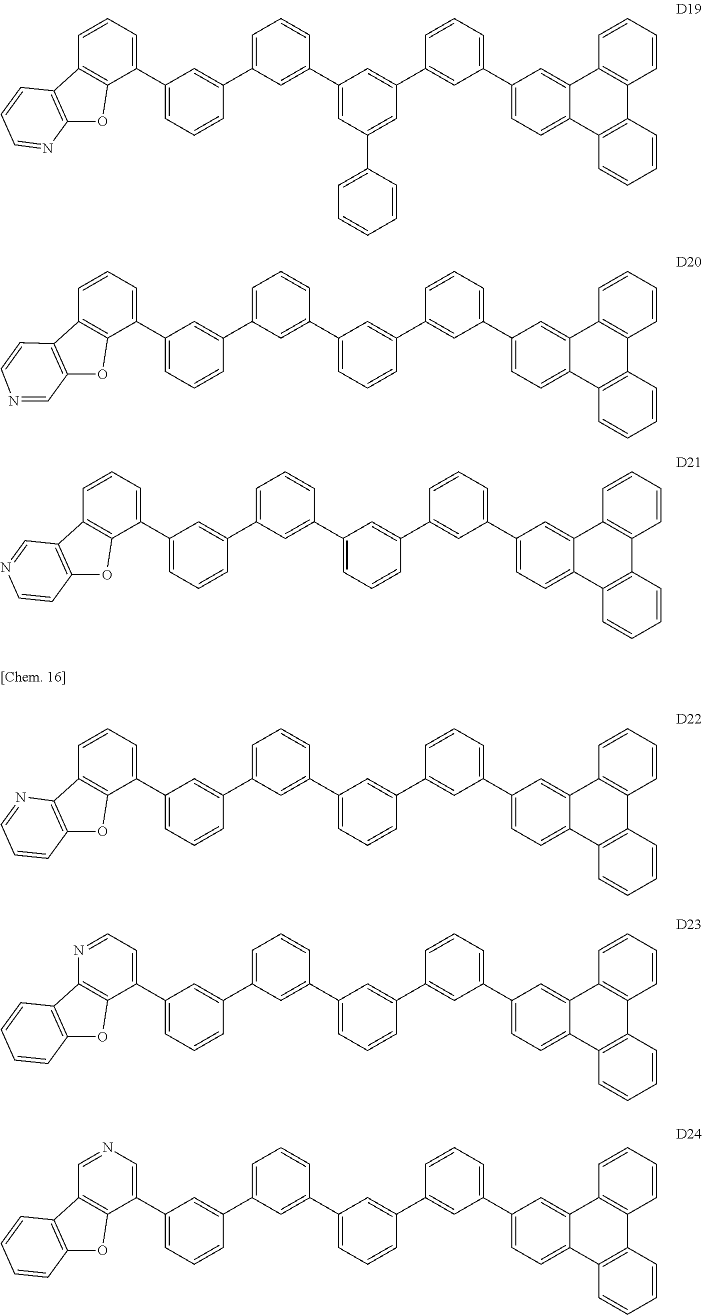

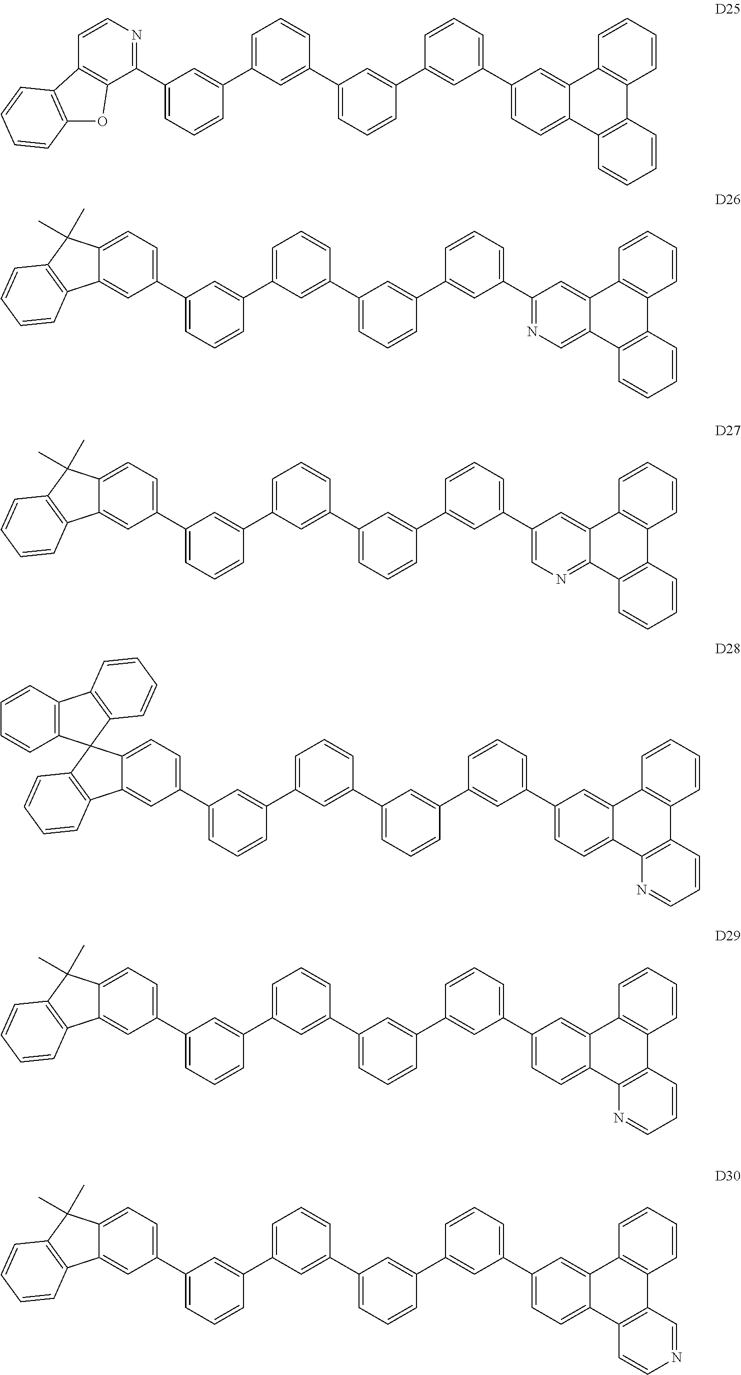

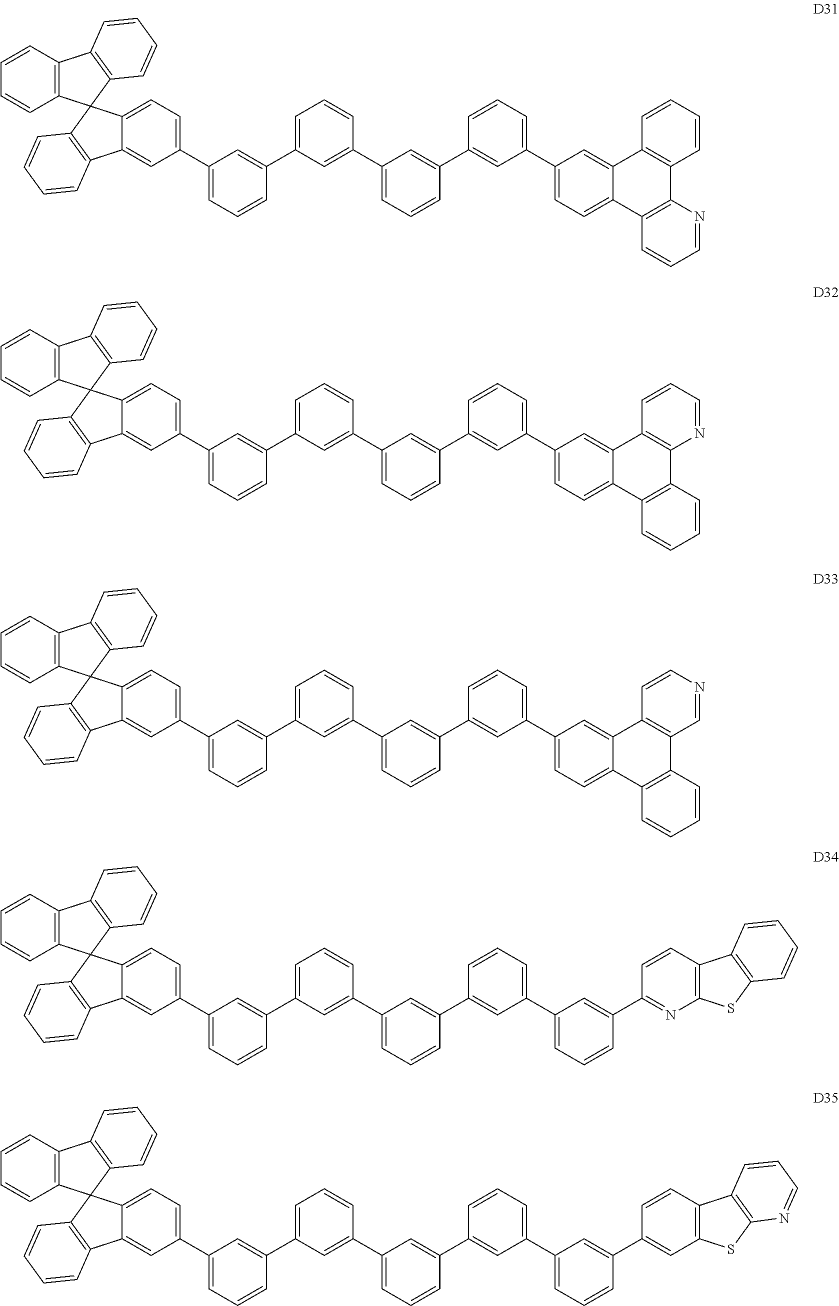

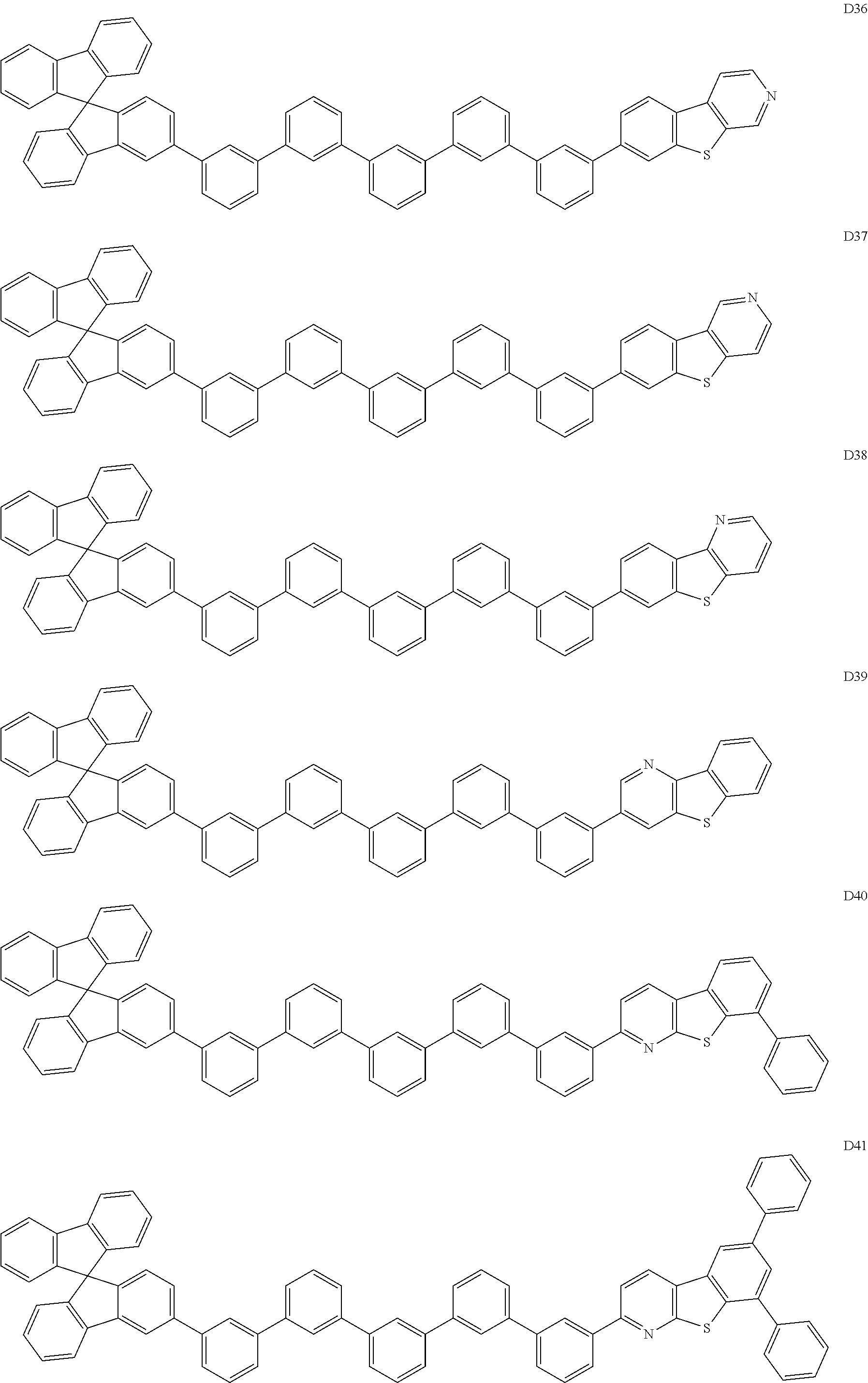

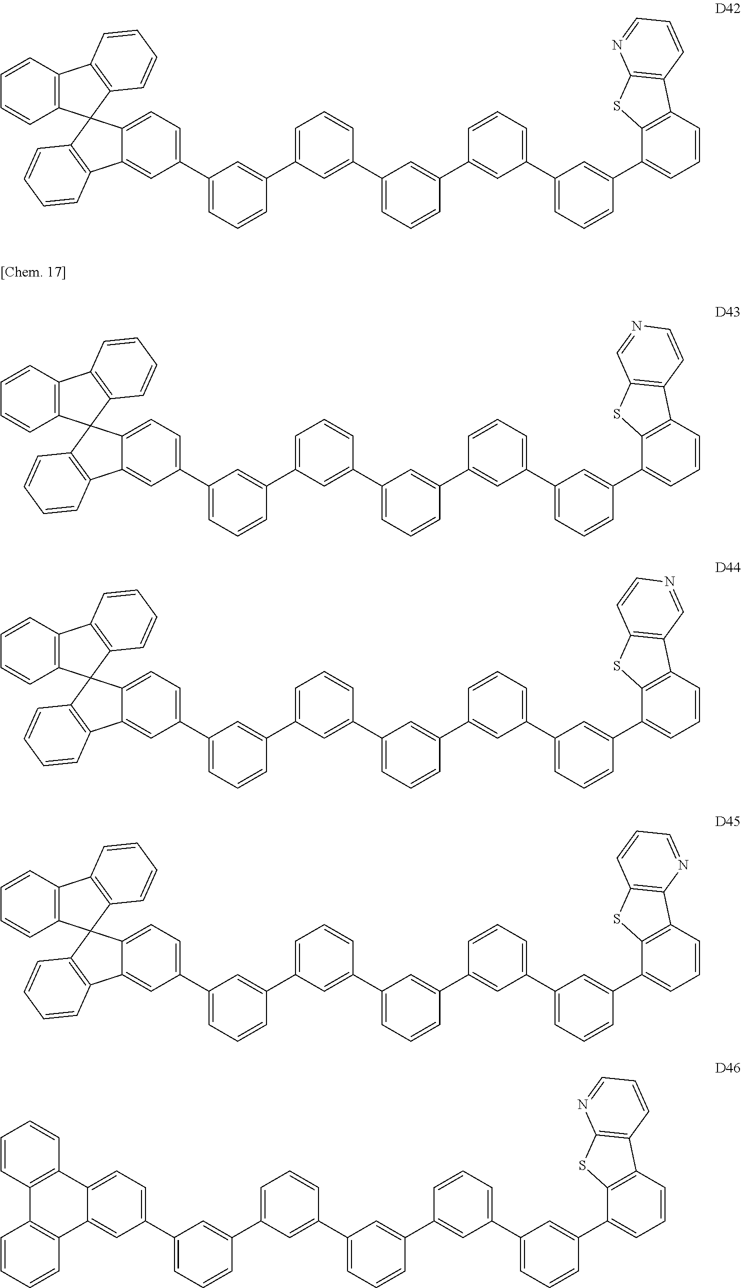

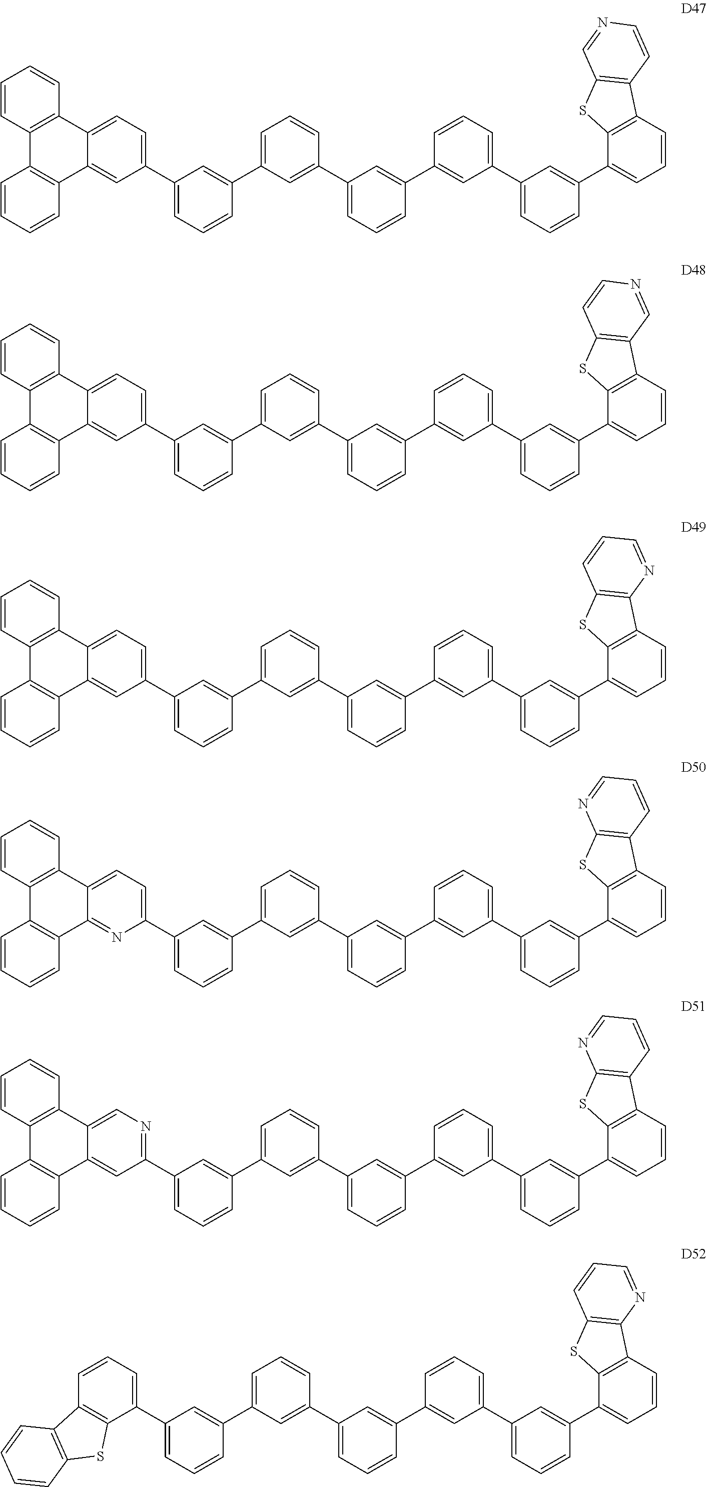

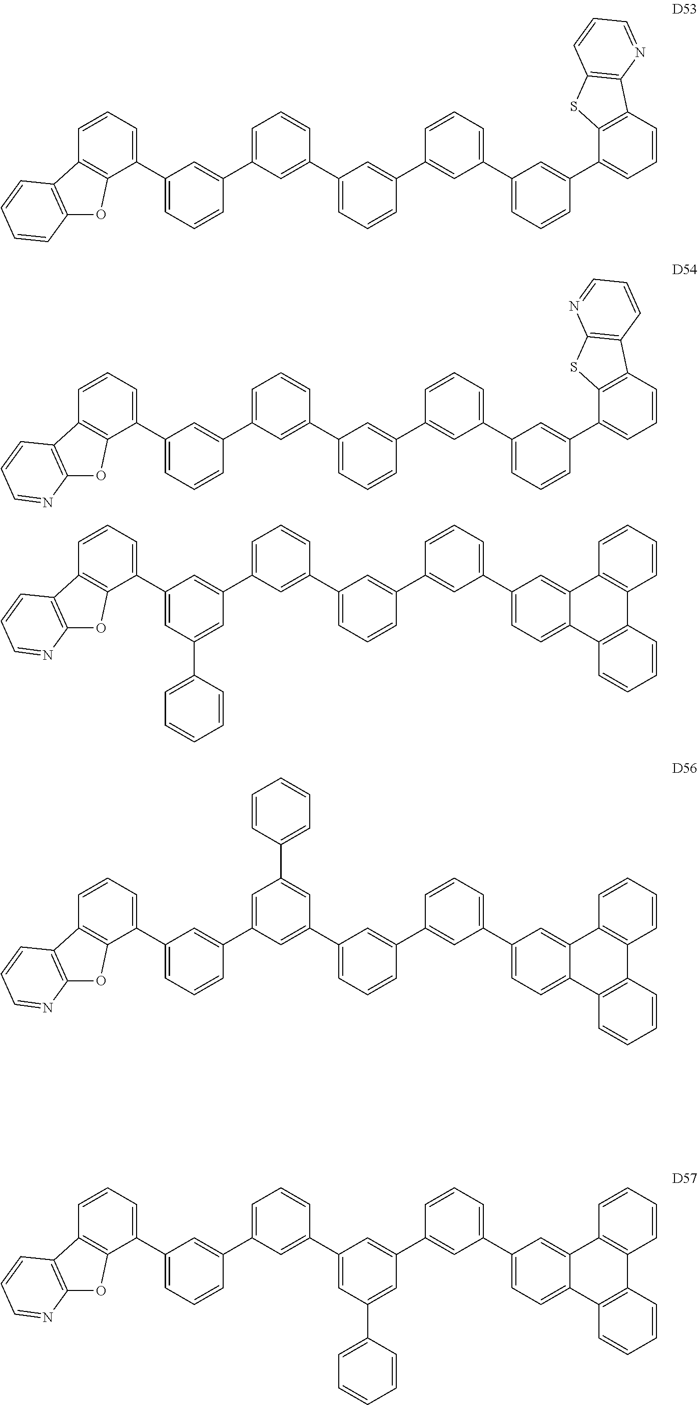

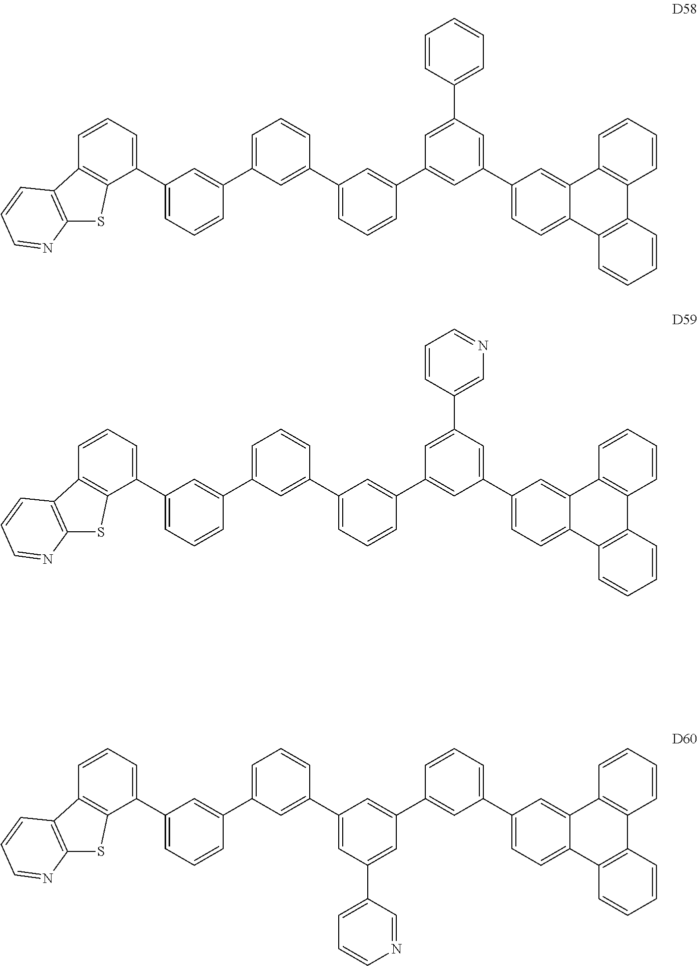 [SMILES: c1ccc(-c2cc(-c3cccc(-c4cccc(-c5cccc(-c6cccc7c6sc6ncccc67)c5)c4)c3)cc(-c3ccc4c5ccccc5c5ccccc5c4c3)c2)cc1 ; c1cncc(-c2cc(-c3cccc(-c4cccc(-c5cccc(-c6cccc7c6sc6ncccc67)c5)c4)c3)cc(-c3ccc4c5ccccc5c5ccccc5c4c3)c2)c1 ; c1cncc(-c2cc(-c3cccc(-c4cccc(-c5cccc6c5sc5ncccc56)c4)c3)cc(-c3cccc(-c4ccc5c6ccccc6c6ccccc6c5c4)c3)c2)c1]

Among these exemplary compounds, the exemplary compounds belonging to the A group are compounds in which Ar₁ and Ar₂ have no SP3 carbon. These compounds are particularly stable among the compounds represented by the general formula [1] because Ar₁ and Ar₂ have no SP3 carbon. In particular, preferably, one of Ar₁ and Ar₂ is a triphenylene skeleton, and the other is a dibenzothiophene skeleton, because this increases T1. Bonding at the 2-position of the triphenylene skeleton or at the 2-, 3-, or 4-position of the dibenzothiophene skeleton is more preferred from the perspective of binding stability.

Among the above exemplary compounds, the exemplary compounds belonging to the B group are compounds in which Ar₁ and Ar₂ have a dibenzofuran skeleton or a dibenzothiophene skeleton. In these compounds, Ar₁ and Ar₂ have an oxygen atom or a sulfur atom, and a large number of lone pairs of these atoms can enhance charge transport properties. Thus, the compounds are particularly easy to adjust the carrier balance. From the perspective of binding stability, the dibenzofuran skeleton or the dibenzothiophene skeleton is preferably bonded to the phenylene chain at any of the 2-, 3-, and 4-positions of the dibenzofuran skeleton or the dibenzothiophene skeleton.

Among the above exemplary compounds, the exemplary compounds belonging to the C group are compounds in which at least one of Ar₁ and Ar₂ has a fluorene skeleton. These compounds further have a substituent at the 9-position of the fluorene. Thus, the substituent in the direction perpendicular to the in-plane direction of the fluorene skeleton can particularly suppress the overlap of fused rings. Thus, the compounds have particularly high sublimability. The substituent at the 9-position of the fluorene is preferably an alkyl group with 1 to 4 carbon atoms or an aryl group with 6 to 12 carbon atoms. In particular, a bulkier substituent has a larger effect of suppressing the overlap of fused rings, and the fluorene more preferably has a phenyl group at the 9-position.

Among the above exemplary compounds, the exemplary compounds belonging to the D group are compounds in which at least one of Ar₁ and Ar₂ has an azine ring. These compounds contain a nitrogen atom in the fused ring, and a lone pair and high electronegativity of the nitrogen atom can enhance charge transport properties. Thus, the compounds are particularly easy to adjust the carrier balance.

### (2) Organic Light-Emitting Device

Next, an organic light-emitting device according to the present embodiment is described.

A specific device structure of the organic light-emitting device according to the present embodiment may be a multilayer device structure including an electrode layer and an organic compound layer shown in the following (a) to (f) sequentially stacked on a substrate. More specifically, the organic light-emitting device according to the present embodiment includes at least a pair of electrodes, a first electrode and a second electrode, and an organic compound layer between the electrodes. One of the first electrode and the second electrode may be a positive electrode, and the other may be a negative electrode. In any of the device structures, the organic compound layer always includes a light-emitting layer containing a light-emitting material.
(a) positive electrode/light-emitting layer/negative electrode
(b) positive electrode/hole transport layer/light-emitting layer/electron transport layer/negative electrode
(c) positive electrode/hole transport layer/light-emitting layer/electron transport layer/electron injection layer/negative electrode
(d) positive electrode/hole injection layer/hole transport layer/light-emitting layer/electron transport layer/negative electrode
(e) positive electrode/hole injection layer/hole transport layer/light-emitting layer/electron transport layer/electron injection layer/negative electrode
(f) positive electrode/hole transport layer/electron-blocking layer/light-emitting layer/hole-blocking layer/electron transport layer/negative electrode

These device structure examples are only very basic device structures, and the device structure of an organic light-emitting device according to the present invention is not limited to these device structures. For example, an insulating layer, an adhesive layer, or an interference layer may be provided at an interface between an electrode and an organic compound layer. An electron transport layer or a hole transport layer may have a multilayered structure having two layers with different ionization potentials. A light-emitting layer may have a multilayered structure having two layers each containing a different light-emitting material. Thus, a first light-emitting layer for emitting first light and a second light-emitting layer for emitting second light may be provided between a positive electrode and a negative electrode. An organic light-emitting device for emitting white light can be produced in which the white light is composed of first light and second light of different colors. In addition to such structures, various other layer structures can be employed.

In the present embodiment, the mode (device form) of extracting light from the light-emitting layer may be a so-called bottom emission mode of extracting light from an electrode on the substrate side or a so-called top emission mode of extracting light from the side opposite the substrate side. The mode may also be a double-sided extraction mode of extracting light from the substrate side and from the side opposite the substrate side.

Among the device structures shown in (a) to (f), the structure (f) is preferred due to the presence of both an electron-blocking layer (electron-stopping layer) and a hole-blocking layer (hole-stopping layer). Thus, the electron-blocking layer and the hole-blocking layer in (f) can securely confine carriers of both holes and electrons in the light-emitting layer. Thus, the organic light-emitting device has no carrier leakage and high light emission efficiency.

The organic light-emitting device according to the present embodiment contains an organic compound represented by the general formula [1] in the organic compound layer. The organic light-emitting device according to the present embodiment preferably contains an organic compound represented by the general formula [1] in the light-emitting layer. However, the present invention is not limited thereto, and it can be used as a constituent material of an organic compound layer other than the light-emitting layer constituting the organic light-emitting device according to the present embodiment. More specifically, it may be used as a constituent material of an electron transport layer, an electron injection layer, an electron-blocking layer, a hole transport layer, a hole injection layer, a hole-blocking layer, or the like.

In the organic light-emitting device according to the present embodiment, when the light-emitting layer contains an organic compound represented by the general formula [1], the light-emitting layer may be a layer composed of the organic compound represented by the general formula [1] and a first compound, which is another compound. For a light-emitting layer composed of an organic compound represented by the general formula [1] and another compound, an organic compound according to the present invention may be used as a host (hereinafter also referred to as a “host material”) or an assist (hereinafter also referred to as an “assist material”) of the light-emitting layer. When an organic compound according to the present invention is used as a host material, the first compound may be a guest (hereinafter also referred to as a “guest material”).

The host is a compound with the highest mass ratio among the compounds constituting the light-emitting layer. The guest is a compound that has a lower mass ratio than the host among the compounds constituting the light-emitting layer and that is a principal light-emitting compound. The assist material is a compound that has a lower mass ratio than the host among the compounds constituting the light-emitting layer and that assists the guest in emitting light. The assist material is also referred to as a second host. Alternatively, when the guest is referred to as a first compound, the assist can be referred to as a second compound.

The host is preferably a material with a higher LUMO (closer to the vacuum level) than the guest. This allows electrons supplied to the host of the light-emitting layer to be efficiently delivered to the guest and improves light emission efficiency. Furthermore, when an assist material is used in addition to the host and the guest, the host is preferably a material with a higher LUMO than the assist material (a material with a LUMO closer to the vacuum level). This allows electrons supplied to the host of the light-emitting layer to be efficiently delivered to the assist material, and the assist material can play a role in exciton recombination. This enables efficient energy transfer to the guest.

The lowest excited singlet energy and the lowest excited triplet energy of the host are denoted by Sh1 and Th1, respectively, and the lowest excited singlet energy and the lowest excited triplet energy of the guest are denoted by Sg1 and Tg1, respectively. Then, Sh1>Sg1 is preferably satisfied. Th1>Tg1 is also preferably satisfied. Furthermore, the energy Sa1 of S1 and the energy Ta1 of T1 of the assist material preferably satisfy Sa1>Sg1, more preferably Ta1>Tg1. Furthermore, Sh1>Sa1>Sg1 is still more preferably satisfied, and Th1>Ta1>Tg1 is still more preferably satisfied.

The present inventors conducted various studies and found that an organic light-emitting device with high light emission efficiency and durability can be produced when an organic compound represented by the general formula [1] is used as a host or an assist in a light-emitting layer, particularly as a host in the light-emitting layer.

An organic compound according to the present invention is more preferably used in a light-emitting layer in an organic light-emitting device under the following conditions. Two or more of the following conditions may be simultaneously satisfied.

(2-1) When the light-emitting layer contains an organic compound represented by the general formula [1] at a concentration of 30% by weight or more and 99% by weight or less of the entire light-emitting layer, the film has high thermal stability.

(2-2) When the first compound has a fused-ring structure in a ligand, the light emission efficiency is high.

(2-3) When the second compound has a structure of at least one of a carbazole skeleton, an azine skeleton, and a xanthone skeleton, the light emission efficiency is high.

These characteristics are described below.

(2-1) When the light-emitting layer contains an organic compound represented by the general formula [1] at a concentration of 30% by weight or more and 99% by weight or less of the entire light-emitting layer, the film has high thermal stability.

An organic compound according to the present invention easily maintains an amorphous state and is therefore a material suitable for a host material for a light-emitting layer. When an organic compound according to the present invention is used in a light-emitting layer, the concentration of the organic compound according to the present invention is preferably 30% by weight or more and 99% by weight or less of the entire light-emitting layer. The concentration of an organic compound according to the present invention is preferably 50% by weight or more and 99% by weight or less, more preferably 70% by weight or more and 99% by weight or less, of the entire light-emitting layer. An organic compound according to the present invention easily maintains an amorphous state, is unlikely to crystallize, and therefore has long device life even when the concentration thereof is 99% by weight of the entire light-emitting layer.

From the perspective of improving the thermal stability of a light-emitting layer, an organic compound according to the present invention may also be used as an assist material. When used as an assist material, an organic compound according to the present invention can be used at a concentration of 30% by mass or more and 50% by mass or less of the entire light-emitting layer.

(2-2) When the first compound has a fused-ring structure in a ligand, the light emission efficiency is high.

In an organic compound according to the present invention, one of Ar₁ and Ar₂ is a substituted or unsubstituted aryl group, and the other is a substituted or unsubstituted heterocyclic group. More specifically, one of Ar₁ and Ar₂ is a substituted or unsubstituted tricyclic or polycyclic aryl group, and the other is a substituted or unsubstituted tricyclic or polycyclic heterocyclic group. Thus, in a guest material used together with an organic compound according to the present invention in a light-emitting layer, a ligand preferably has a fused-ring structure. More specifically, a compound with a structure in which the n-conjugation of the ligand is further extended is preferred, and a compound with a tricyclic or polycyclic fused-ring structure is more preferred. This is because when an organic compound according to the present invention and the guest material have a structure with high planarity, the structure with high planarity of the organic compound according to the present invention and the structure with high planarity of the guest material can be close to each other by interaction. More specifically, at least one of Ar₁ and Ar₂ of an organic compound according to the present invention is likely to be close to the ligand of the guest material. This can be expected to decrease the intermolecular distance between an organic compound according to the present invention and the guest material.

It is known that triplet energy utilized in a phosphorescent device is transferred by the Dexter mechanism. In the energy transfer by the Dexter mechanism, energy is transferred by contact between molecules. More specifically, the intermolecular distance between a host material and a guest material is shortened for efficient energy transfer from the host material to the guest material.

As described above, the use of a highly planar compound with a fused-ring structure in a ligand as a guest material shortens the intermolecular distance between an organic compound according to the present invention and the guest material. This is likely to cause energy transfer from the organic compound according to the present invention to the guest material by the Dexter mechanism.

Consequently, an organic light-emitting device with high light emission efficiency can be provided.

More specifically, the first compound can be represented by the general formula [2].

Ir(L)q(L′)r(L″)s [2]

In the general formula [2], L, L′, and L″ independently denote a different bidentate ligand.

q denotes an integer in the range of 1 to 3, and r and s each denote an integer in the range of 0 to 2, provided that q+r+s=3. When r is 2, a plurality of L′ may be the same or different. When s is 2, a plurality of L″ may be the same or different.

The substructure Ir(L)q is represented by one of the following general formulae [Ir-1] to [Ir-12].

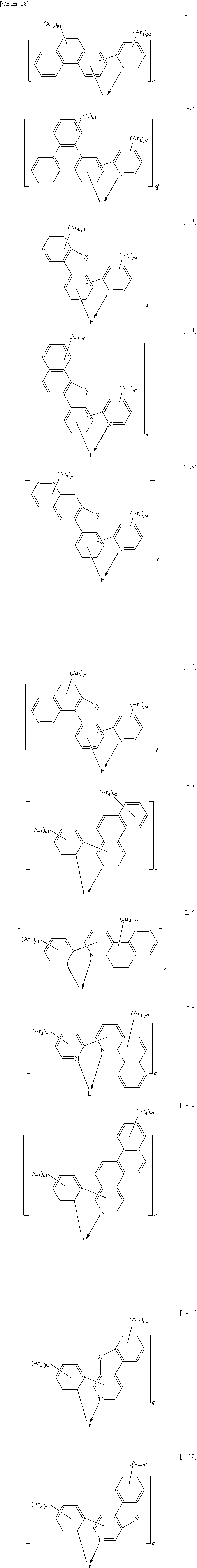

In general formulae [Ir-1] to [Ir-12], Ar₃ and Ar₄ denote a deuterium atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted silyl group, or a cyano group. More specifically, Ar₃ preferably denotes a deuterium atom, a fluorine atom, an alkyl group with 1 to 6 carbon atoms, an alkoxy group with 1 to 4 carbon atoms, an aryl group with 6 to 10 carbon atoms, a silyl group substituted with an alkyl group, or a cyano group, more preferably a methyl group, a tert-butyl group, or a phenyl group.

X is selected from an oxygen atom, a sulfur atom, C(R₁) (R₂), and NR₃.

R₁ to R₃ are each independently selected from a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group. R₁ and R₂ may be bonded together to form a ring. More specifically, R₁ to R₃ preferably denote an alkyl group with 1 to 3 carbon atoms or a phenyl group, more preferably a methyl group.

p₁ and p₂ each denote an integer in the range of 0 to 4.

More specifically, the first compound more preferably has a triphenylene skeleton, a phenanthrene skeleton, a fluorene skeleton, a benzofluorene skeleton, a dibenzofuran skeleton, a dibenzothiophene skeleton, a benzoisoquinoline skeleton, or a naphthoisoquinoline skeleton in a ligand. By using the first compound with at least one of these skeletons in a ligand, an organic compound according to the present embodiment can provide an organic light-emitting device with higher light emission efficiency.

Specific examples of the first compound according to the present embodiment are shown below. However, the present invention is not limited thereto. In the following structural formulae, when two bonds between a ligand and an iridium atom are represented by a solid line, one of the bonds may be a covalent bond, and the other bond may be a coordinate bond. On the other hand, when there are a solid line and a dotted line, the solid line may be a covalent bond, and the dotted line may be a coordinate bond. [Chem. 19]

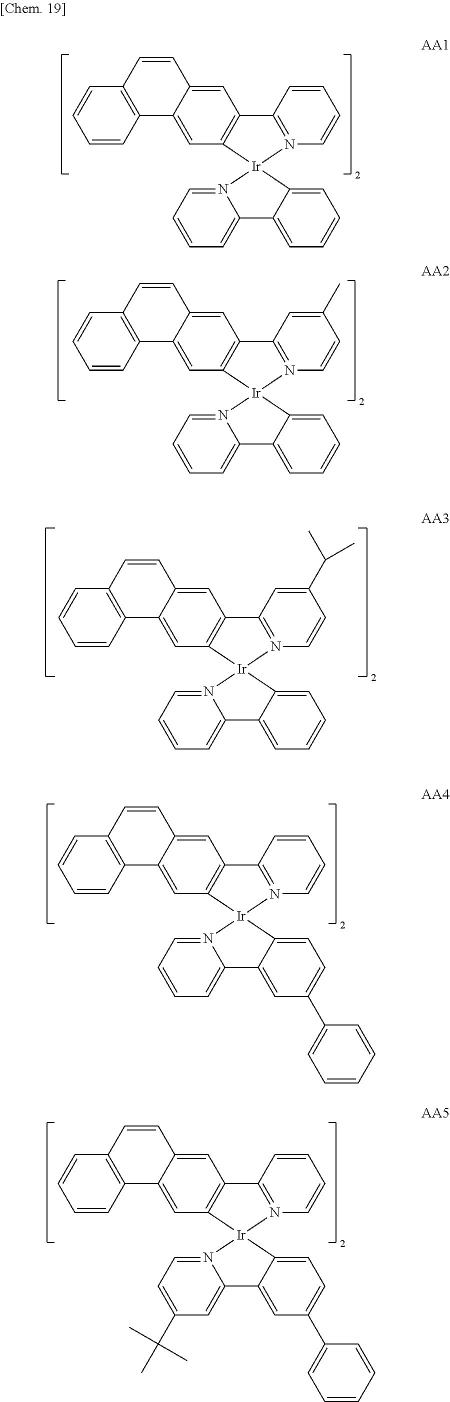

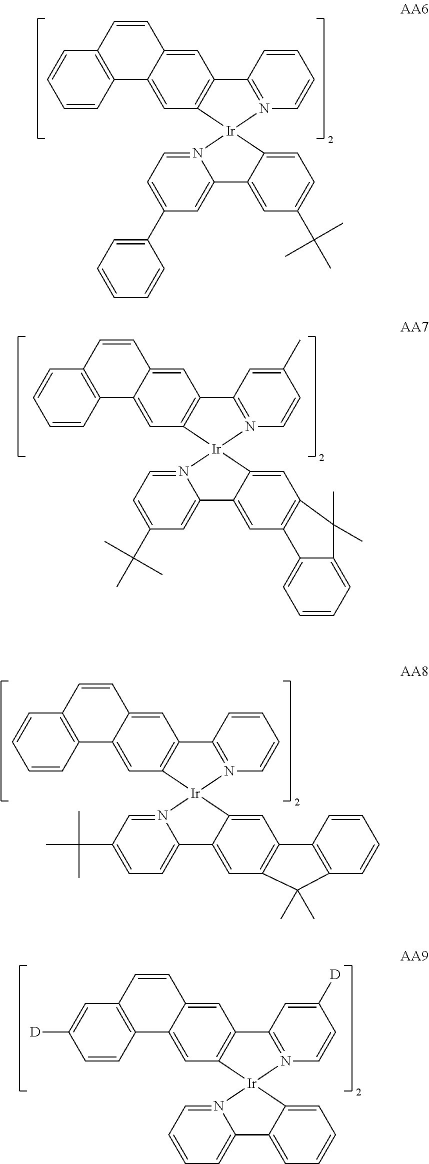

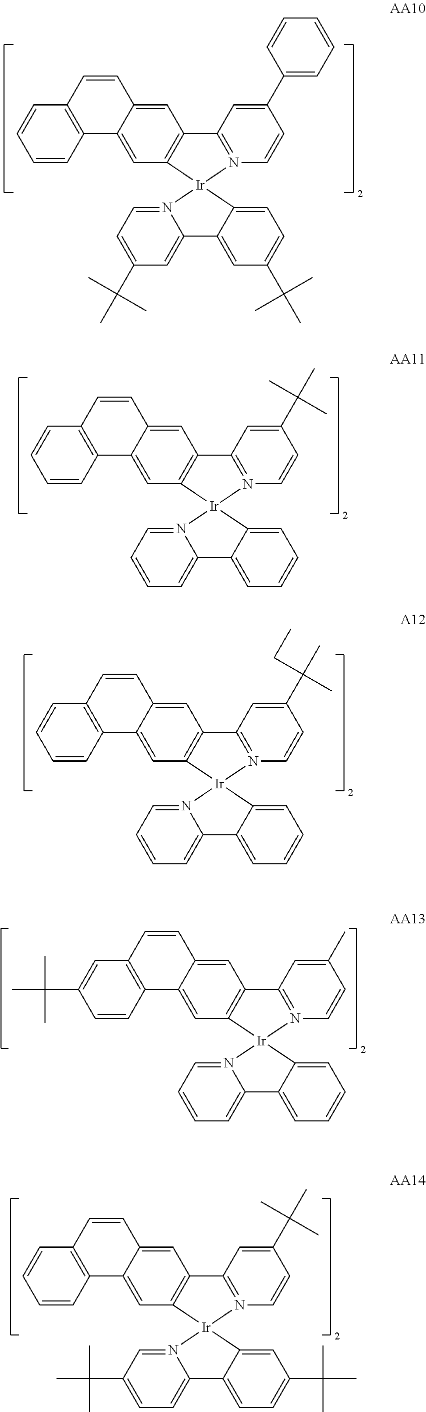

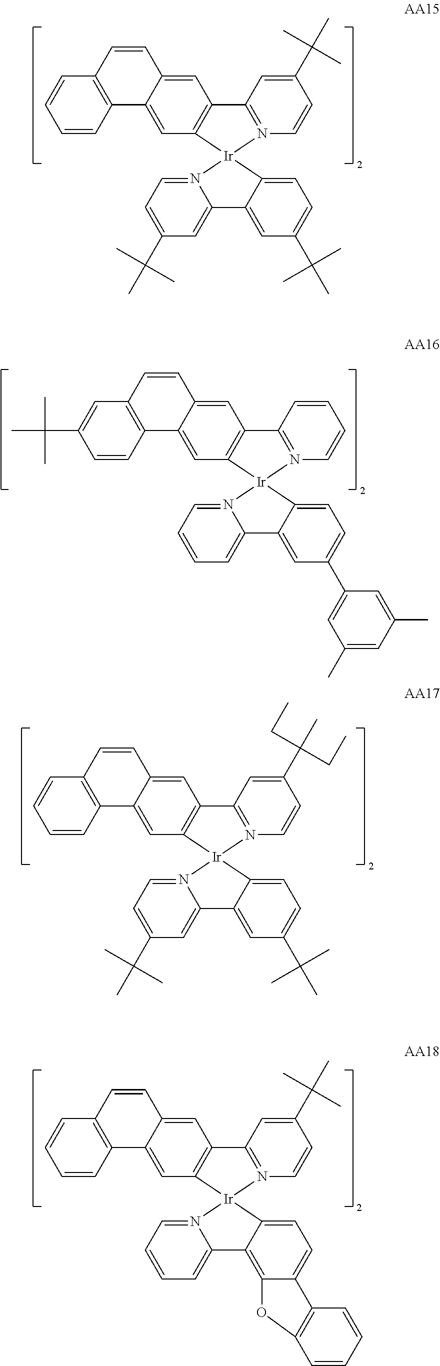

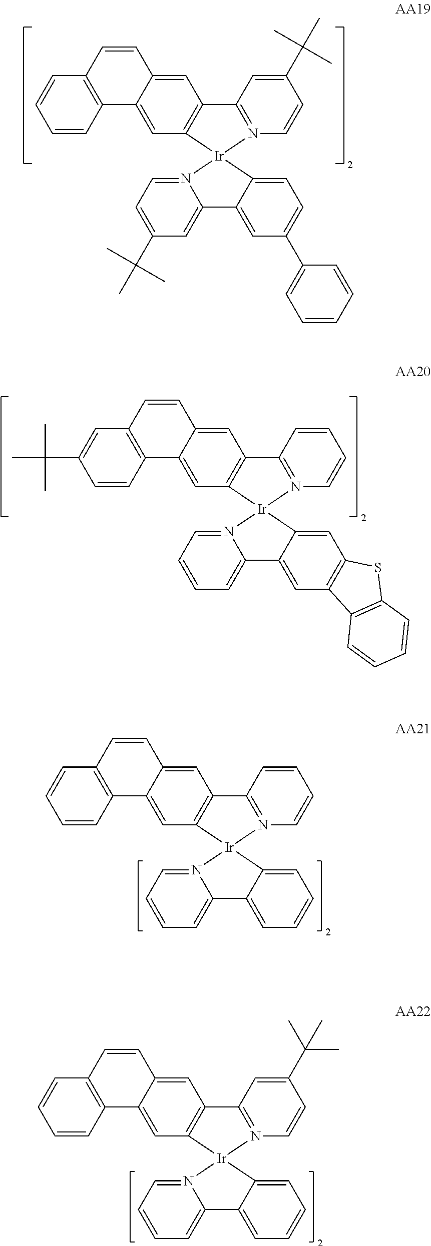

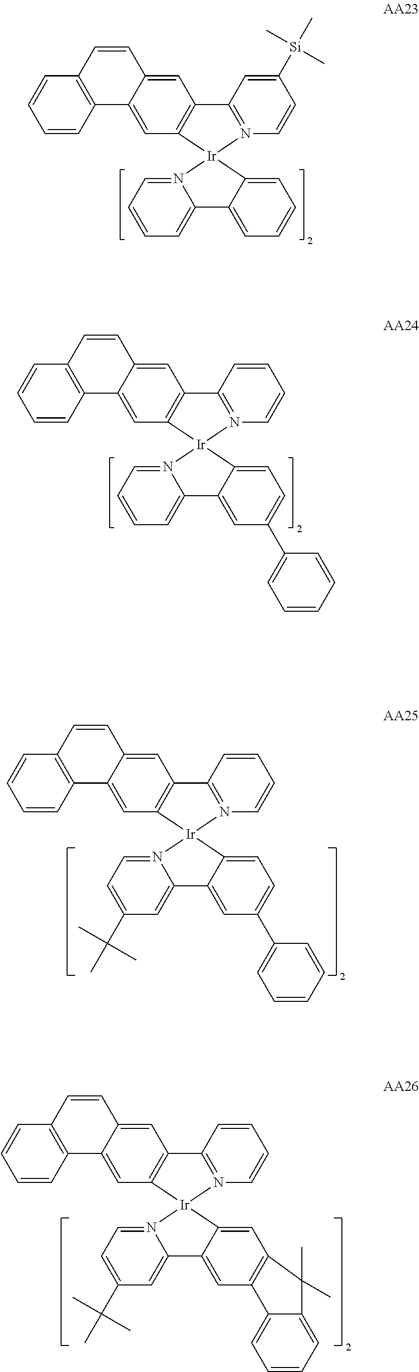

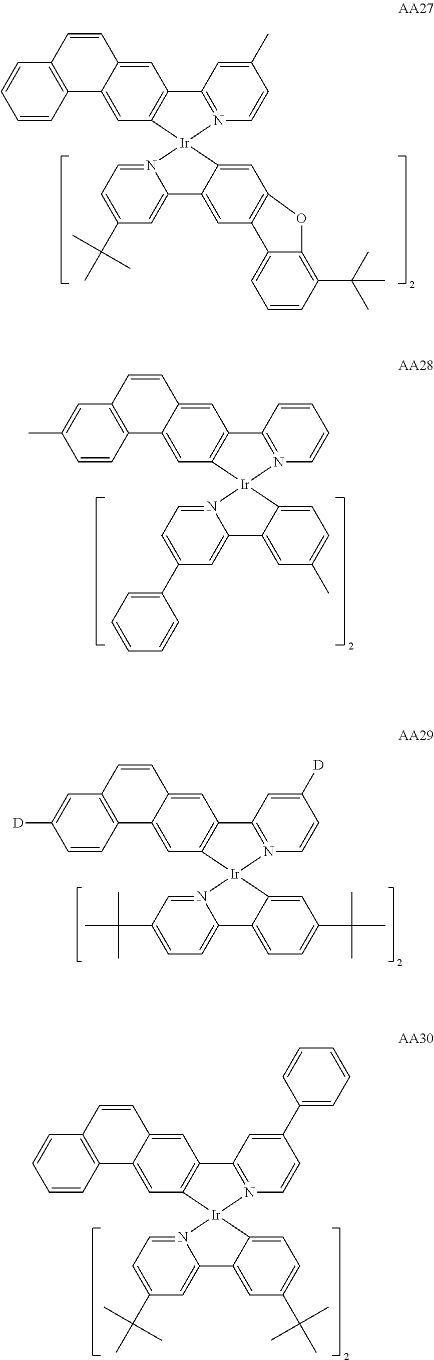

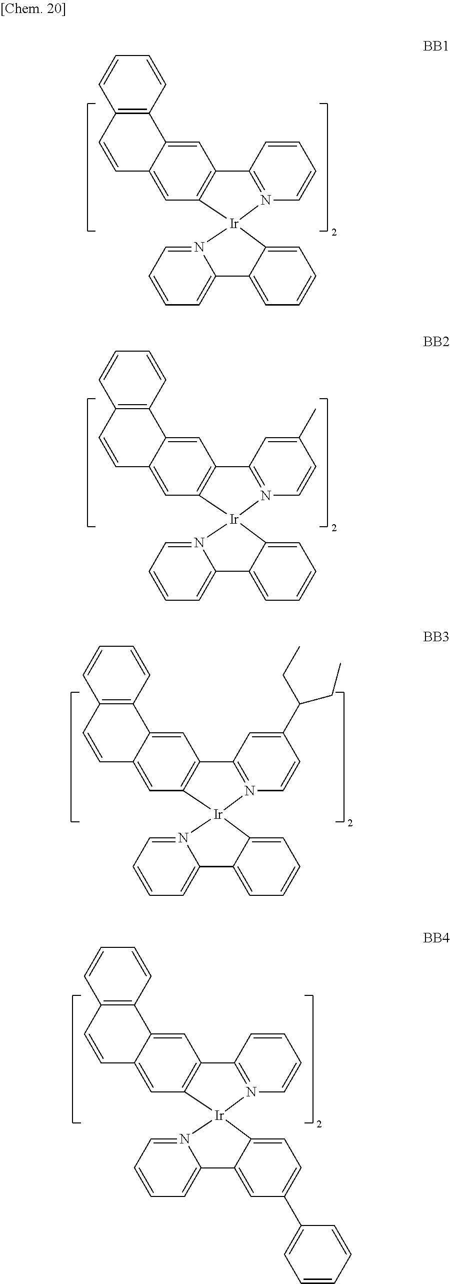

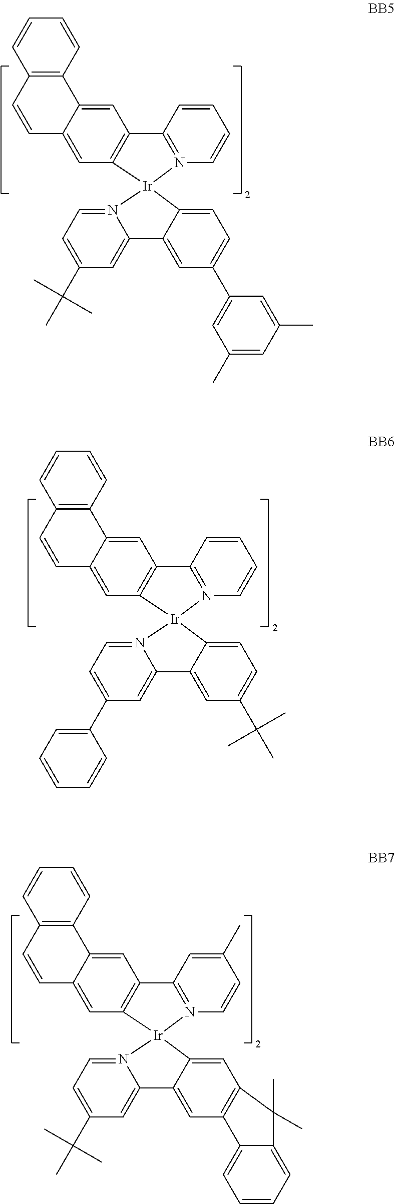

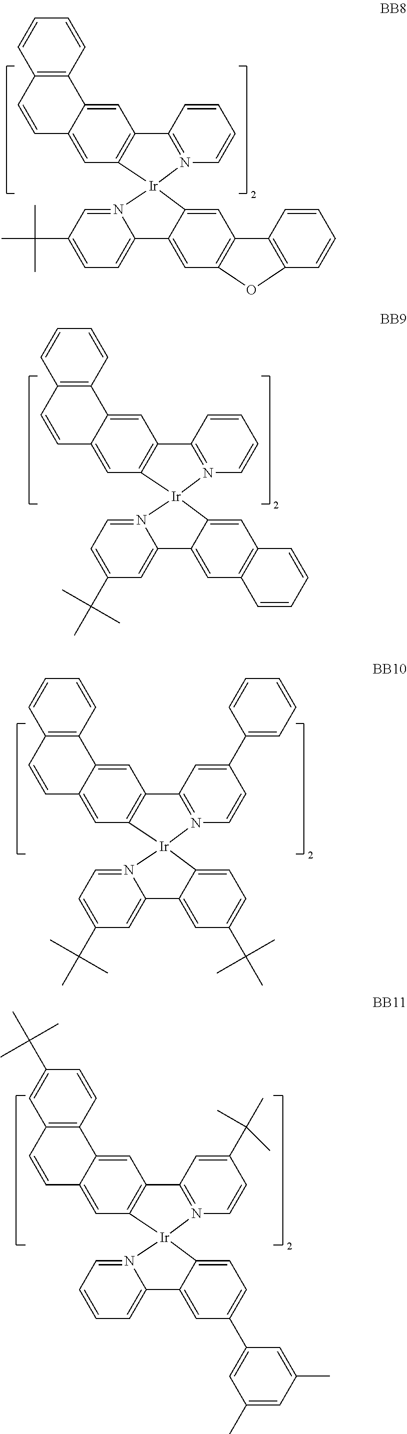

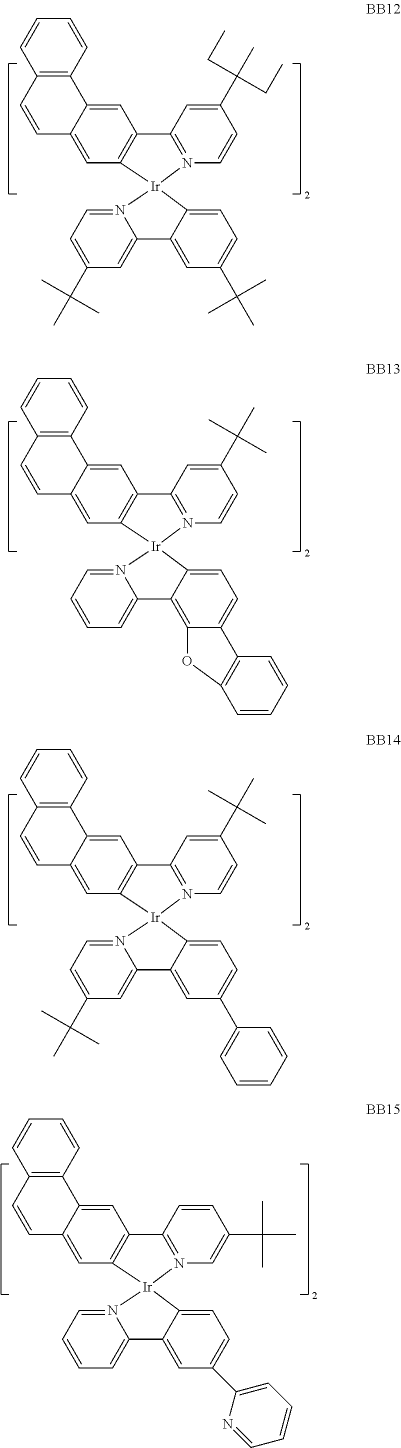

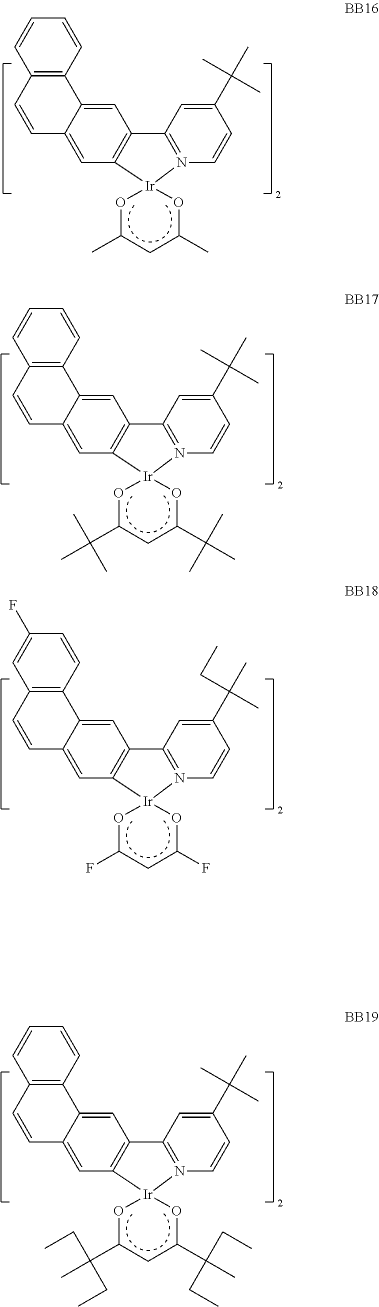

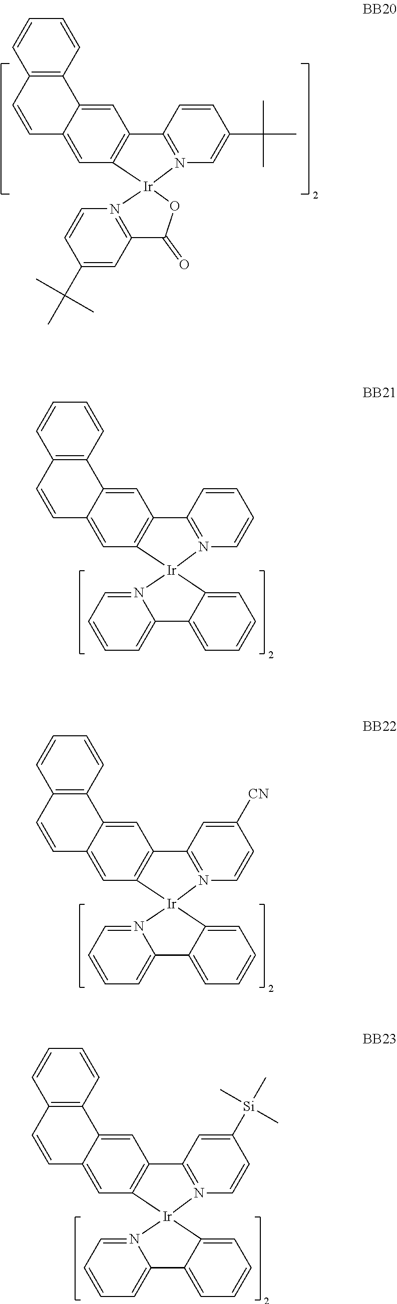

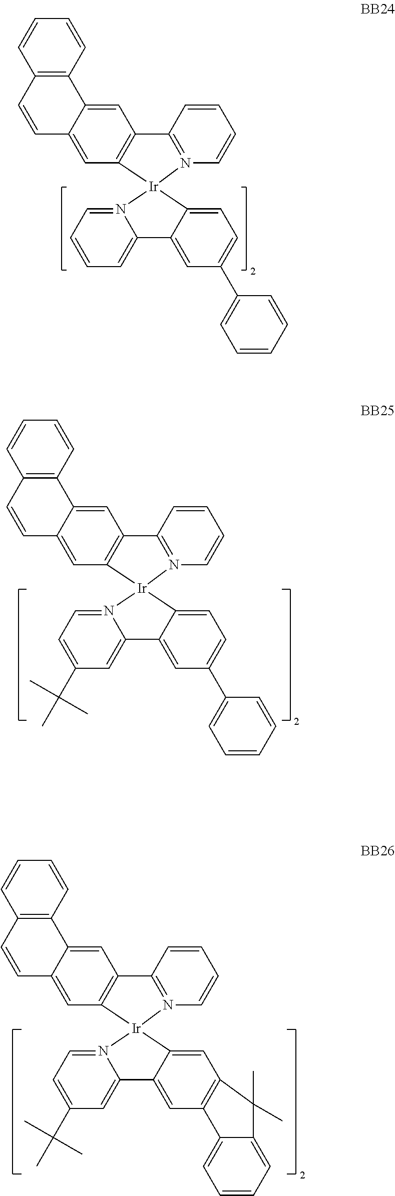

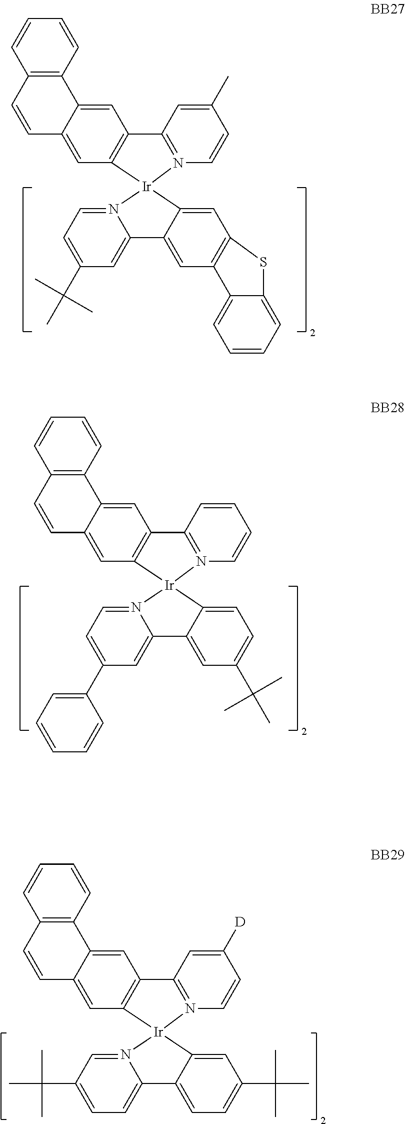

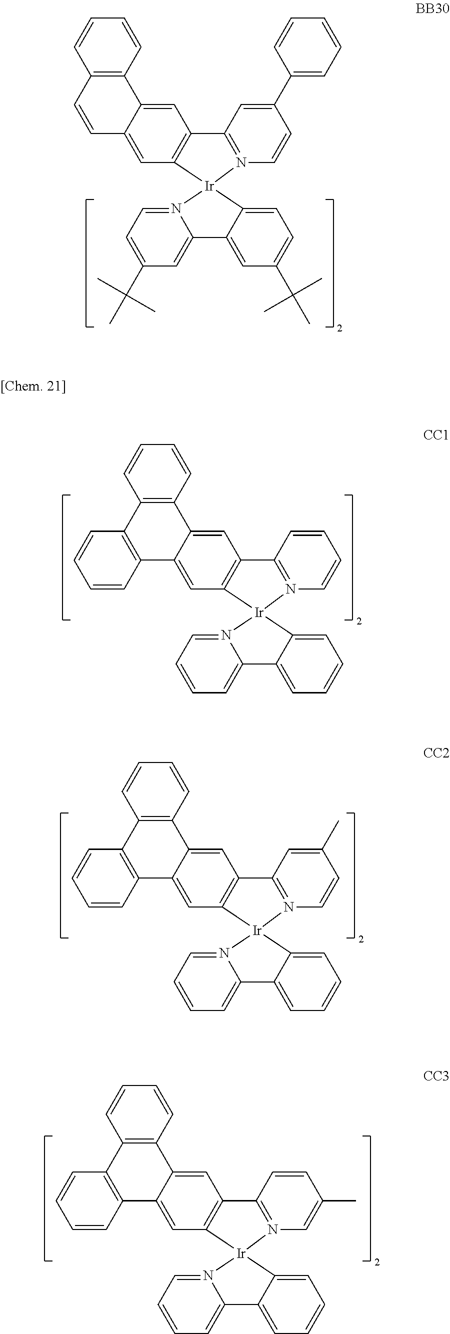

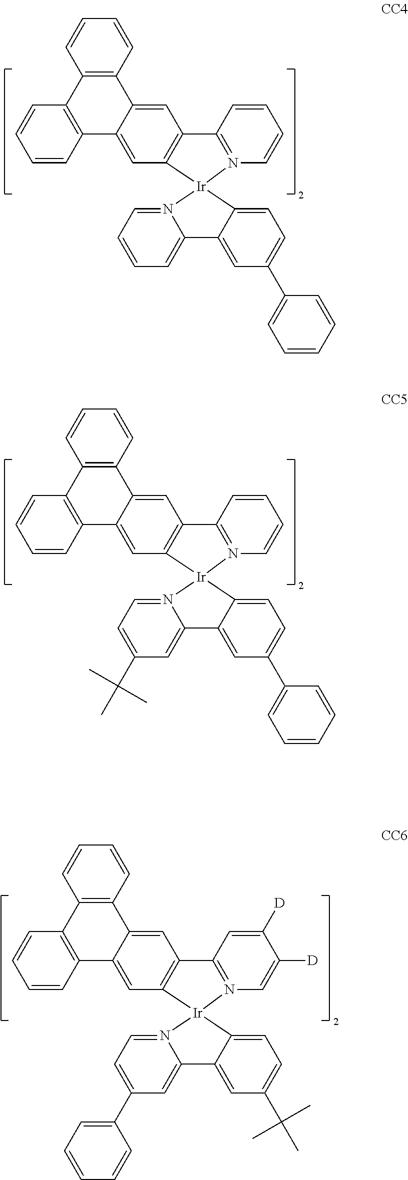

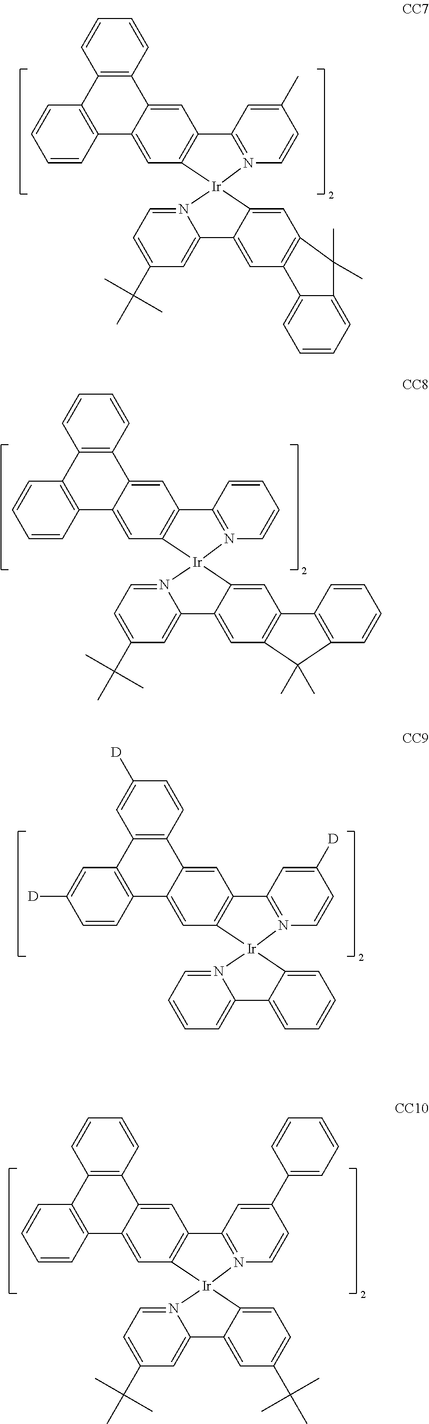

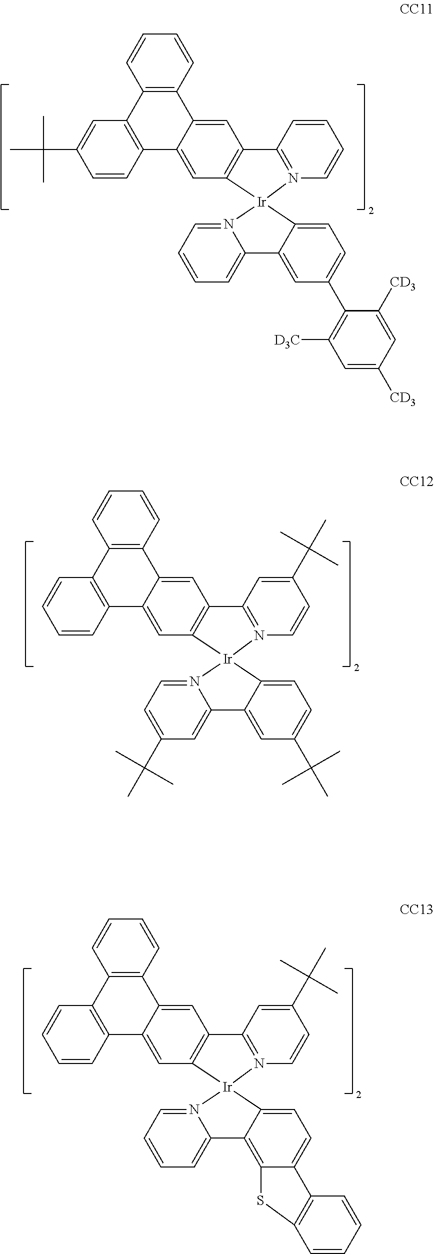

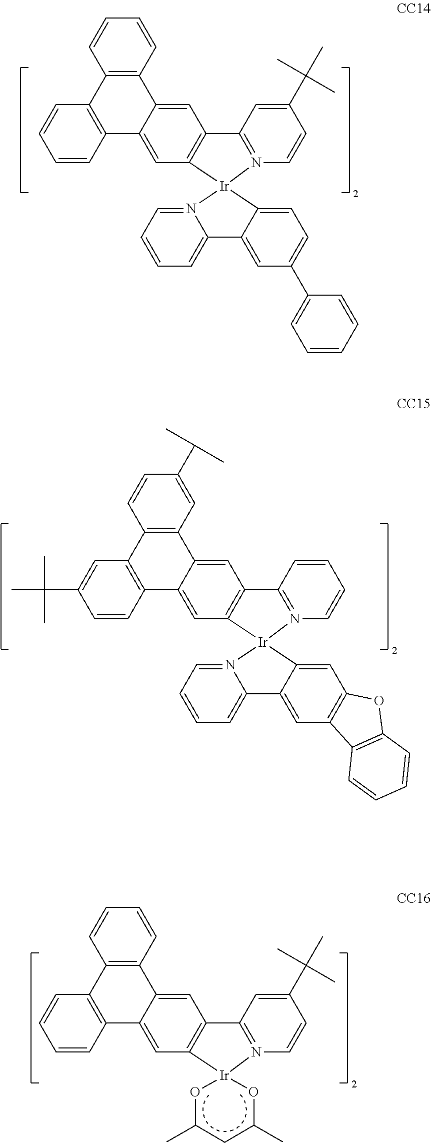

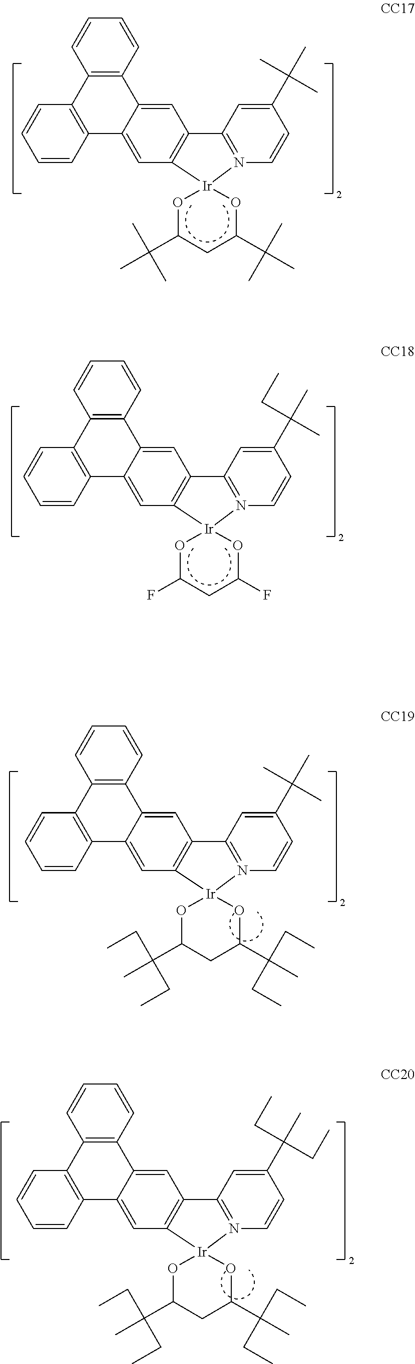

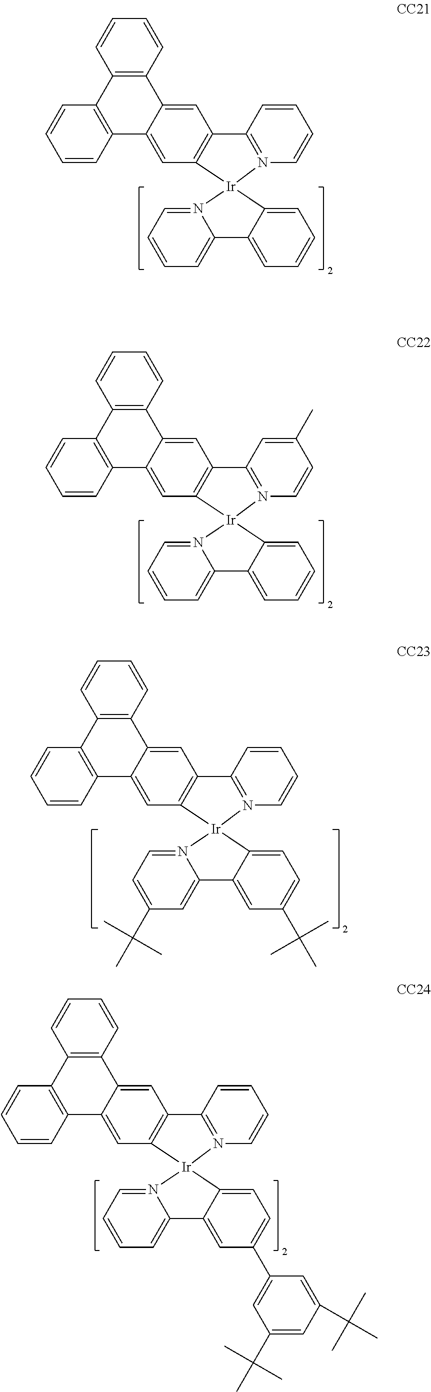

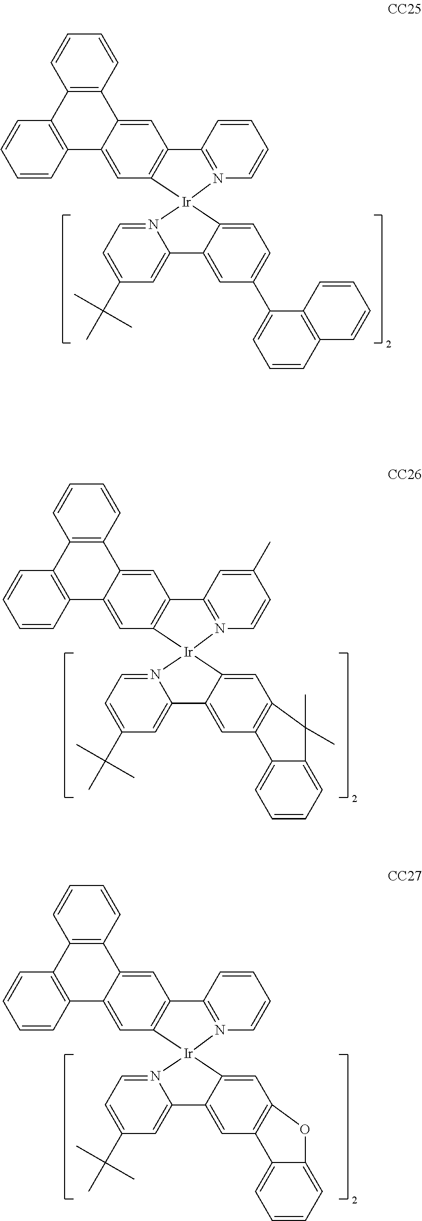

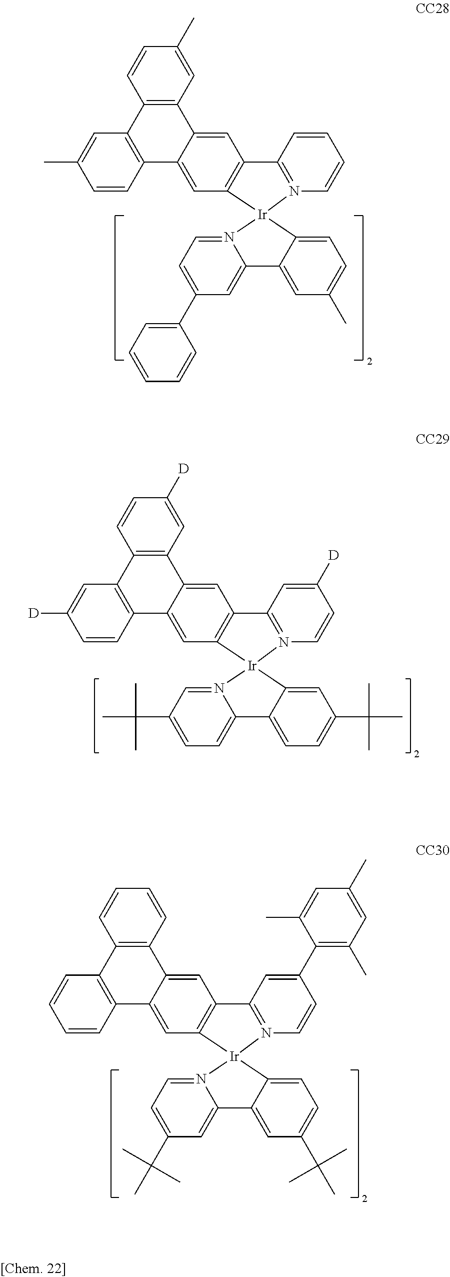

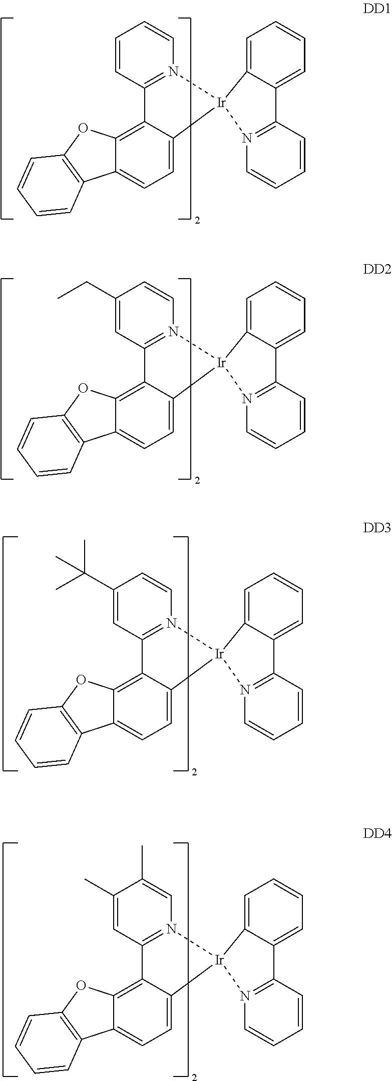

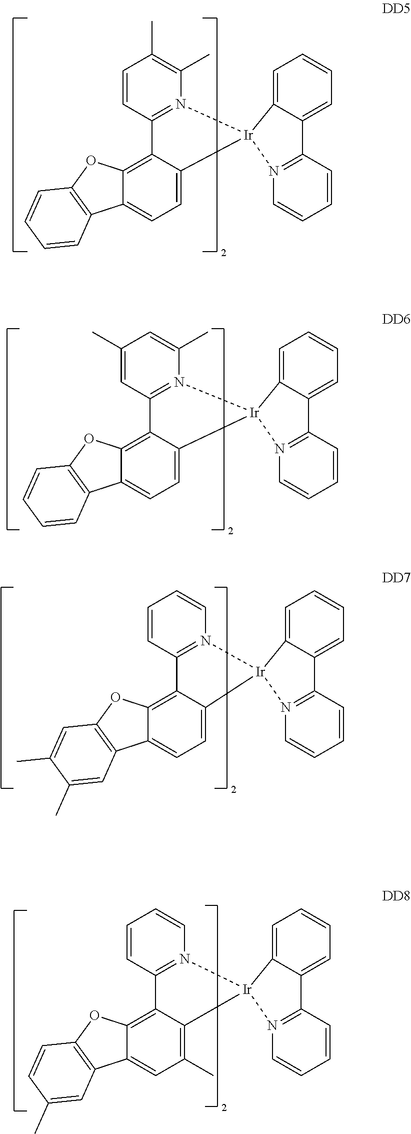

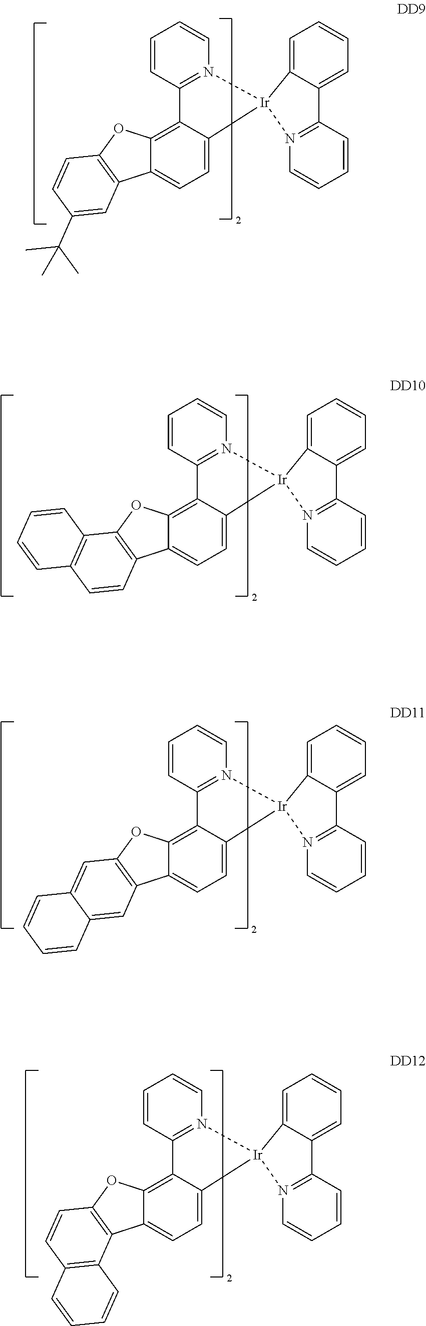

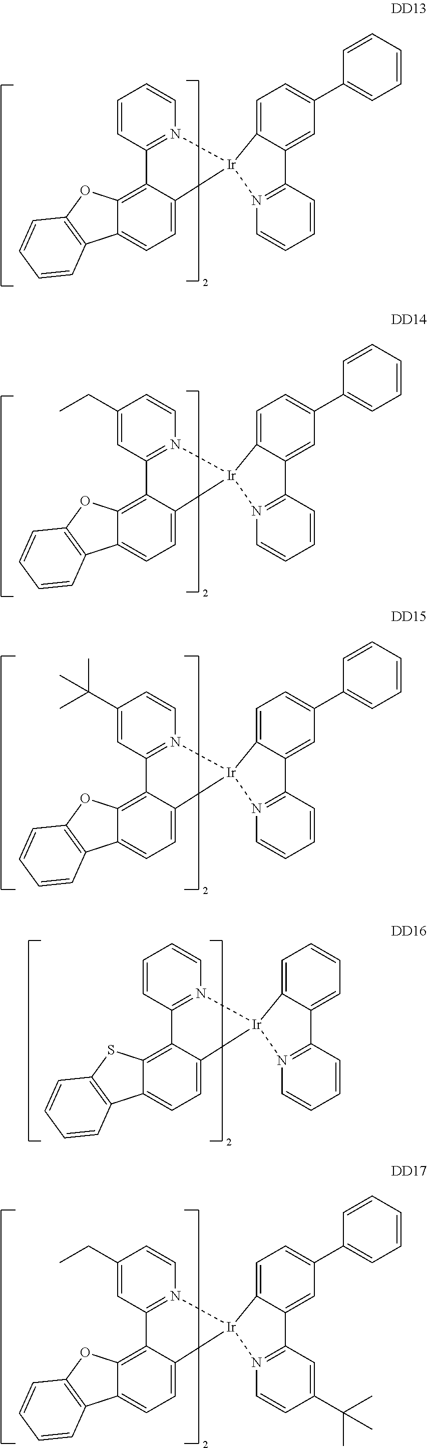

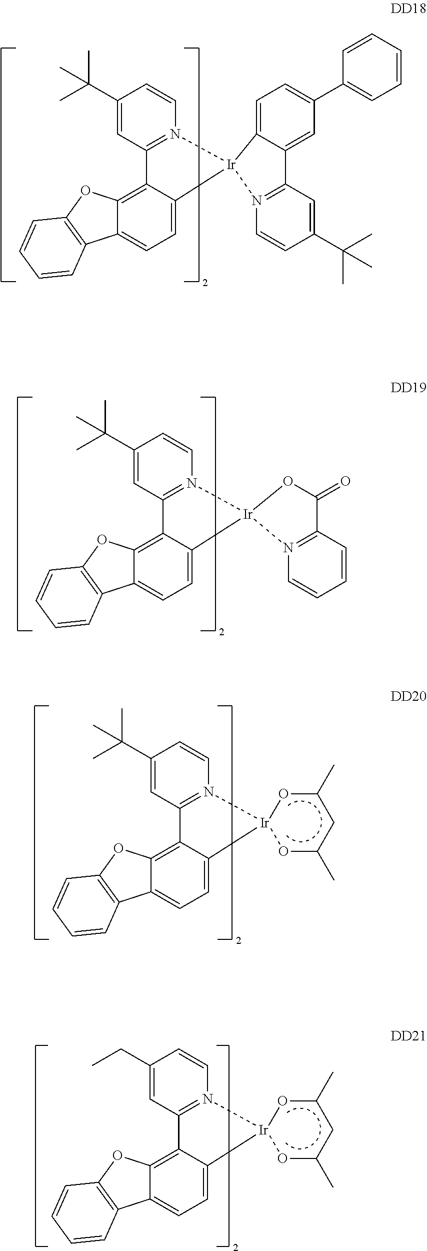

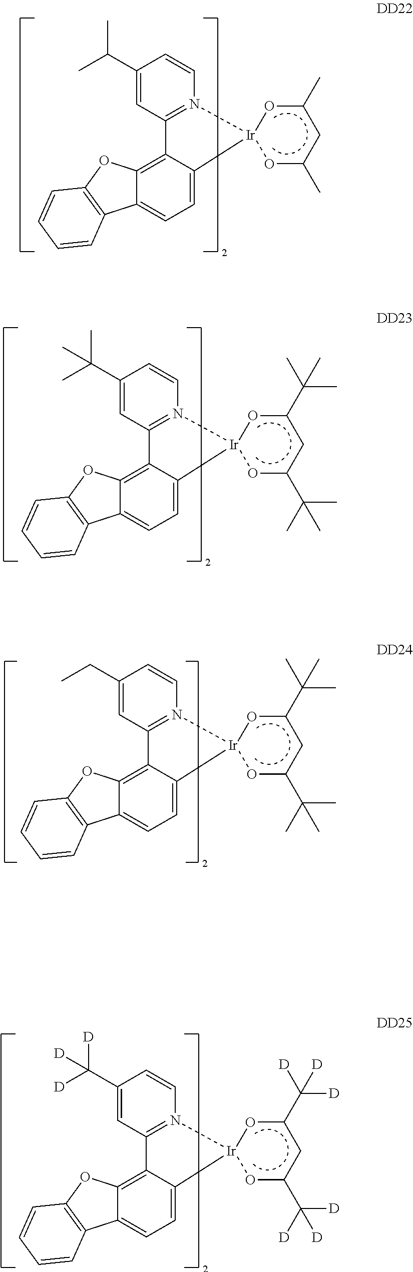

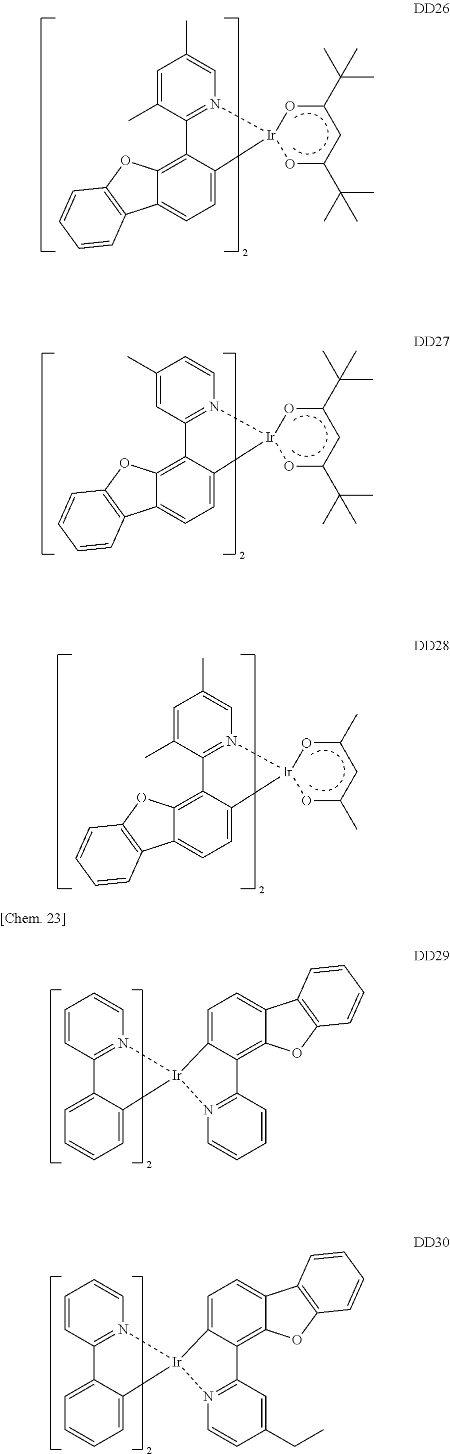

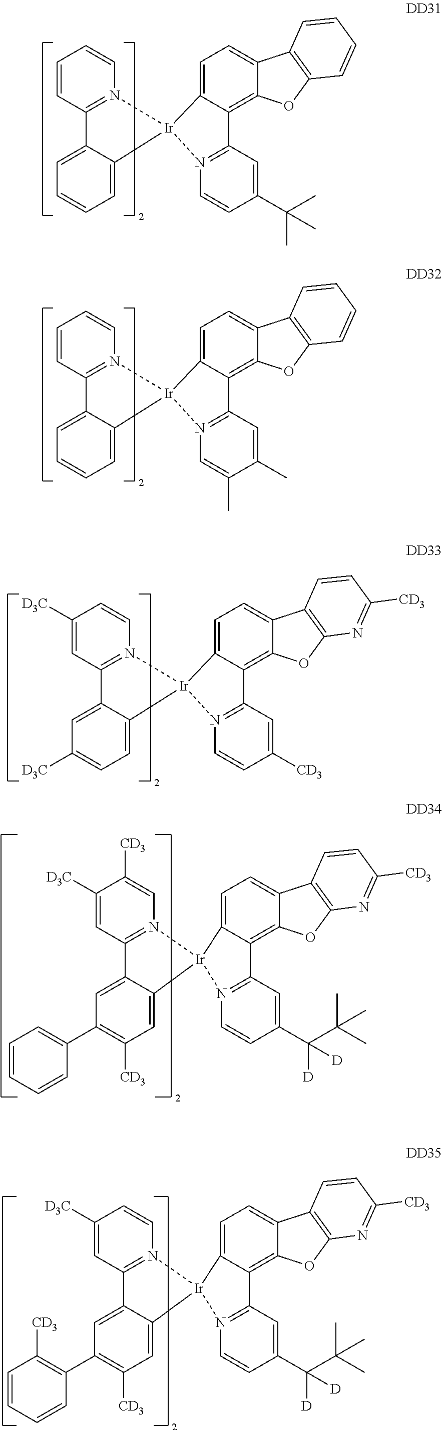

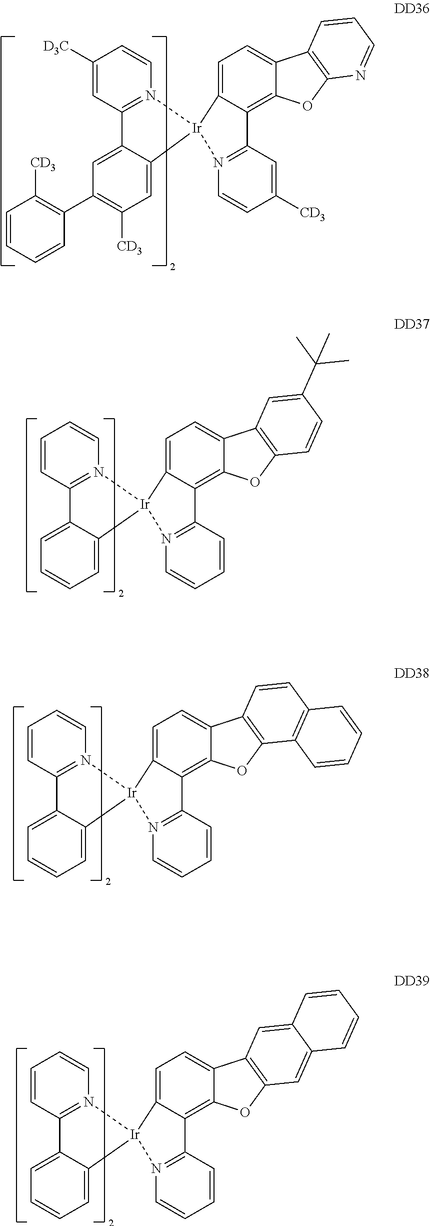

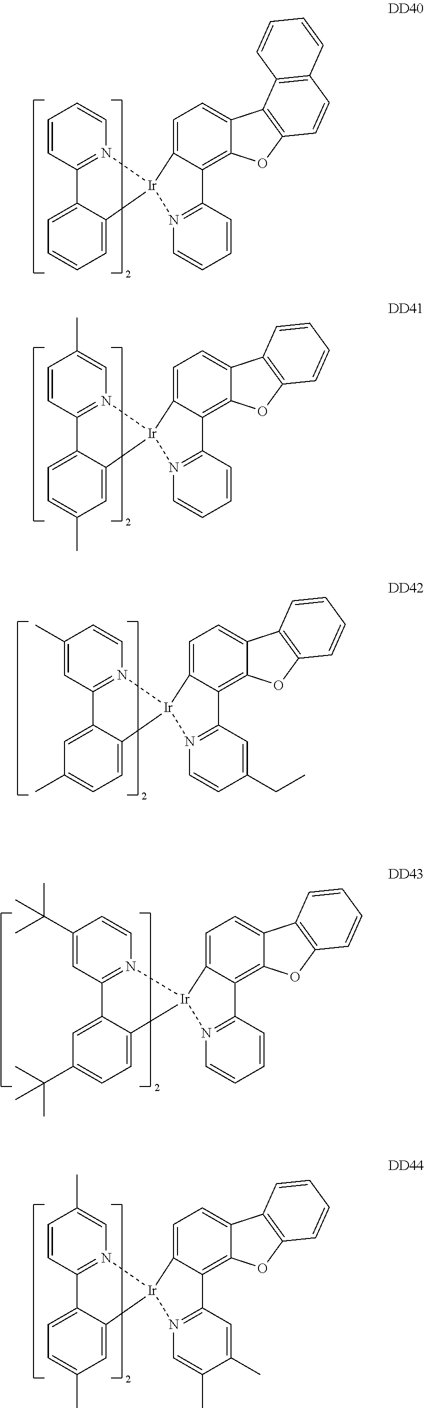

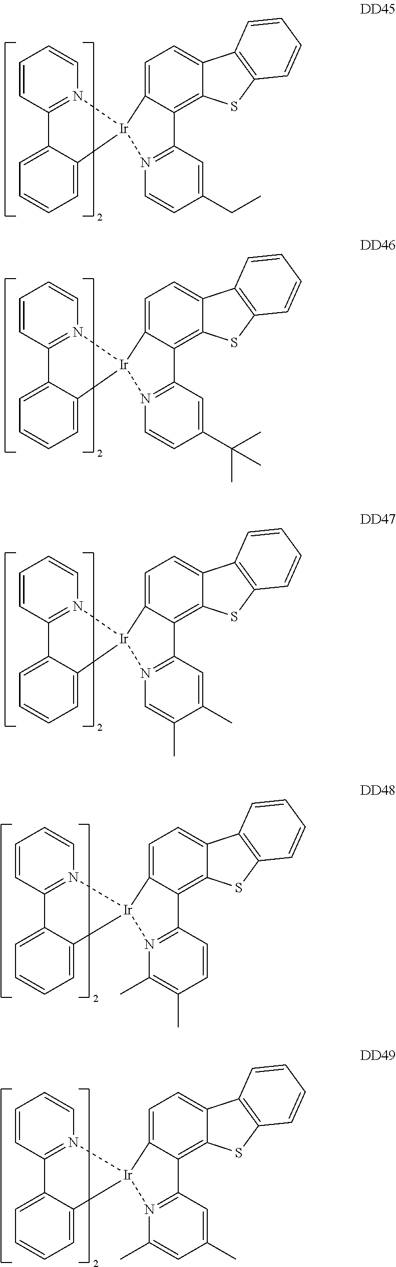

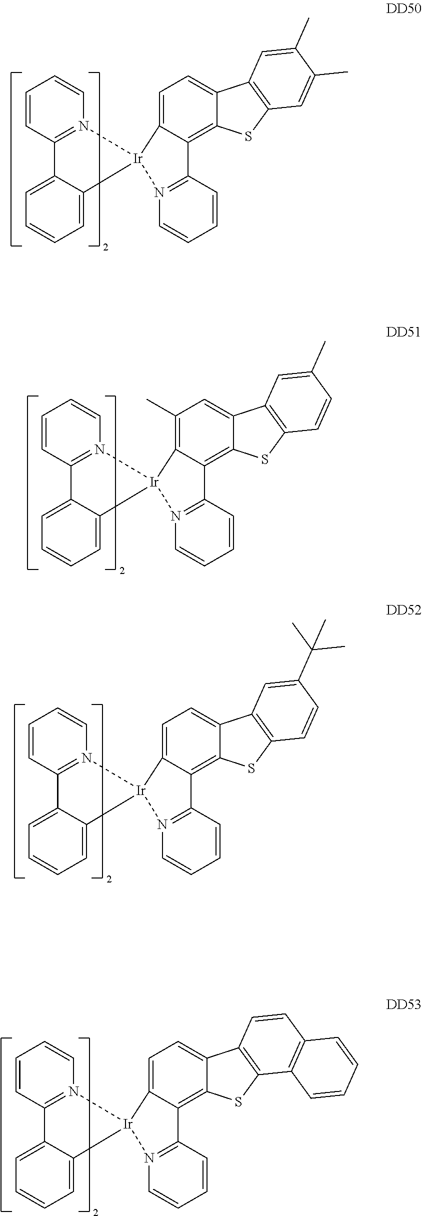

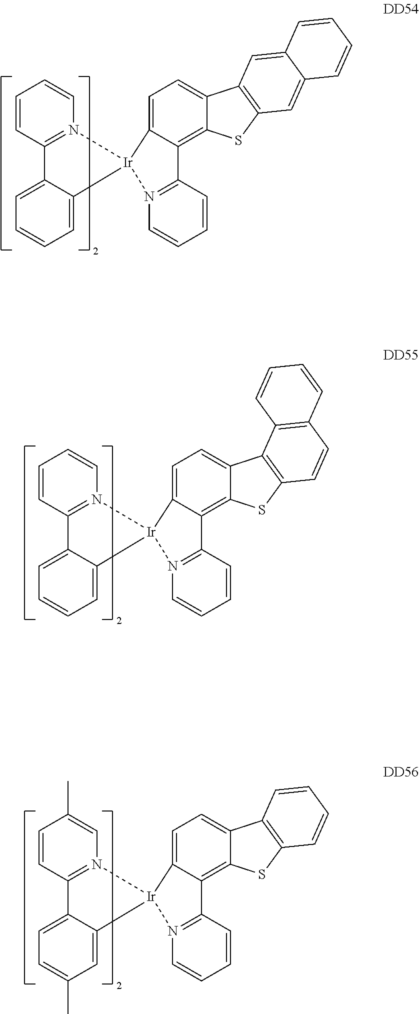

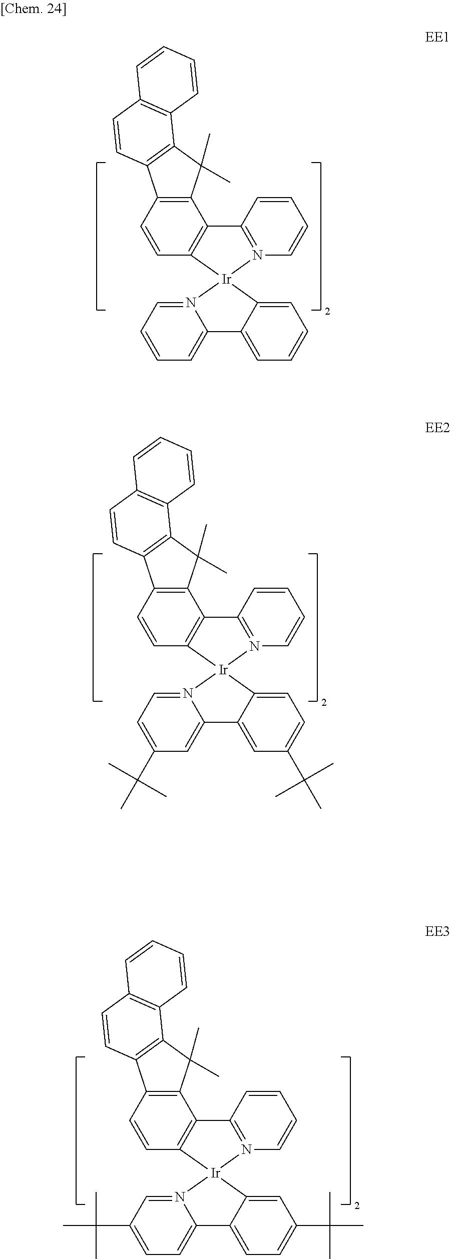

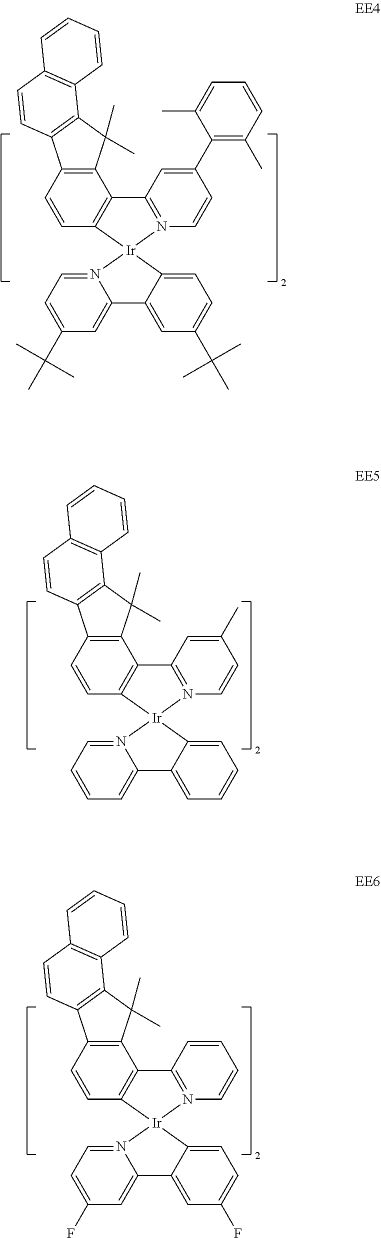

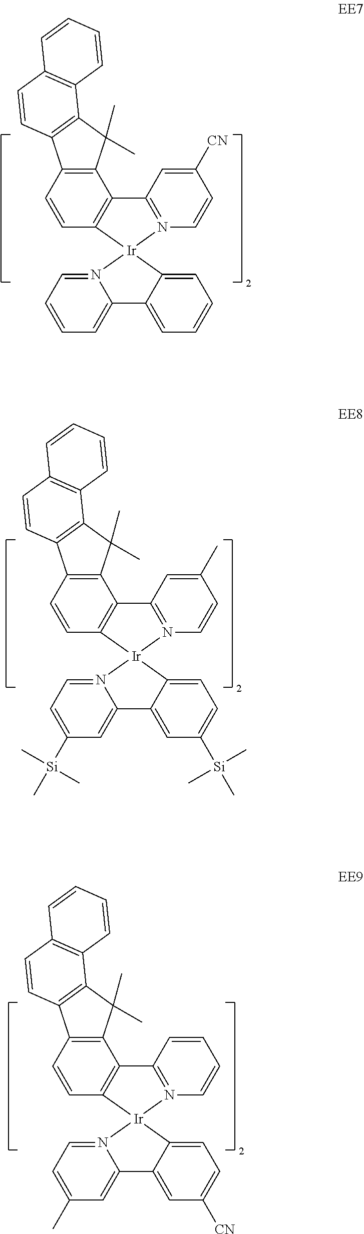

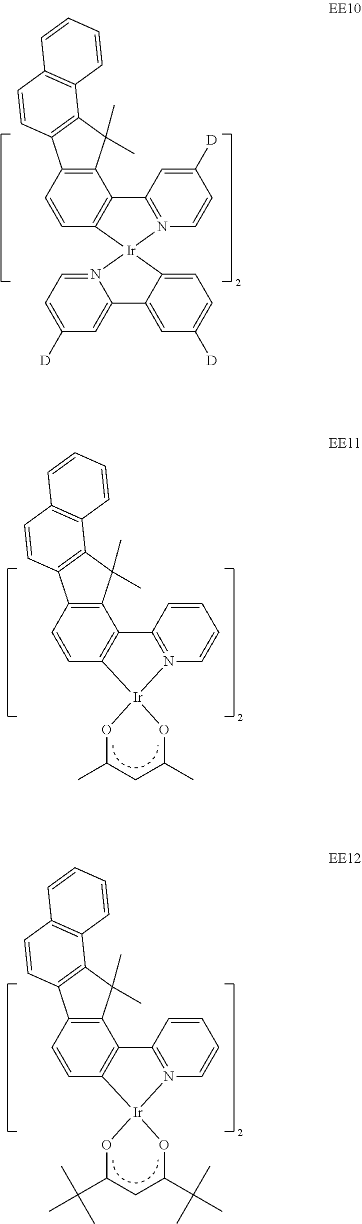

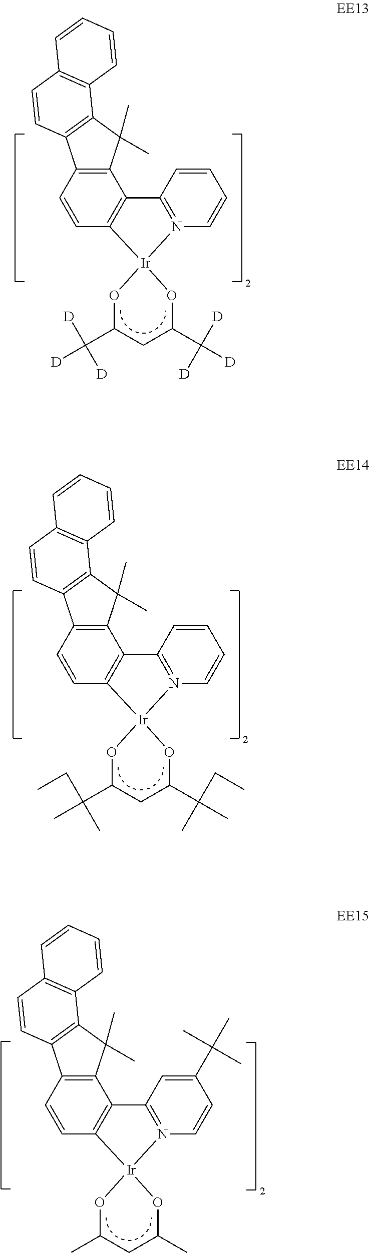

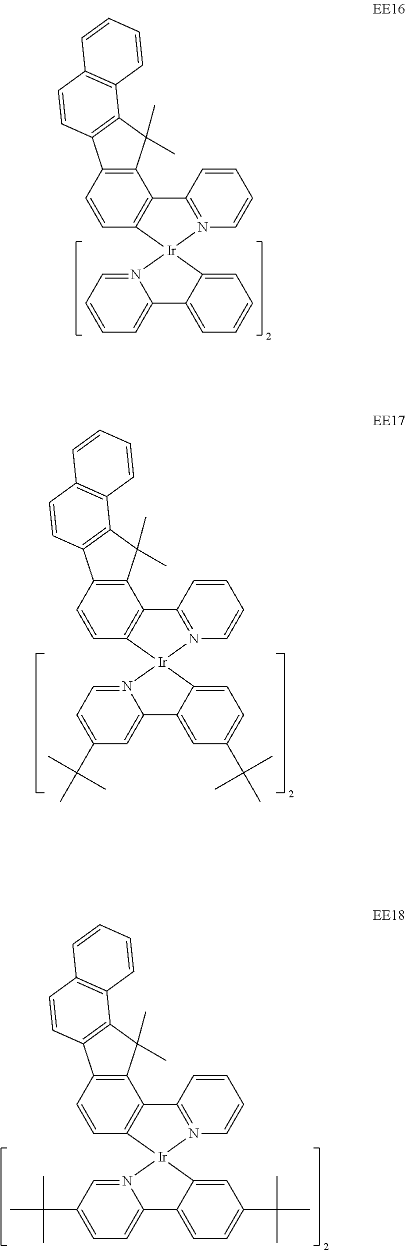

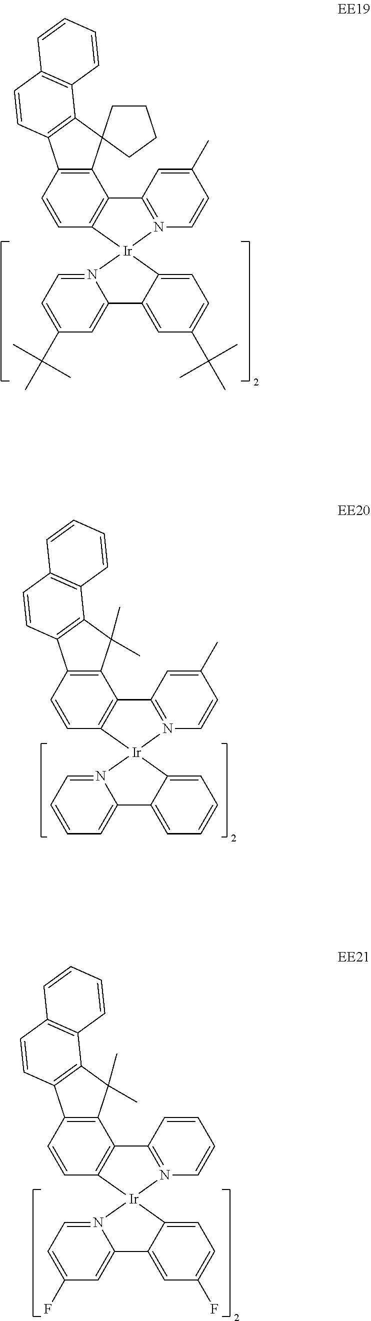

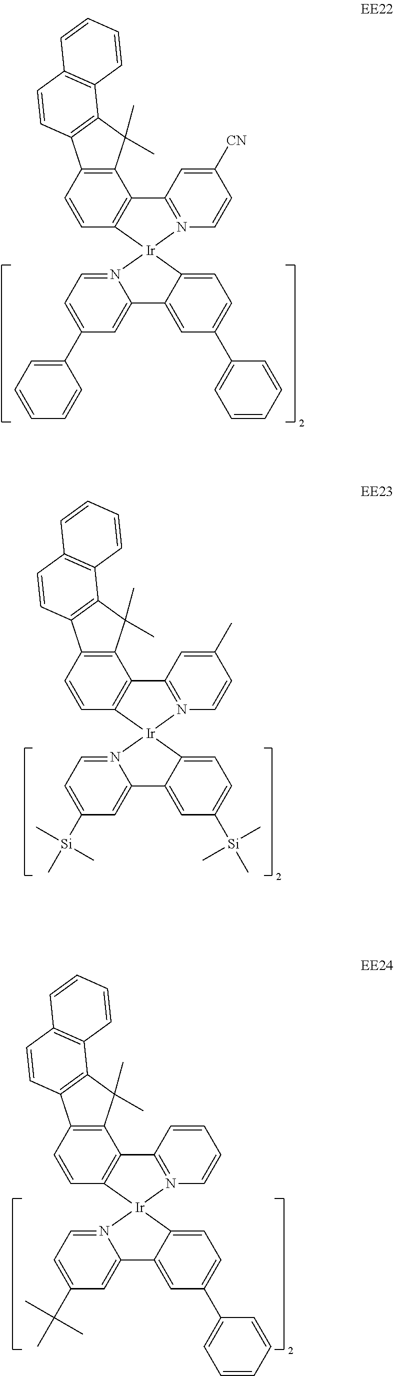

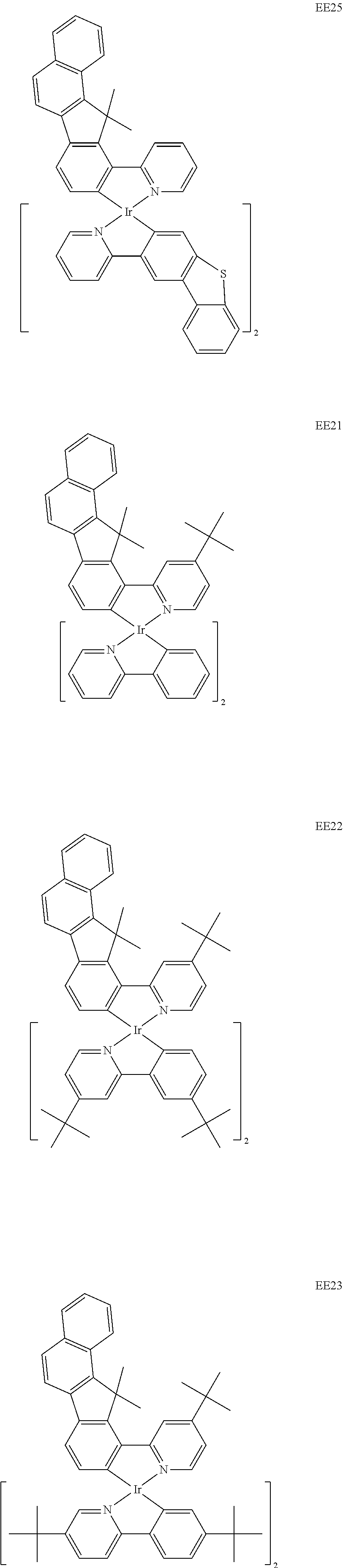

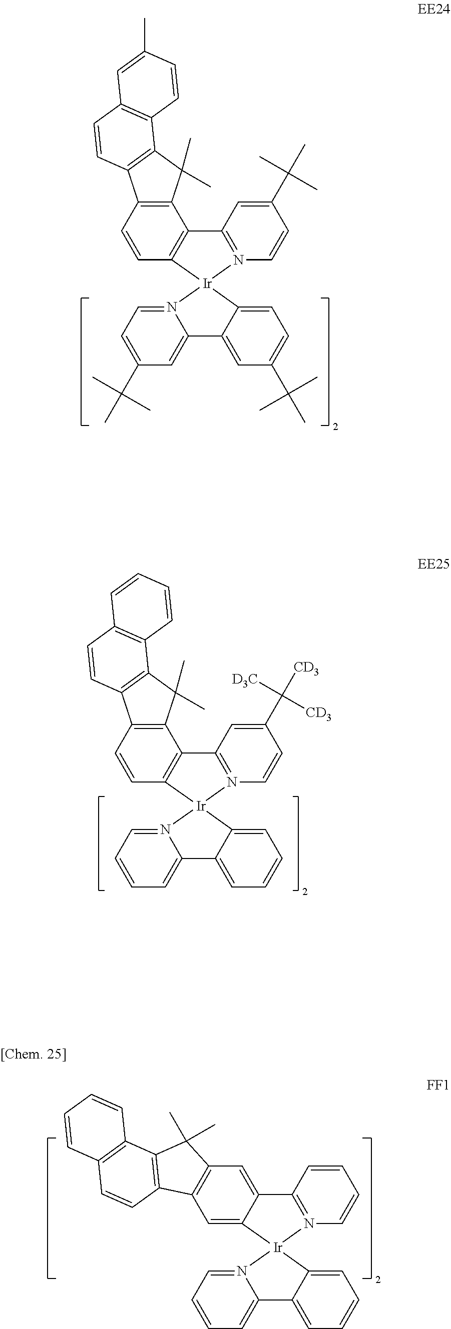

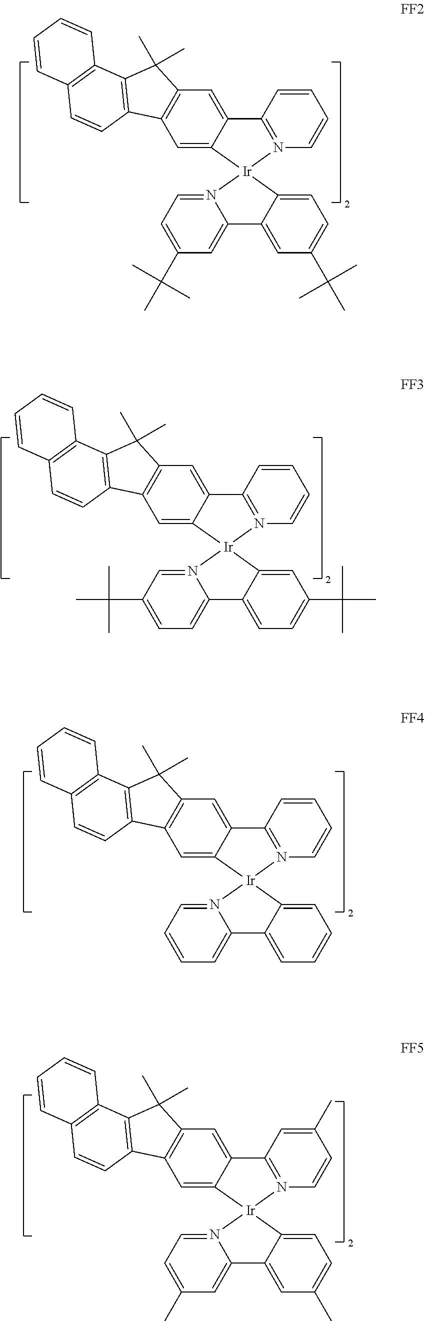

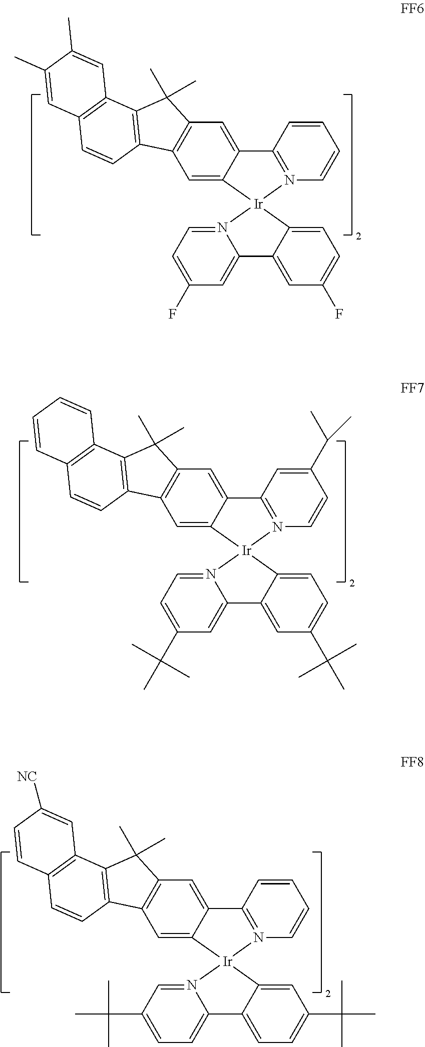

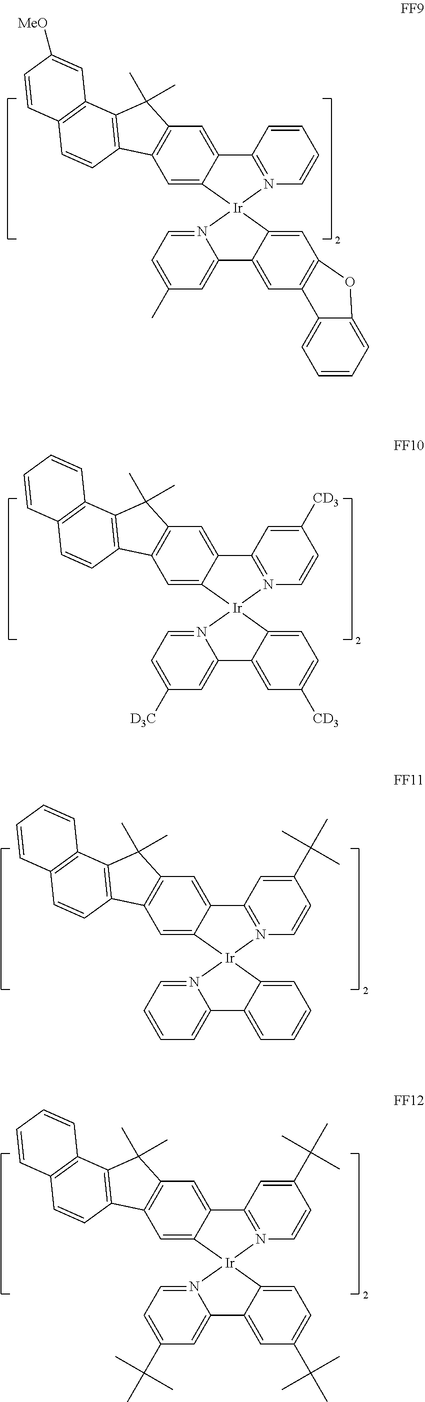

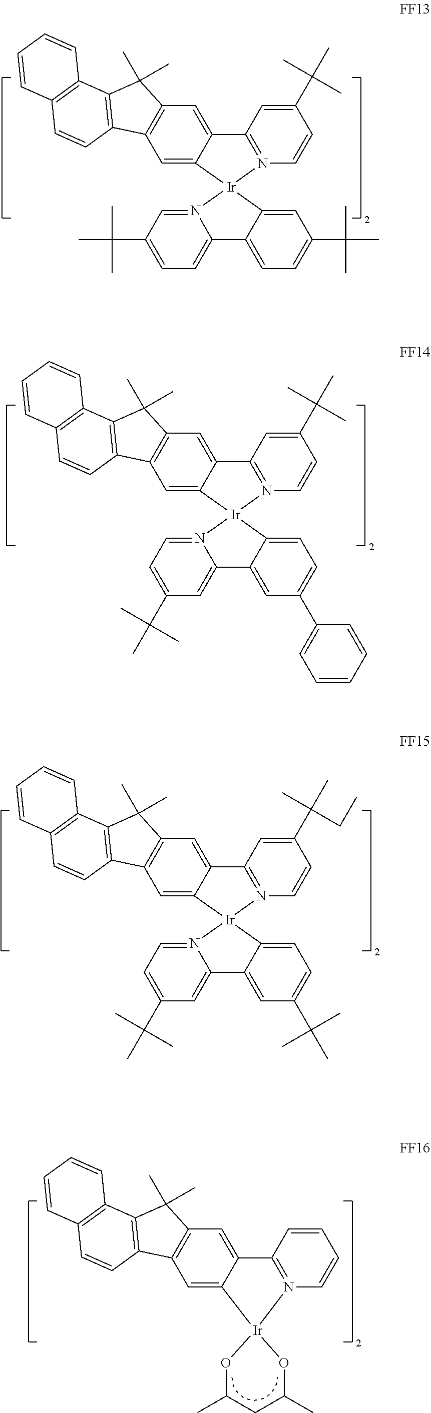

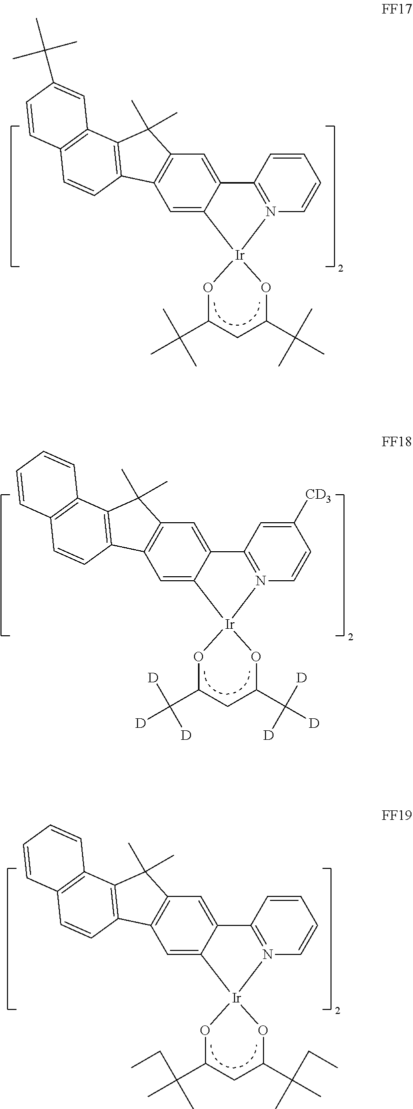

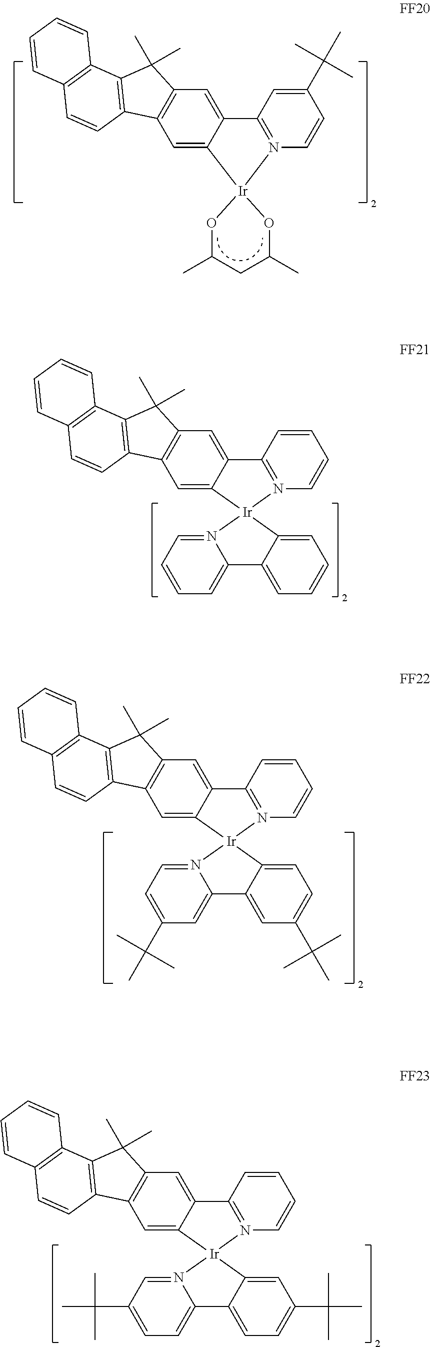

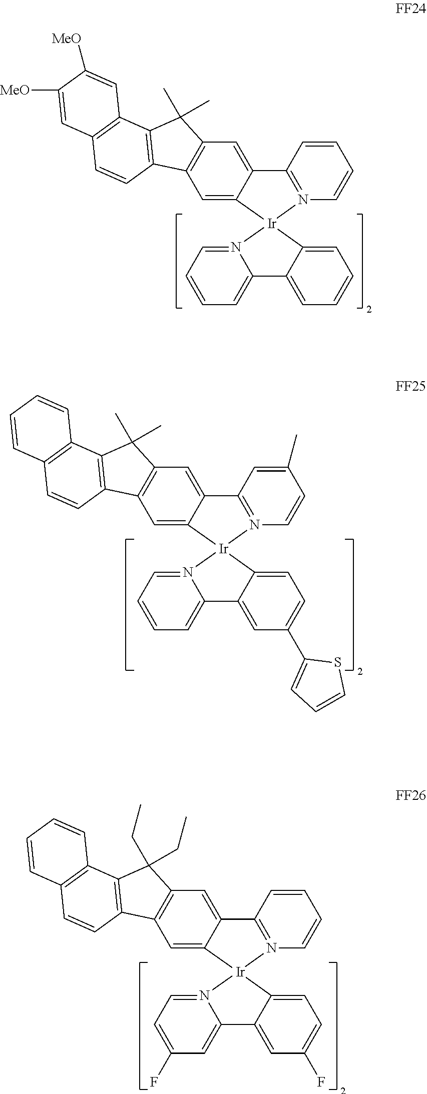

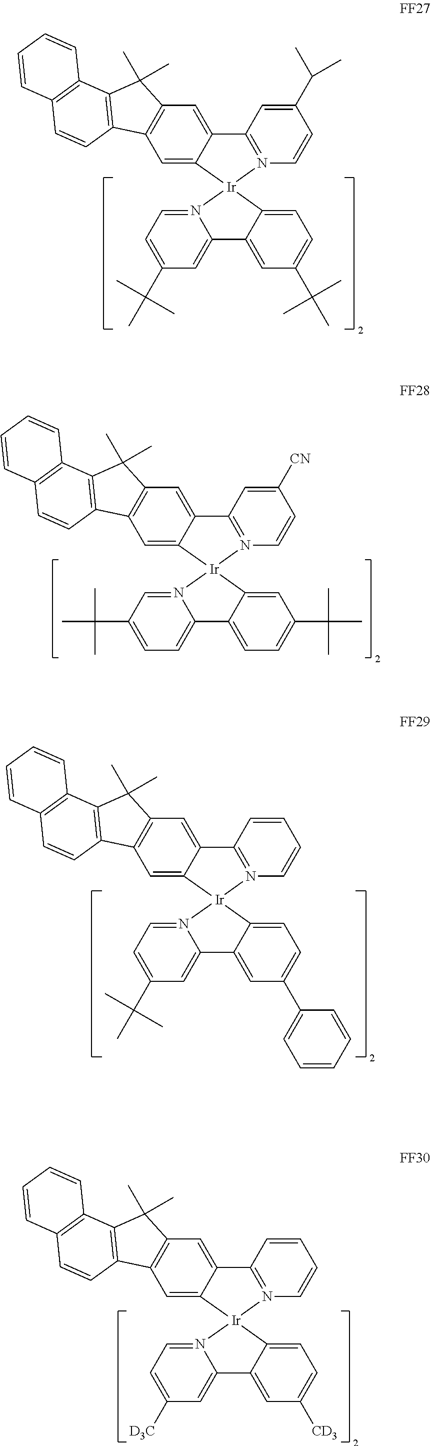

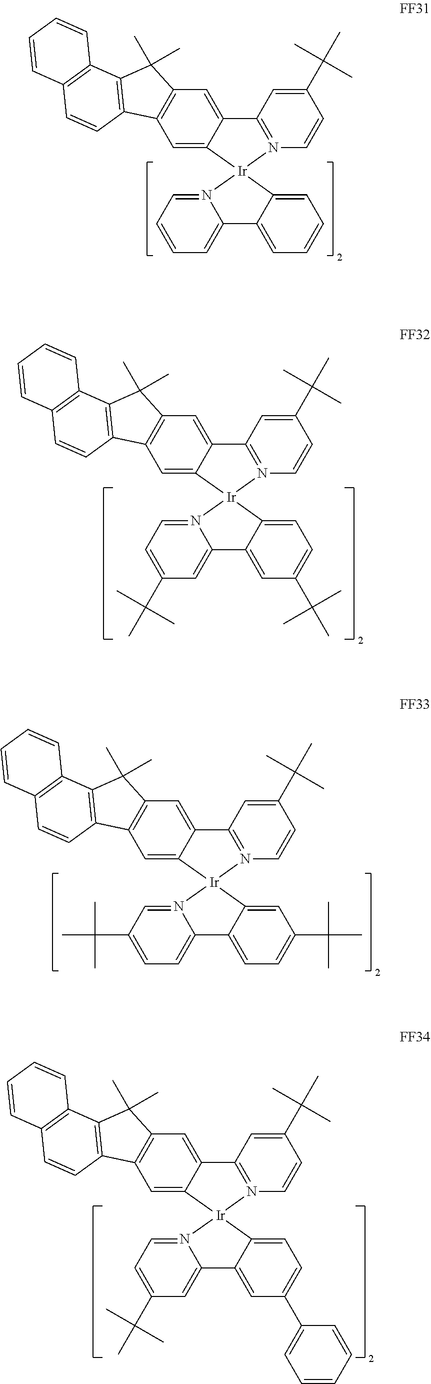

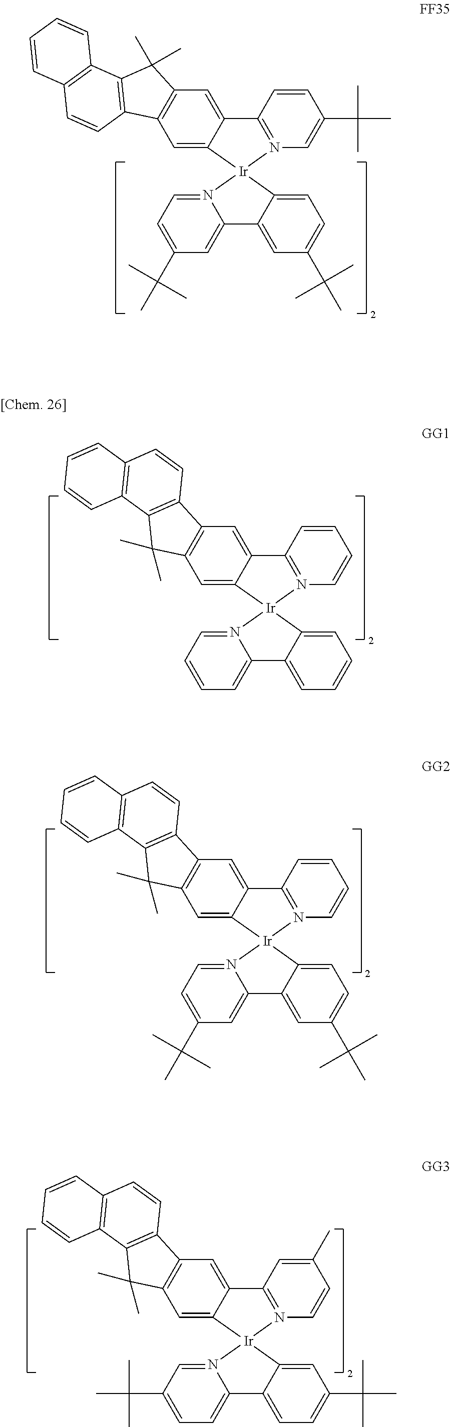

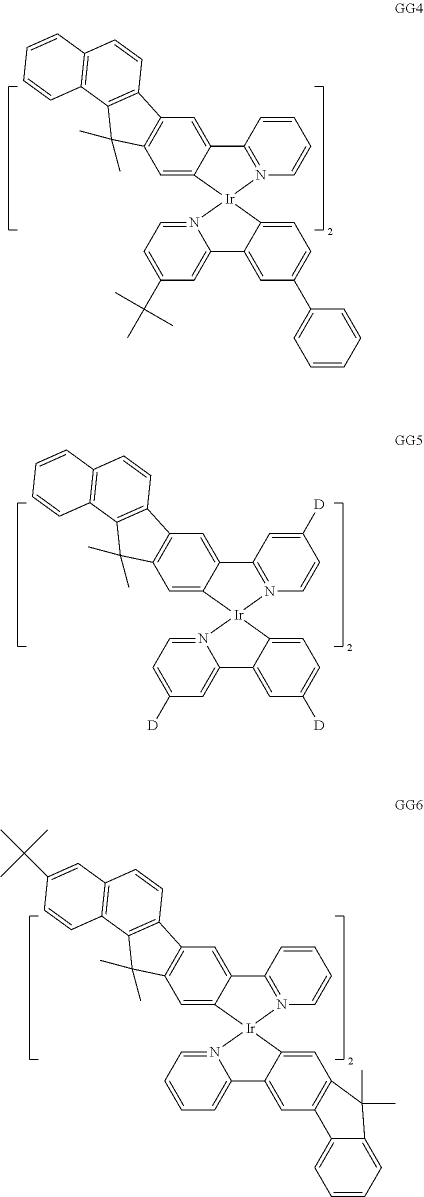

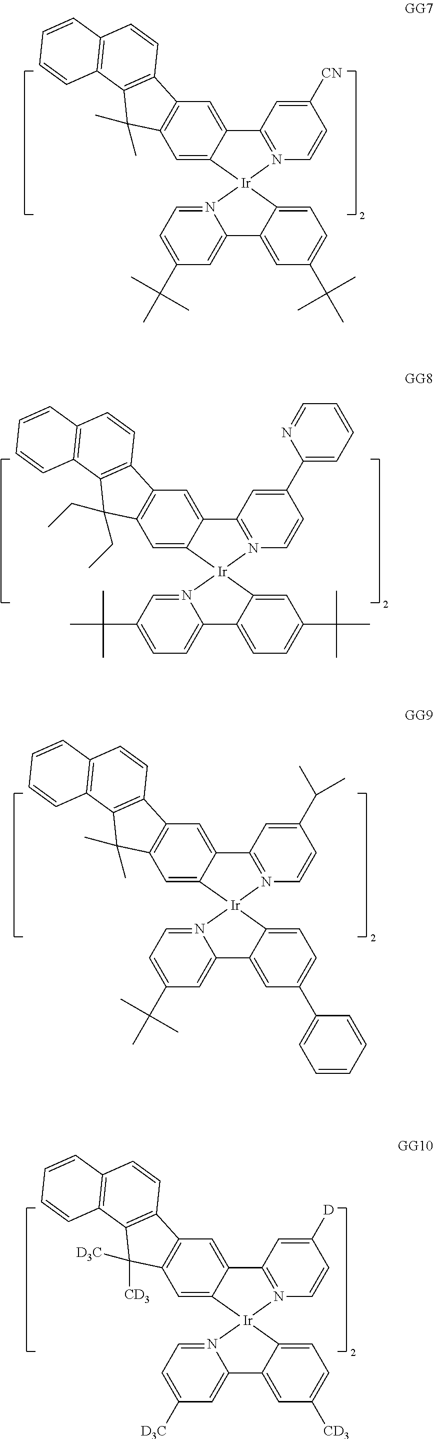

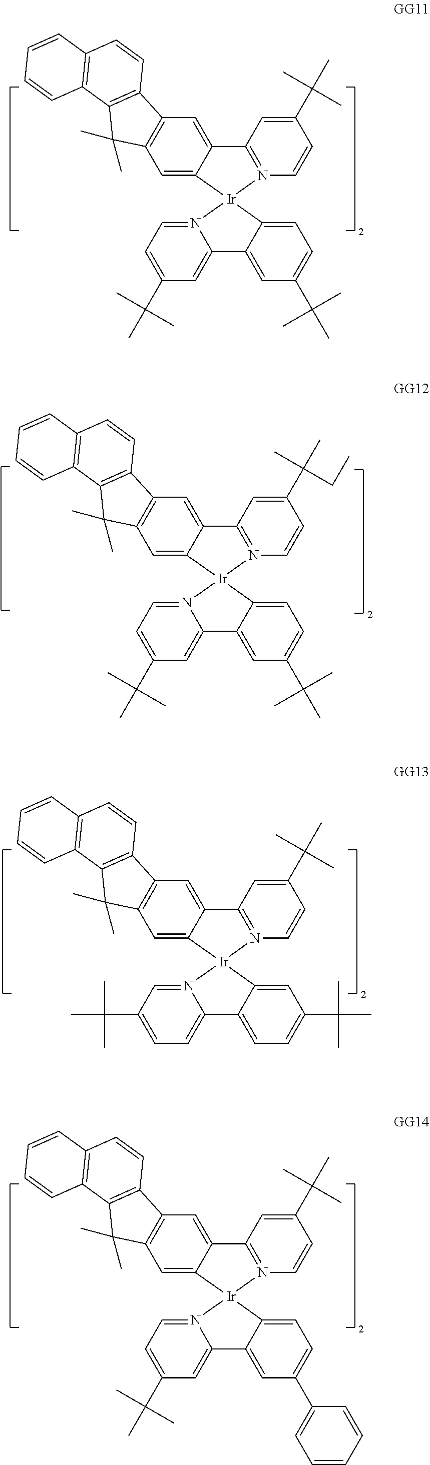

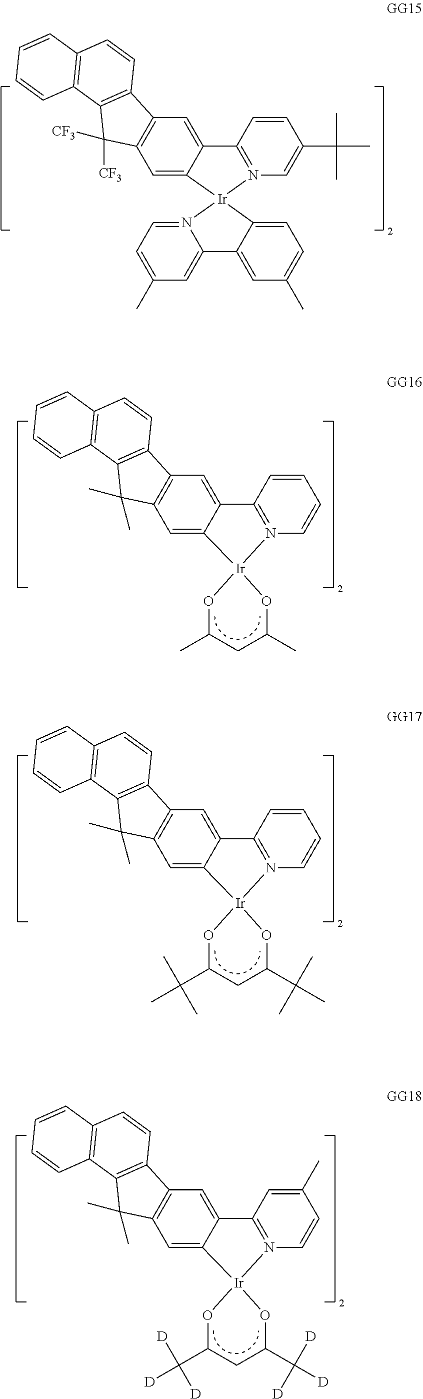

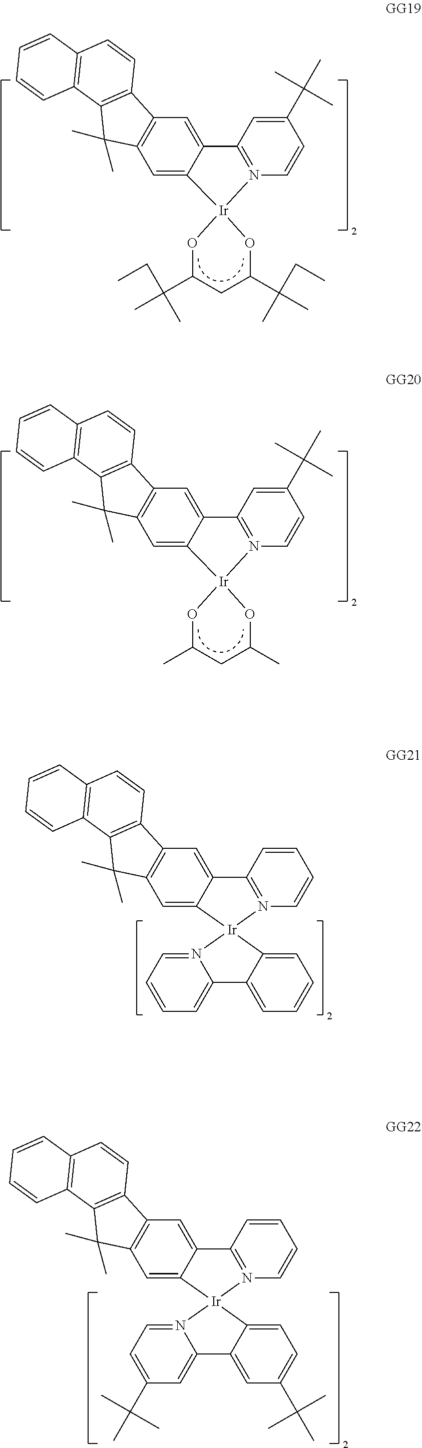

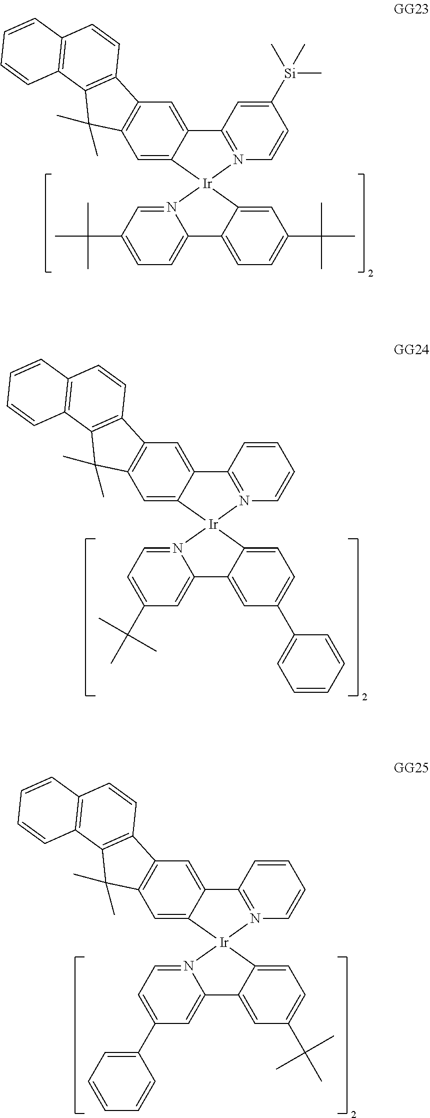

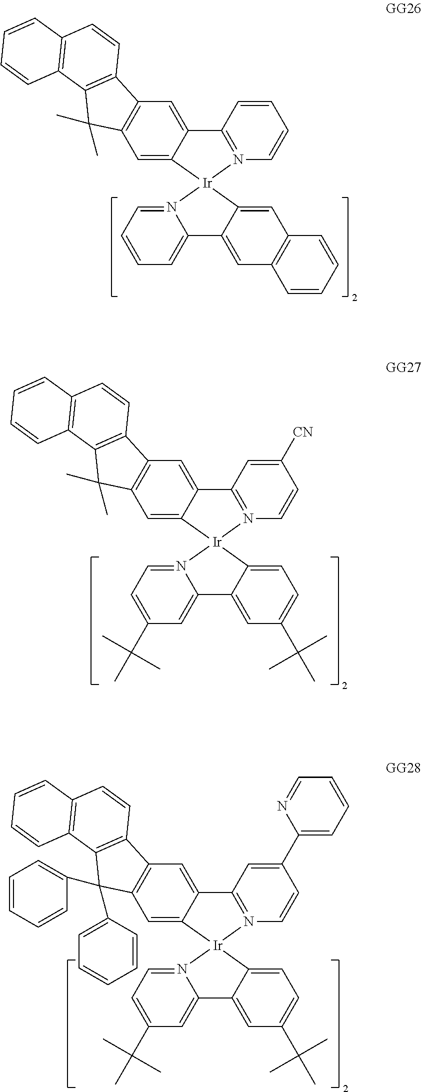

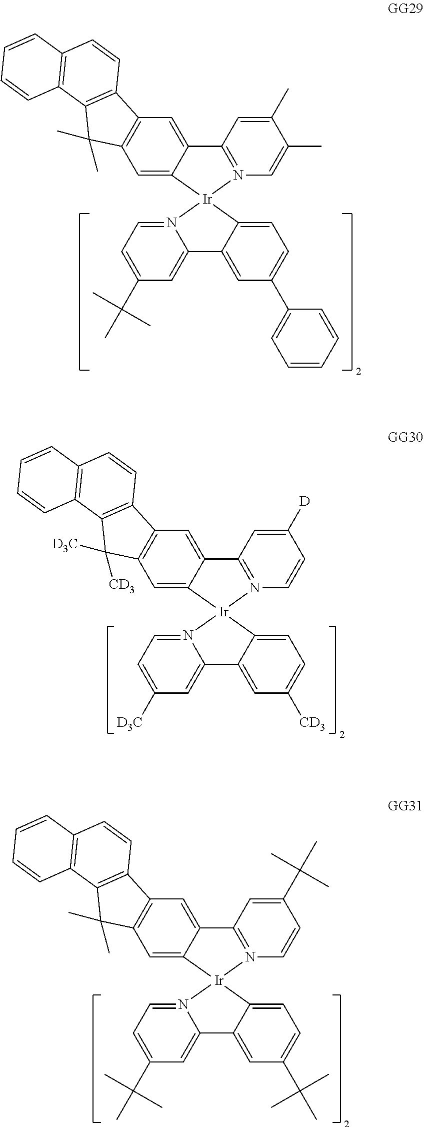

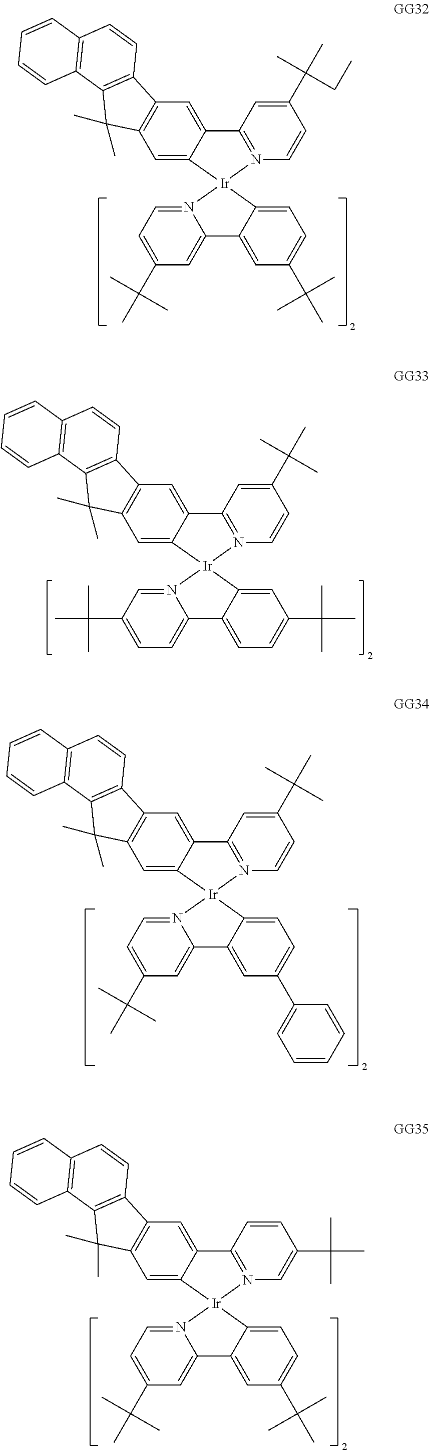

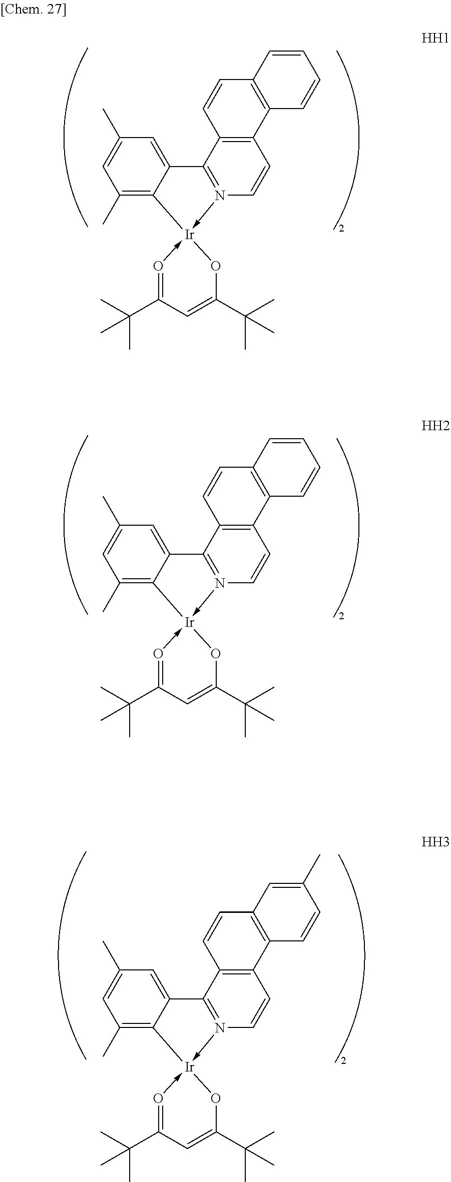

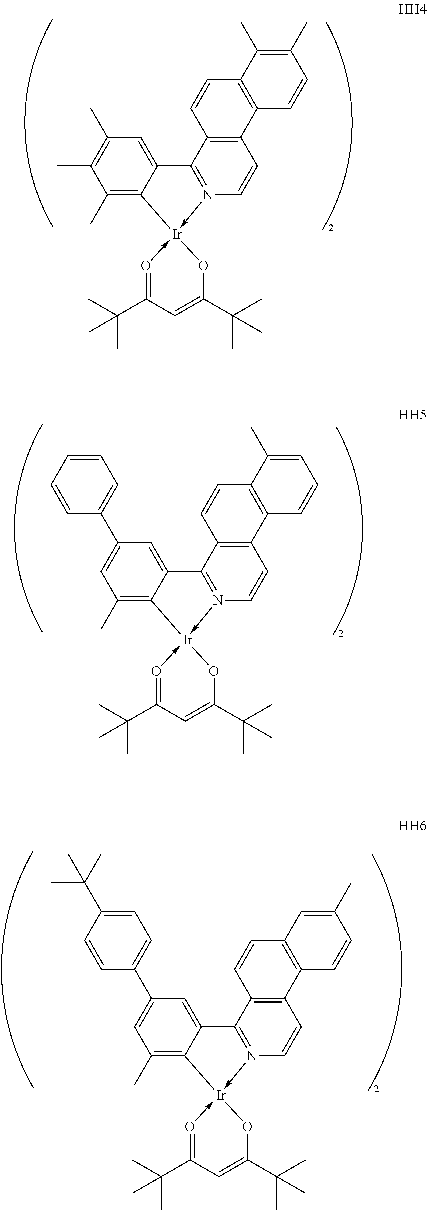

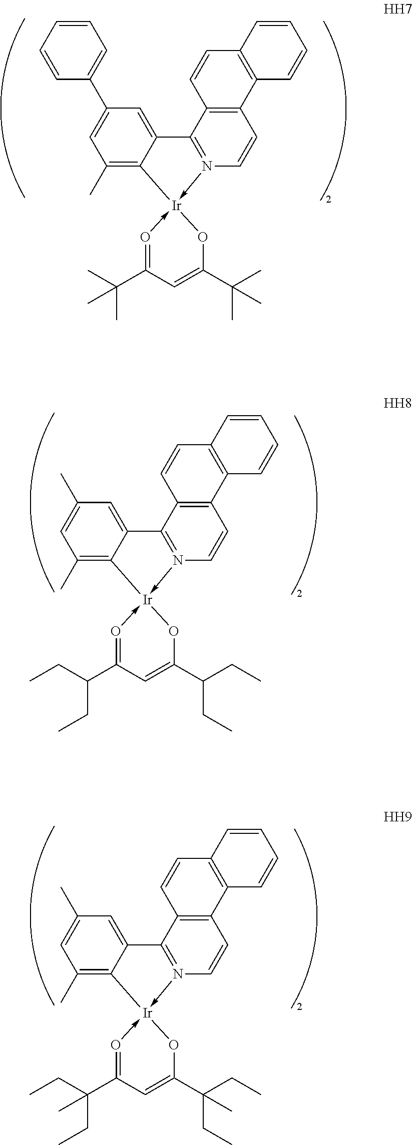

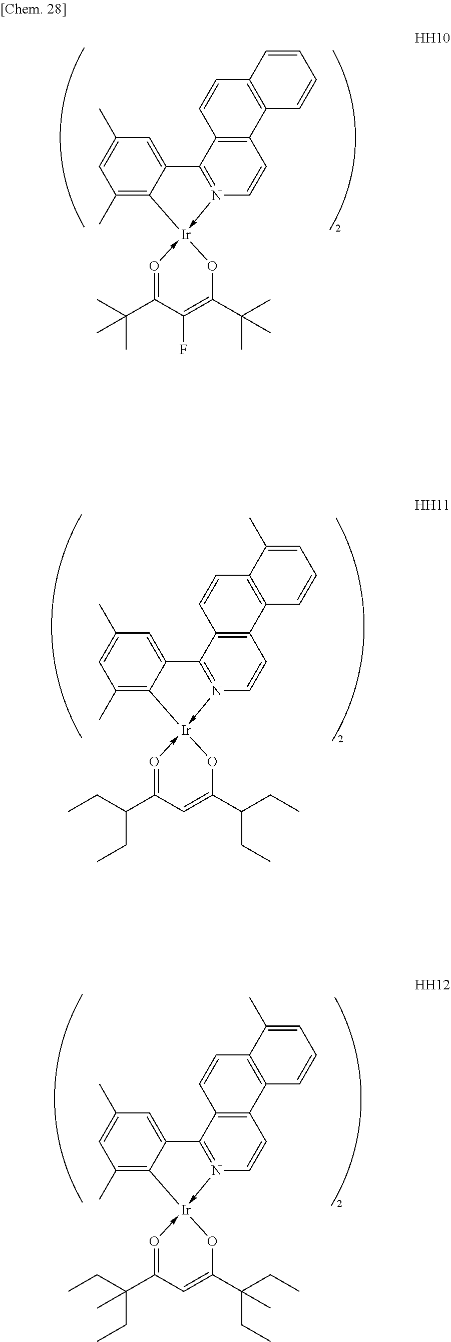

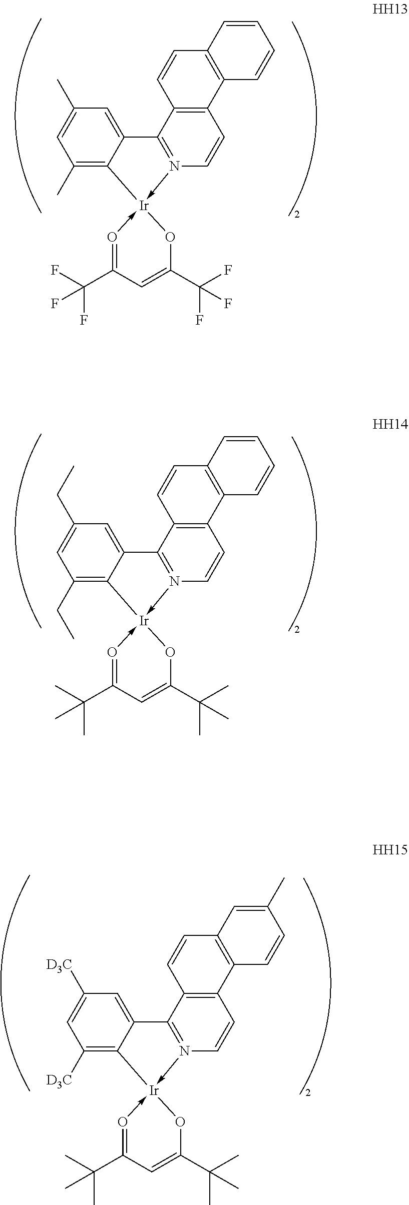

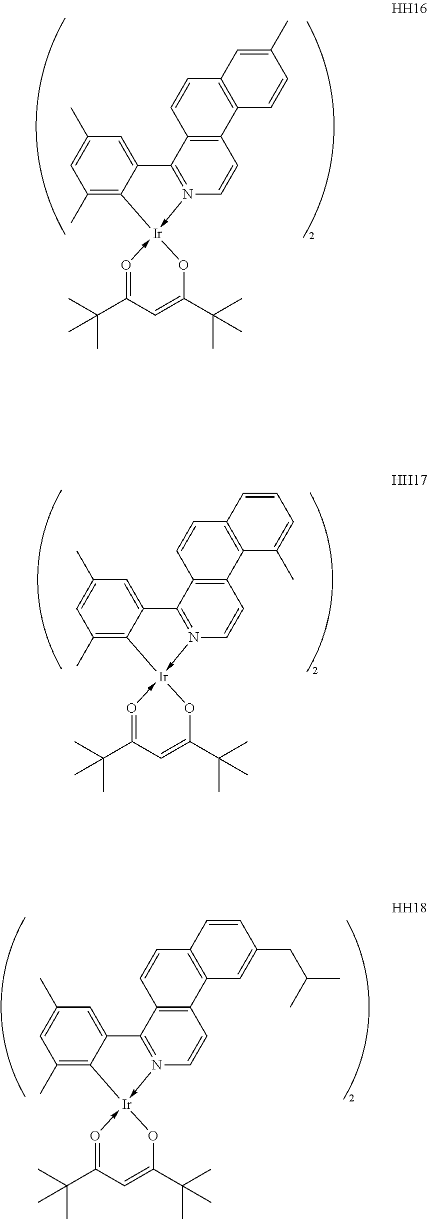

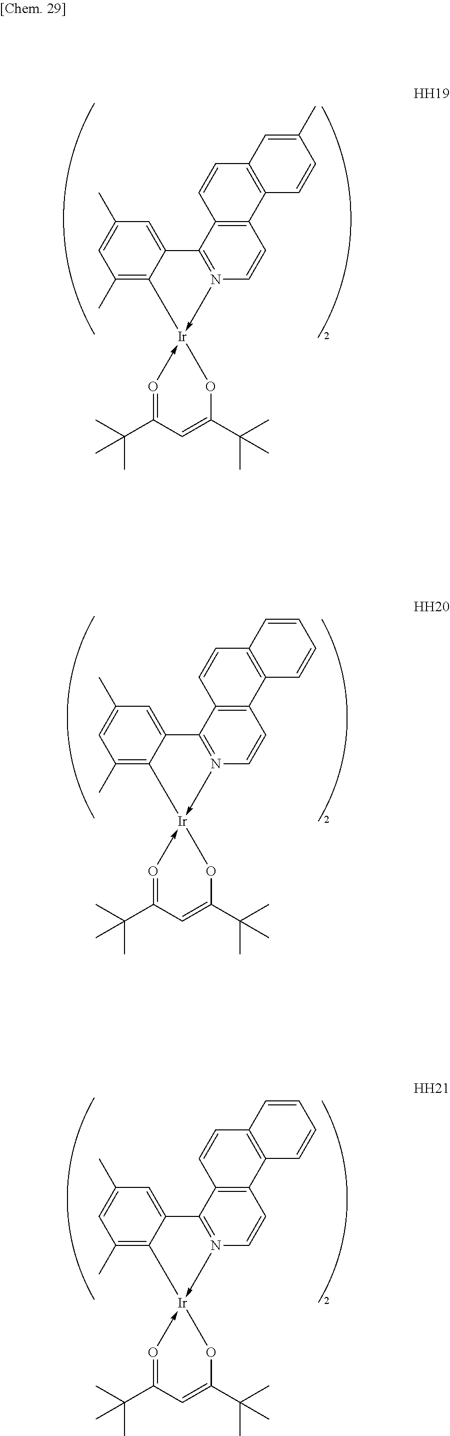

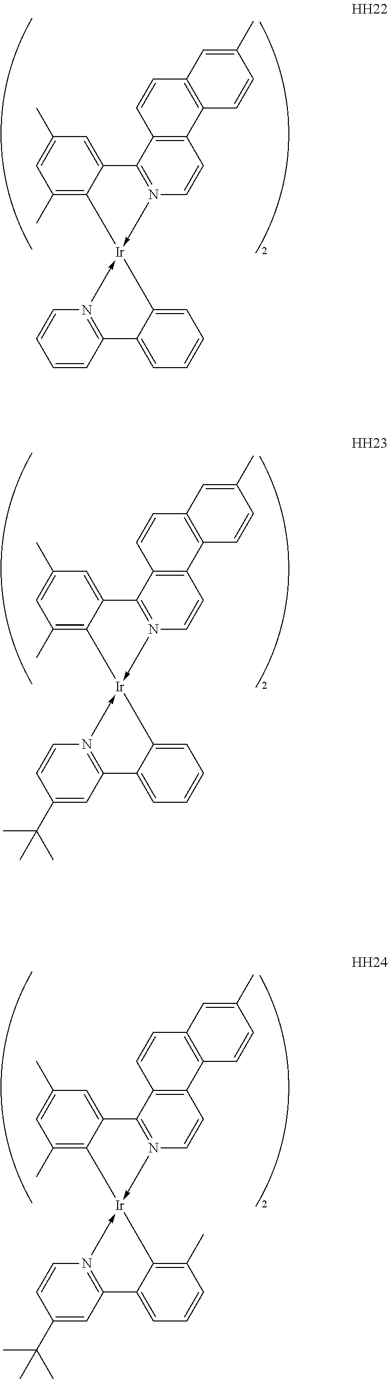

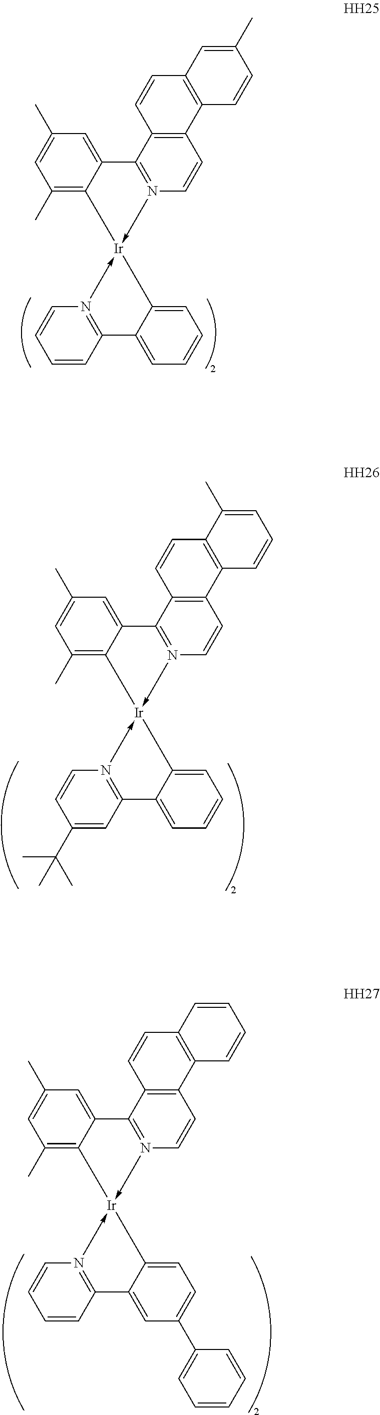

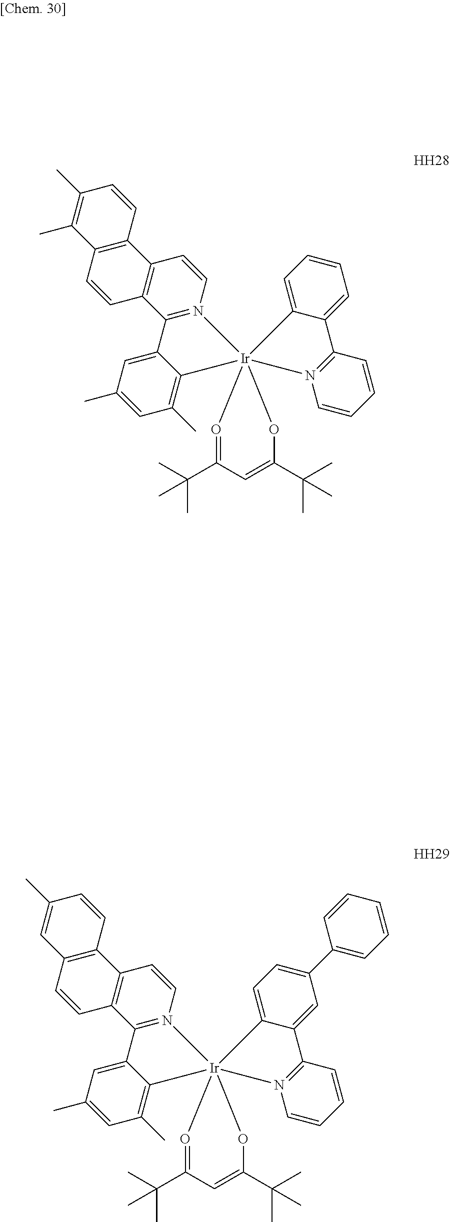

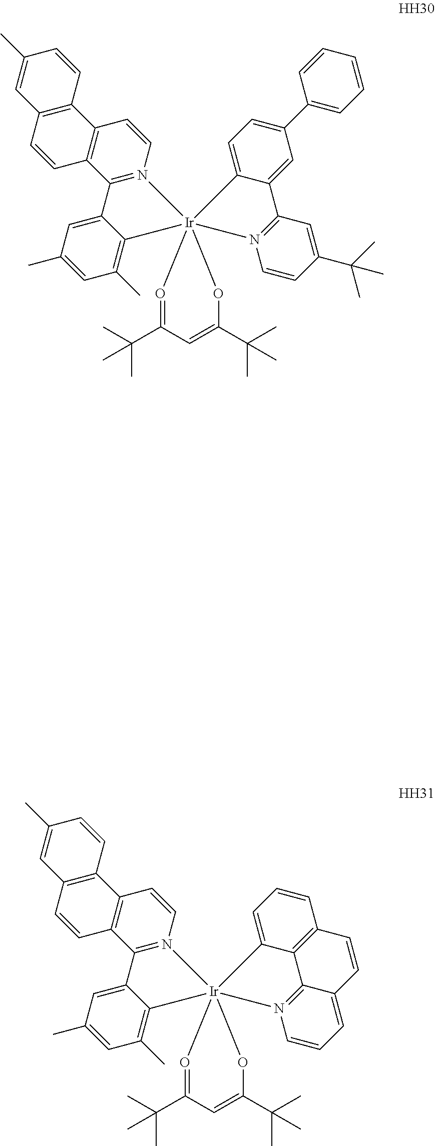

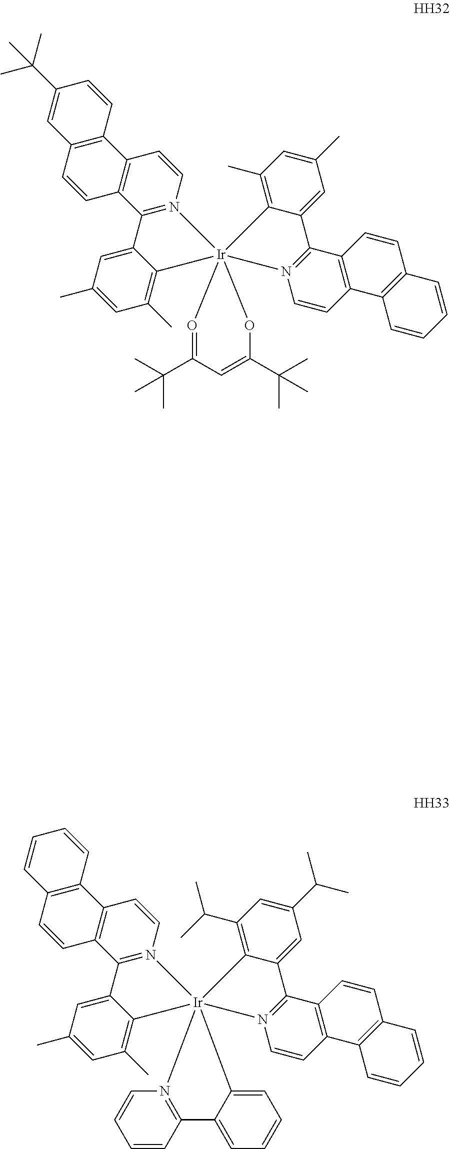

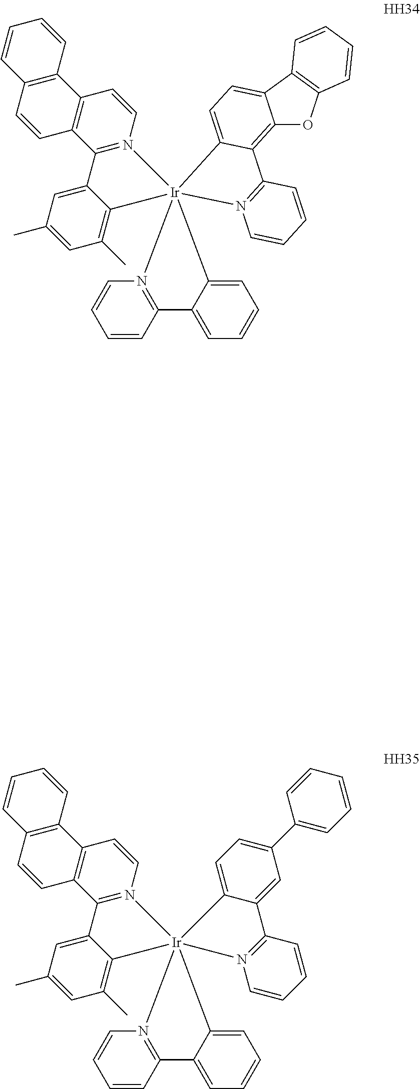

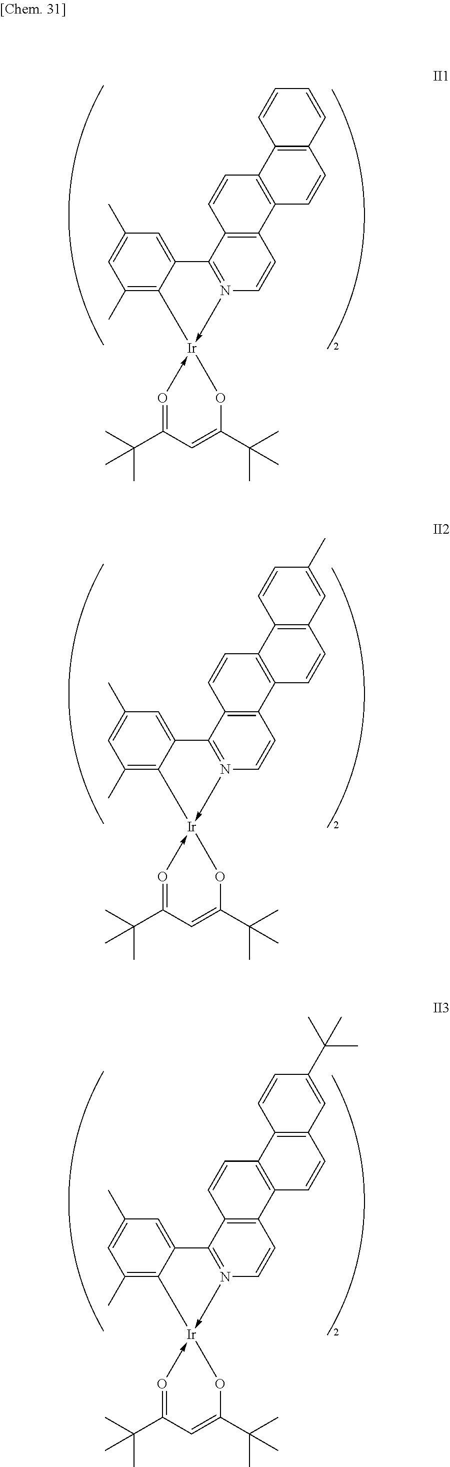

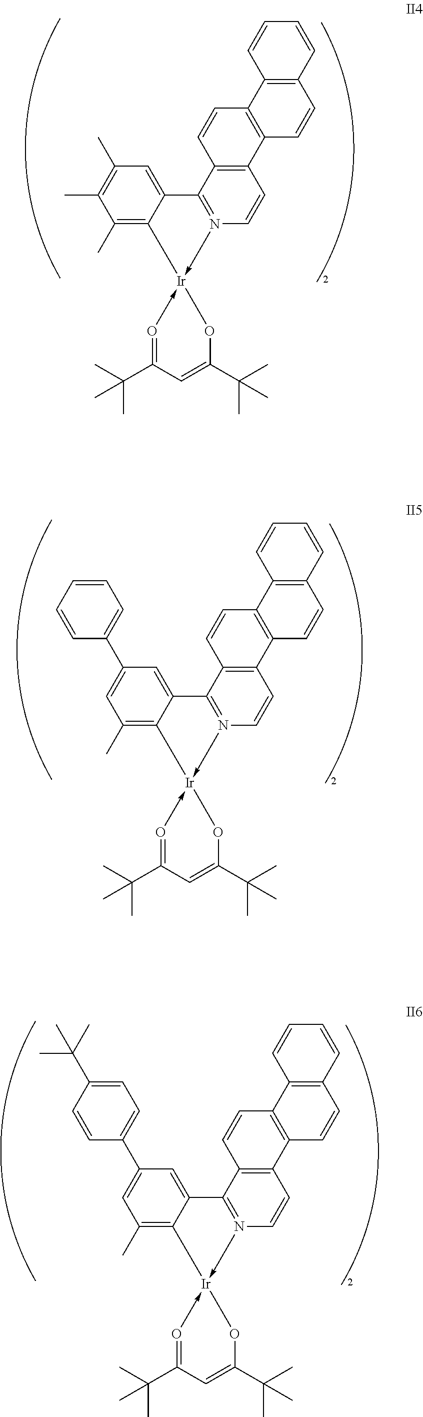

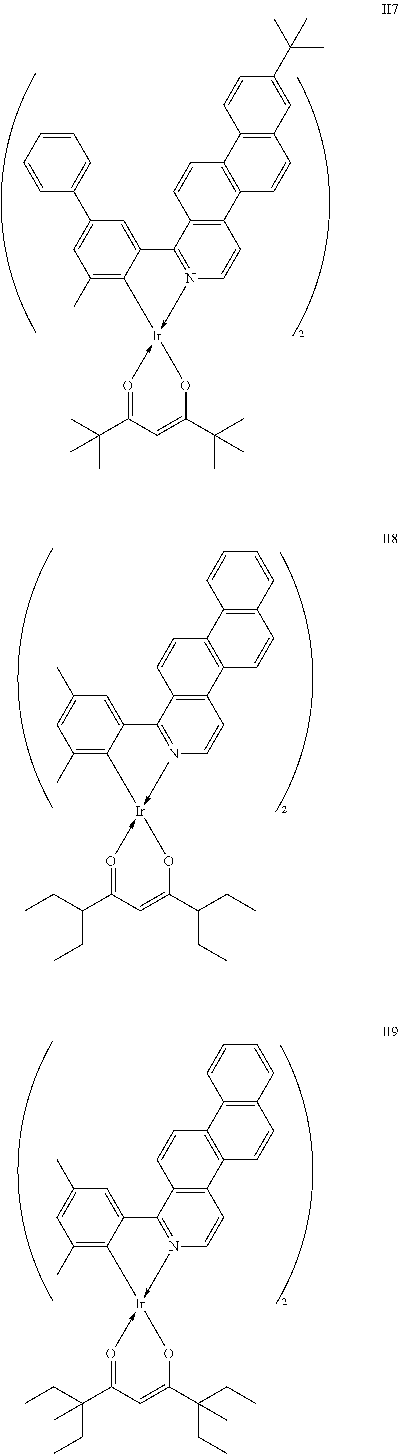

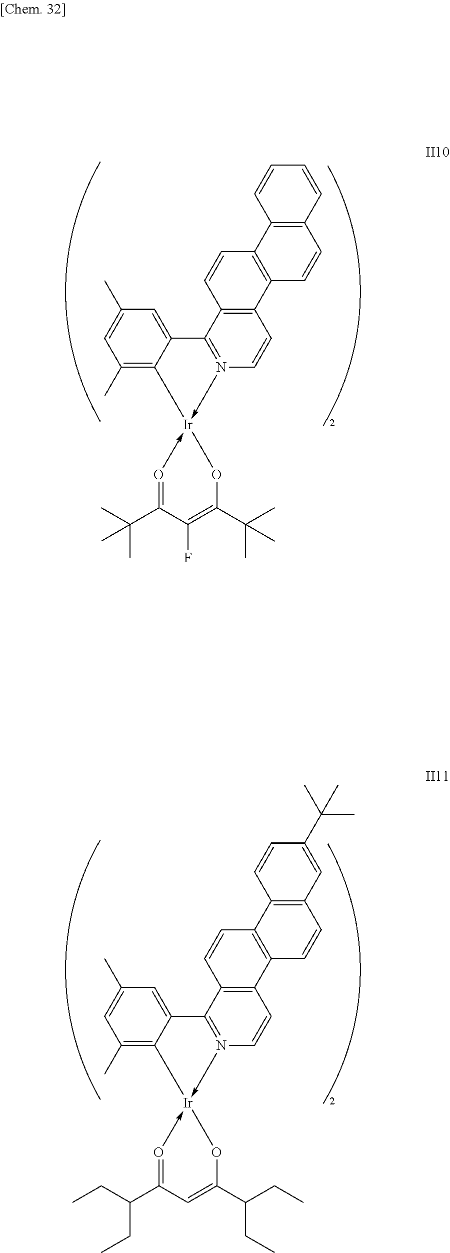

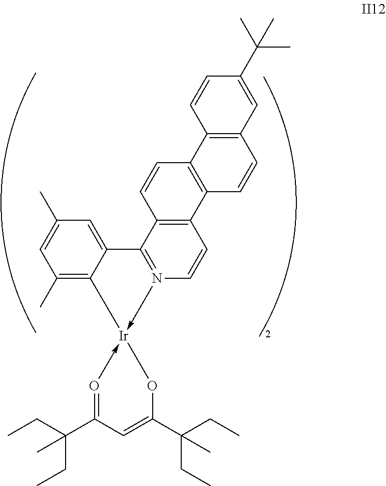

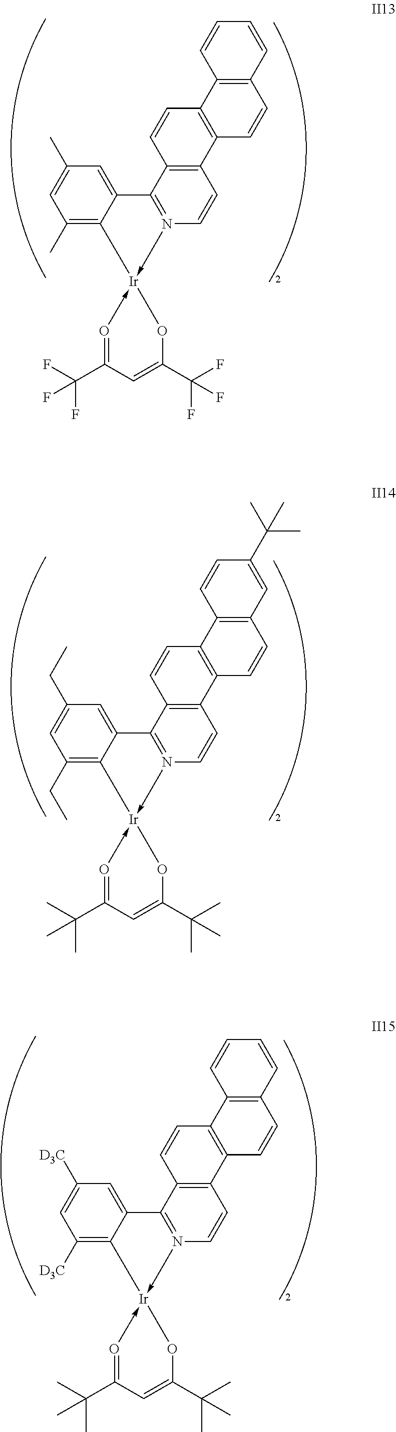

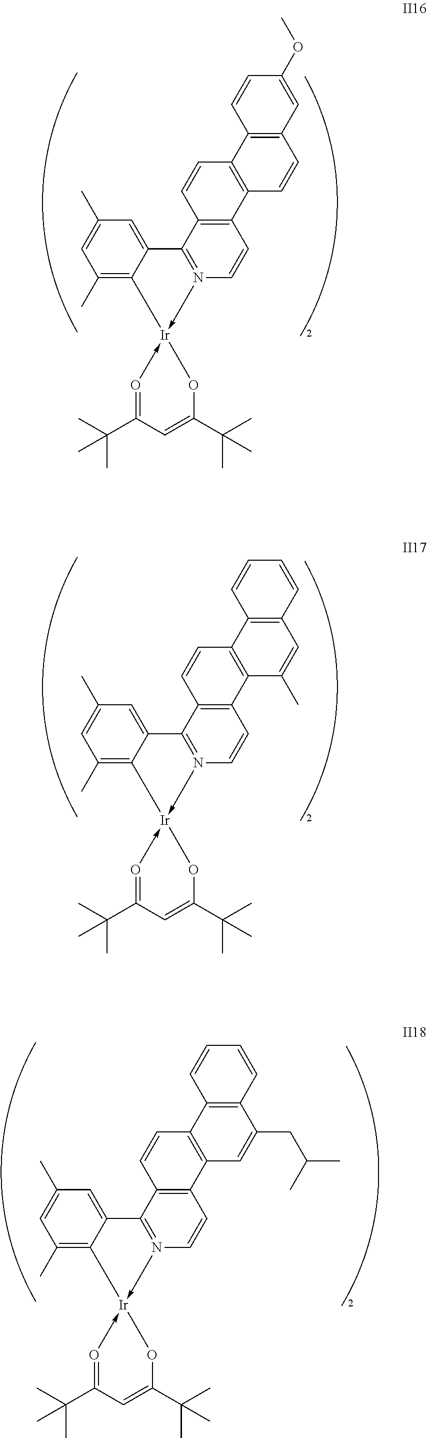

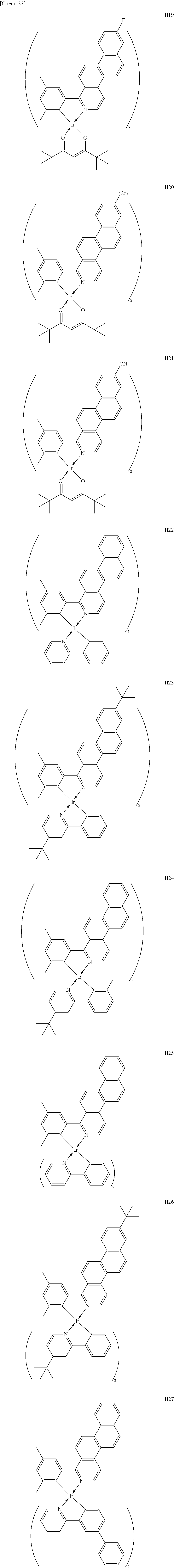

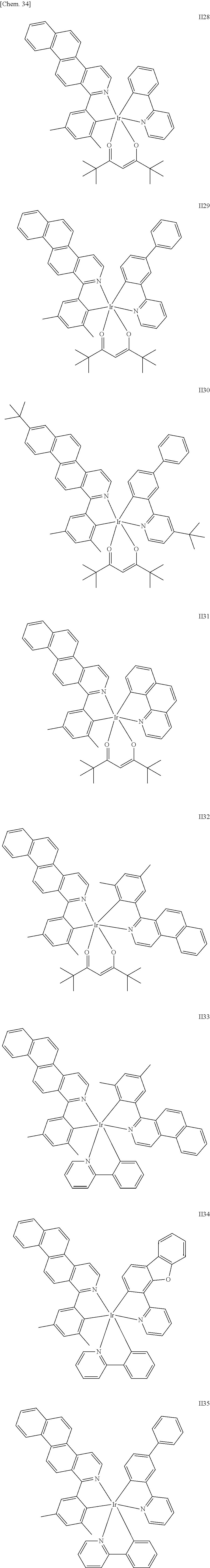

Among these organometallic complexes, the exemplary compounds belonging to the AA group and the BB group are compounds with at least a phenanthrene skeleton in a ligand of the Ir complex. Thus, the compounds have particularly high stability.

Among these organometallic complexes, the exemplary compounds belonging to the CC group are compounds with at least a triphenylene skeleton in a ligand of the Ir complex. Thus, the compounds have particularly high stability.

Among these organometallic complexes, the exemplary compounds belonging to the DD group are compounds with at least a dibenzofuran skeleton or a dibenzothiophene skeleton in a ligand of the Ir complex. Thus, in these compounds, the fused ring has an oxygen atom or a sulfur atom, and a large number of lone pairs of these atoms can enhance charge transport properties. Thus, the compounds are particularly easy to adjust the carrier balance.

Among these organometallic complexes, the exemplary compounds belonging to the EE group, the FF group, and the GG group are compounds with at least a benzofluorene skeleton in a ligand of the Ir complex. These compounds further have a substituent at the 9-position of the fluorene. Thus, the substituent in the direction perpendicular to the in-plane direction of the fluorene ring can particularly suppress overlapping of fused rings. Thus, the compounds have particularly high sublimability.

Among these organometallic complexes, the exemplary compounds belonging to the HH group are compounds with at least a benzoisoquinoline skeleton in a ligand of the Ir complex. These compounds contain a nitrogen atom in the fused ring, and a lone pair and high electronegativity of the nitrogen atom can enhance charge transport properties. Thus, the compounds are particularly easy to adjust the carrier balance.

Among these organometallic complexes, the exemplary compounds belonging to the II group are compounds with at least a naphthoisoquinoline skeleton in a ligand of the Ir complex. These compounds contain a nitrogen atom in the fused ring, and a lone pair and high electronegativity of the nitrogen atom can enhance charge transport properties. Thus, the compounds are particularly easy to adjust the carrier balance.

(2-3) When the second compound has a structure of at least one of a carbazole skeleton, an azine ring, and a xanthone skeleton, the light emission efficiency is high.

An organic compound according to the present invention has a large band gap when Ar₁ and Ar₂ have a tricyclic or polycyclic fused-ring structure. Thus, when used for a light-emitting layer, an organic compound according to the present invention may increase the barrier to carrier injection from a peripheral layer.

Thus, the assist material preferably has any one of a carbazole skeleton, an azine ring, and a xanthone skeleton. This is because these materials have high electron-donating ability and electron-withdrawing ability, so that HOMO and LUMO can be easily adjusted and injection of a carrier from a peripheral layer can be promoted.

Such an assist material in combination with an organic compound according to the present invention can achieve a good carrier balance. Thus, by using such an assist material, an organic compound according to the present invention can provide an organic light-emitting device with higher light emission efficiency.

### (3) Other Compounds

Examples of other compounds that can be used for the organic light-emitting device according to the present embodiment are described below.

A hole injection/transport material suitably used for a hole injection layer or a hole transport layer preferably has high hole mobility to facilitate the injection of a hole from a positive electrode and to transport the injected hole to a light-emitting layer. Furthermore, a material with a high glass transition temperature is preferred to reduce degradation of film quality, such as crystallization, in an organic light-emitting device. Examples of a low-molecular-weight or high-molecular-weight material with hole injection/transport ability include, but are not limited to, a triarylamine derivative, an aryl carbazole derivative, a phenylenediamine derivative, a stilbene derivative, a phthalocyanine derivative, a porphyrin derivative, polyvinylcarbazole, polythiophene, and another electrically conductive polymer. Furthermore, the hole injection/transport material is also suitable for use in an electron-blocking layer.

Specific examples of a compound that can be used as the hole injection/transport material include, but are not limited to, the following.

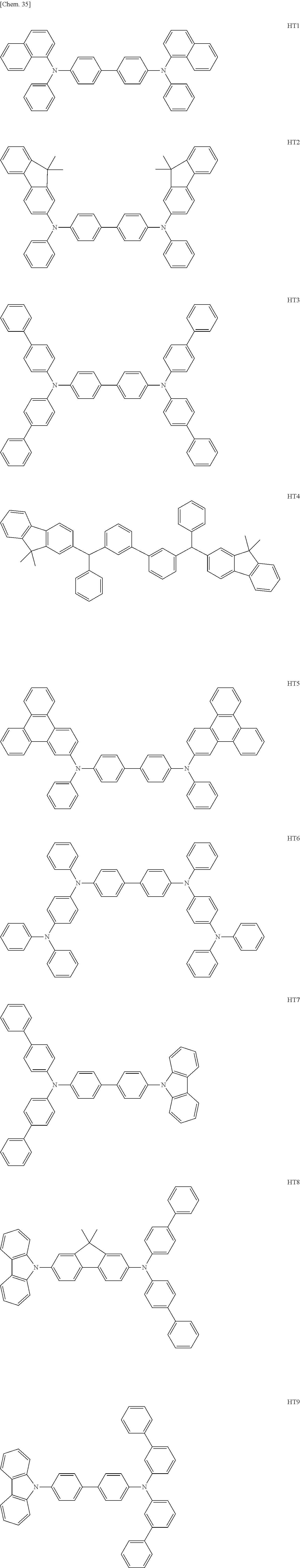

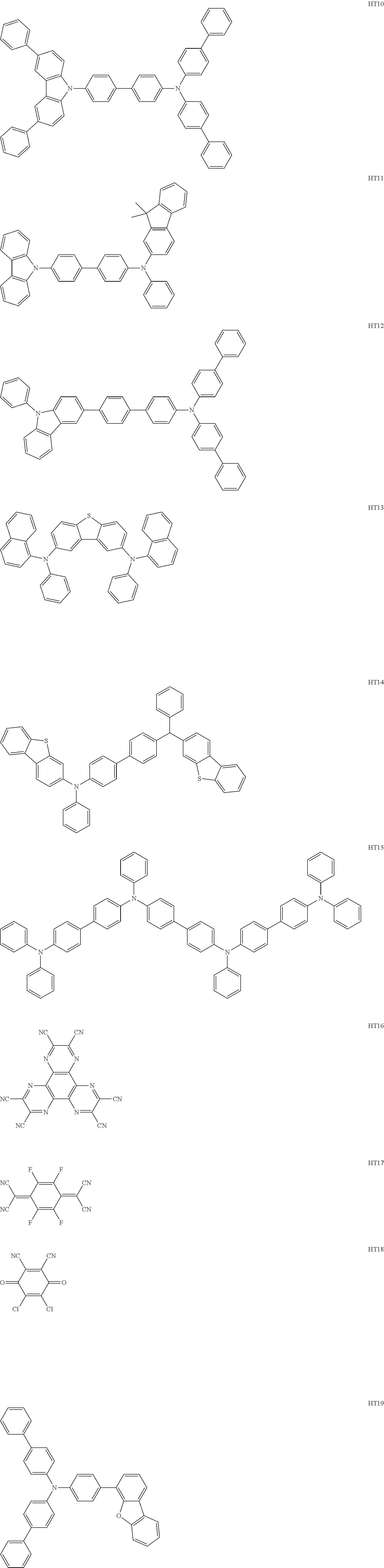

A light-emitting material mainly related to the light-emitting function may be, in addition to an organometallic complex represented by the general formula [2], a fused-ring compound (for example, a fluorene derivative, a naphthalene derivative, a pyrene derivative, a perylene derivative, a tetracene derivative, an anthracene derivative, rubrene, or the like), a quinacridone derivative, a coumarin derivative, a stilbene derivative, an organoaluminum complex, such as tris(8-quinolinolato)aluminum, an iridium complex, a platinum complex, a rhenium complex, a copper complex, an europium complex, a ruthenium complex, or a polymer derivative, such as a poly(phenylene vinylene) derivative, a polyfluorene derivative, or a polyphenylene derivative.

Specific examples of a compound that can be used as a light-emitting material include, but are not limited to, the following.

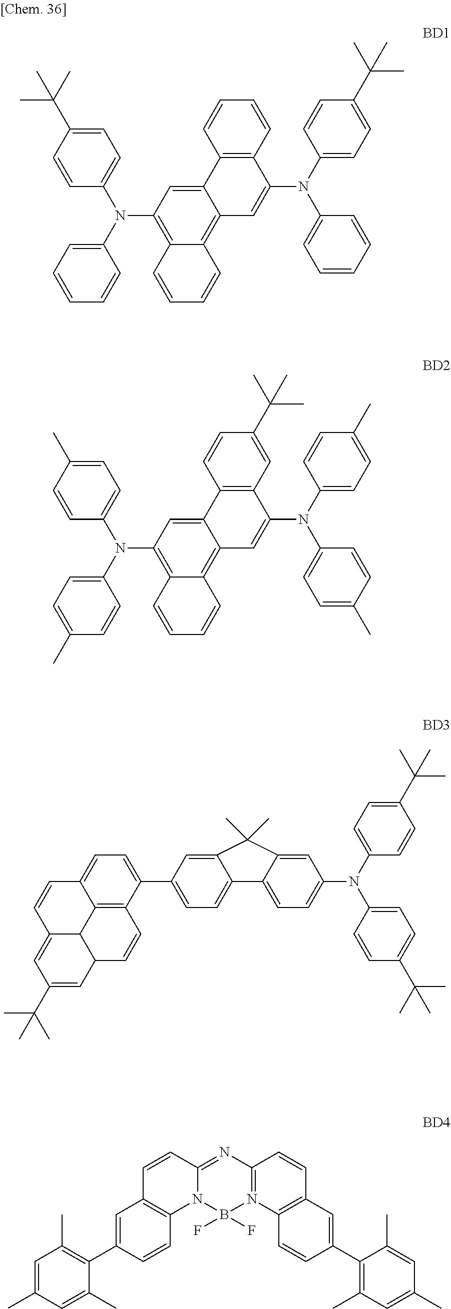

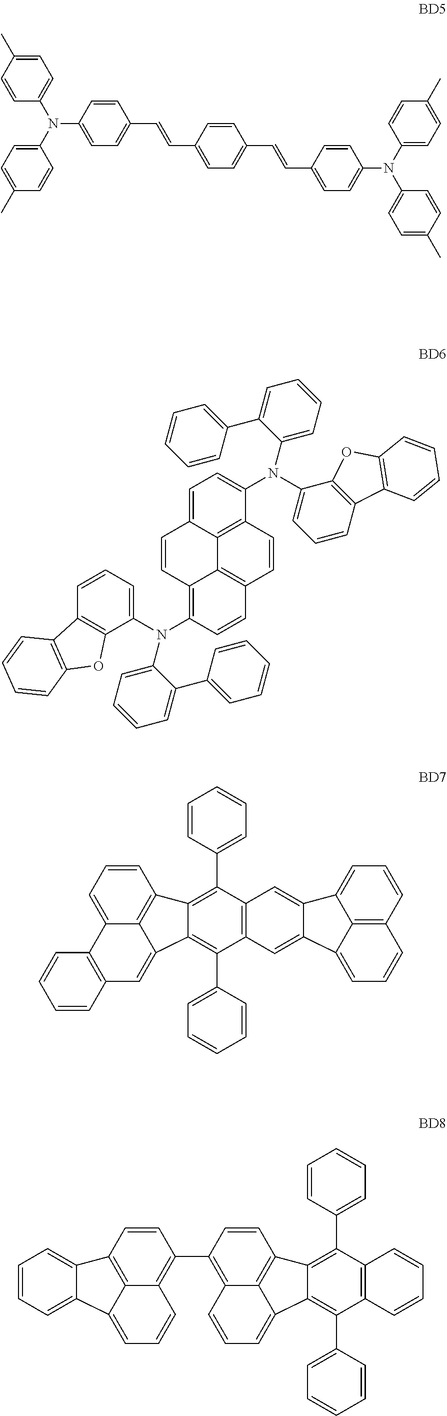

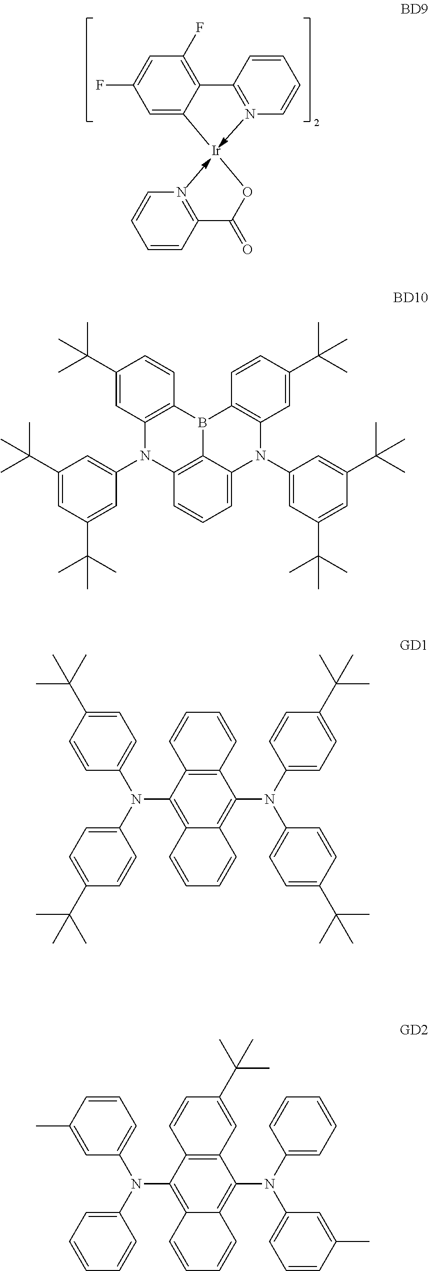

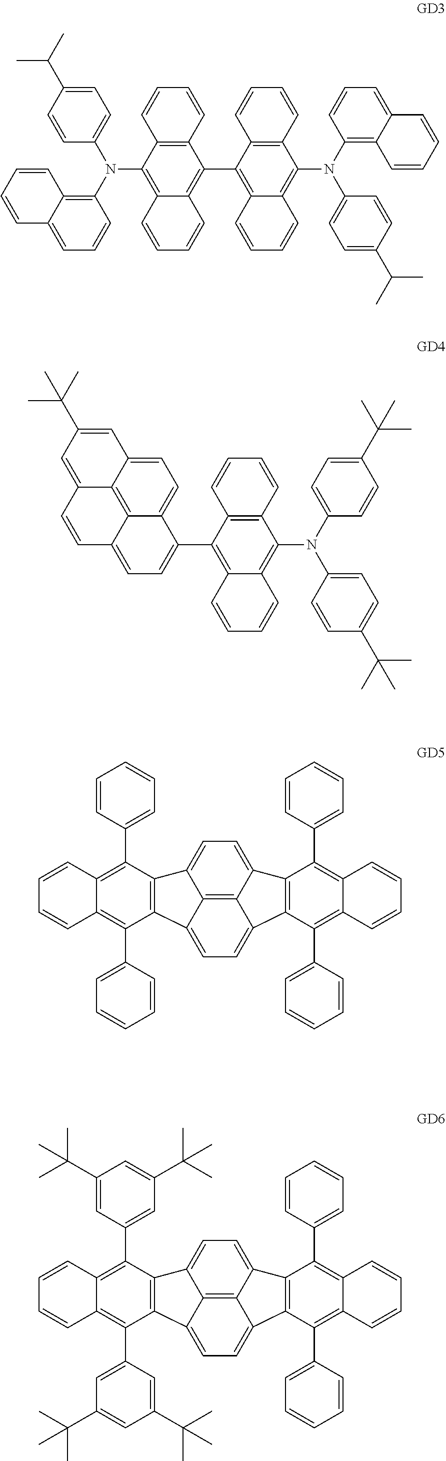

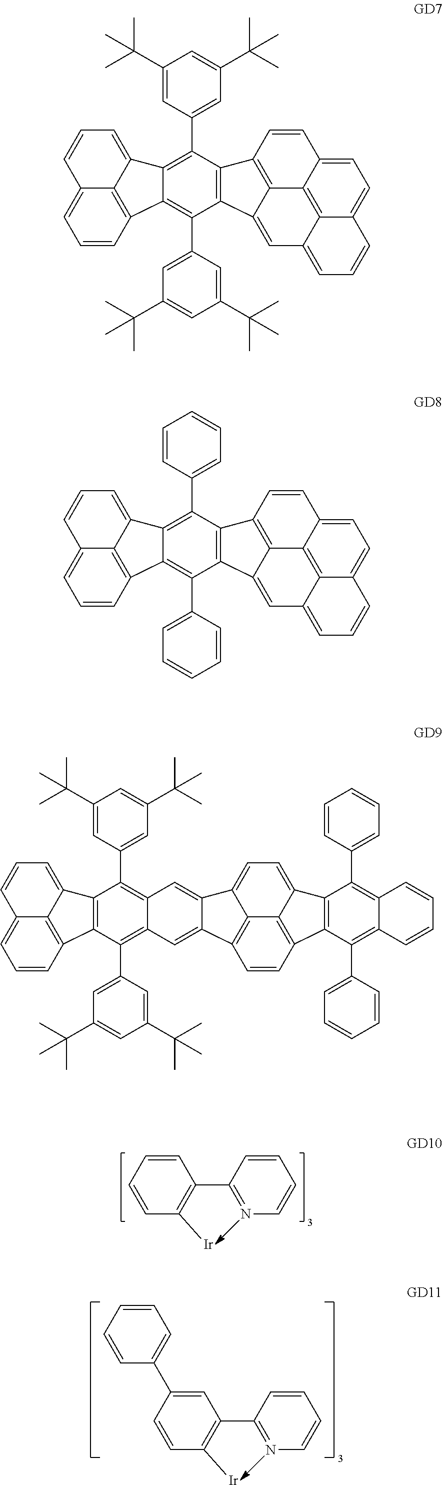

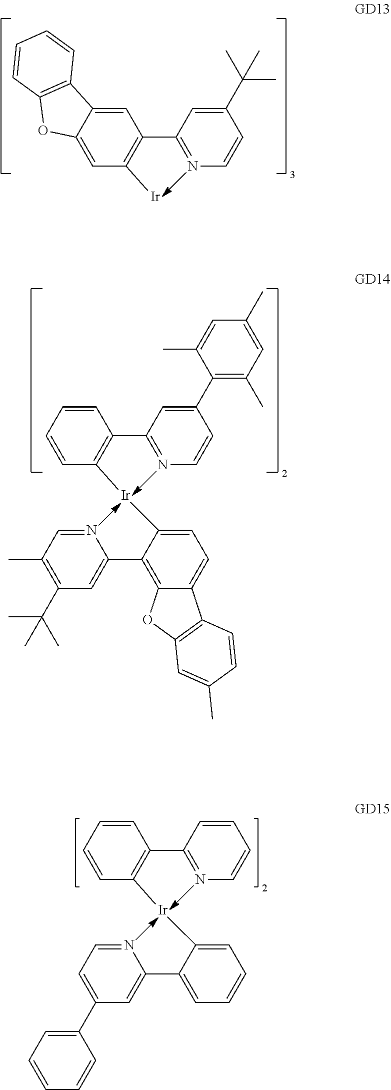

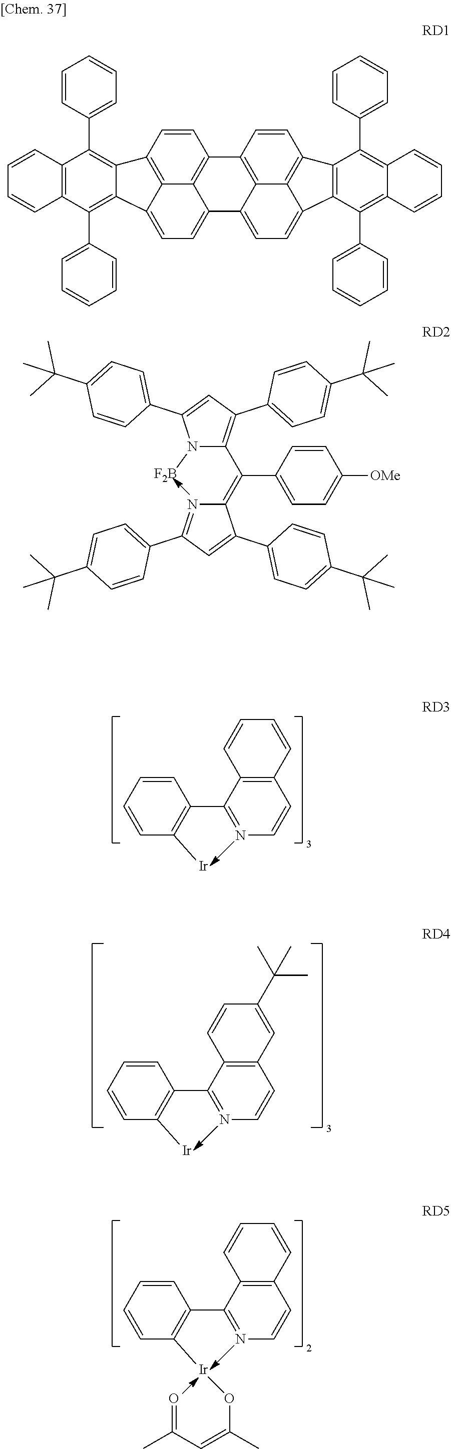

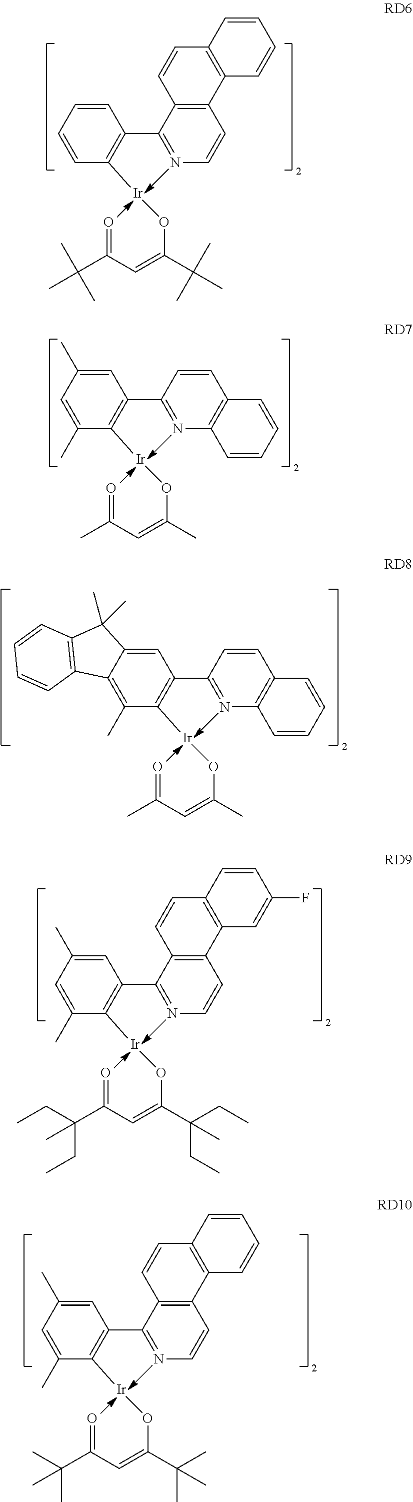

A host material or an assist material in a light-emitting layer may be, in addition to the materials of the exemplary compound A to D groups, an aromatic hydrocarbon compound or a derivative thereof, a carbazole derivative, a dibenzofuran derivative, a dibenzothiophene derivative, an organoaluminum complex, such as tris(8-quinolinolato)aluminum, an organoberyllium complex, or the like.

In particular, the assist material is preferably a material with a carbazole skeleton, a material with an azine ring, or a material with a xanthone skeleton. This is because these materials have high electron-donating ability and electron-withdrawing ability, and HOMO and LUMO can be easily adjusted. Such an assist material in combination with an organic compound according to the present invention can achieve a good carrier balance.

Specific examples of a compound that can be used as a light-emitting layer host or a light-emitting assist material in a light-emitting layer include, but are not limited to, the following.

In the following specific examples, the materials with the carbazole skeleton are EM32 to EM38. The materials with the azine ring are EM35 to EM40. The materials with the xanthone skeleton are EM28 and EM30.

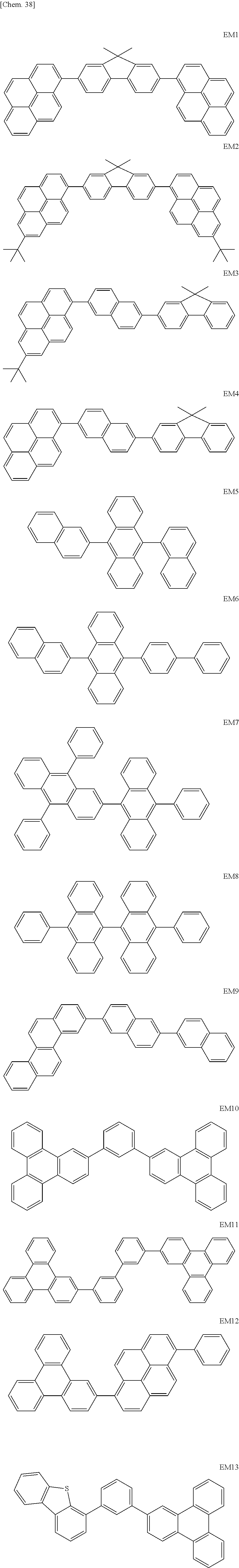

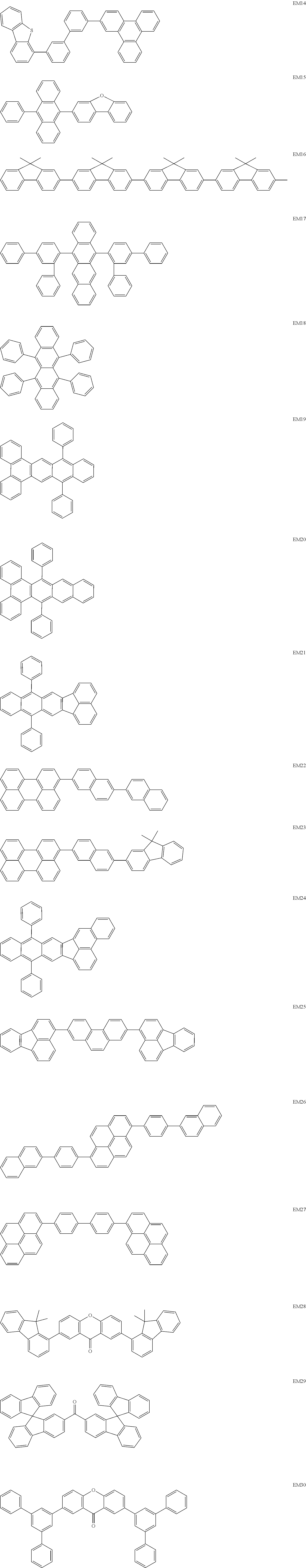

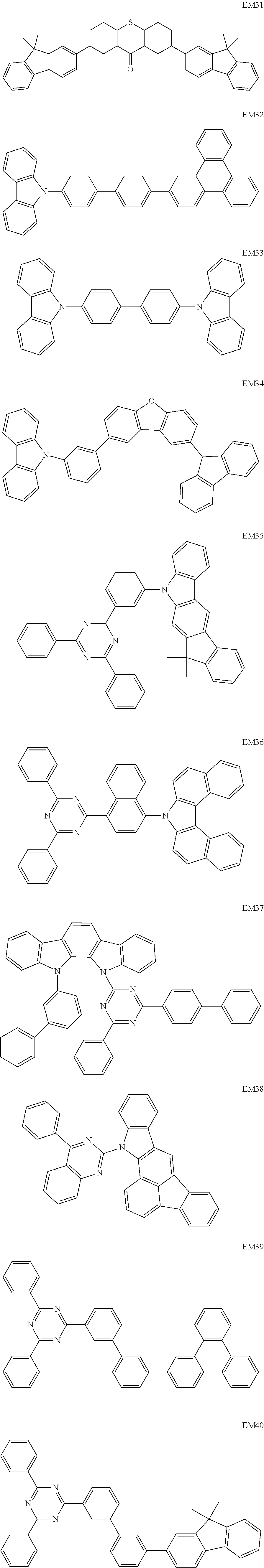

An electron transport material can be selected from materials that can transport an electron injected from the negative electrode to the light-emitting layer and is selected in consideration of the balance with the hole mobility of a hole transport material and the like. A material with electron transport ability may be an oxadiazole derivative, an oxazole derivative, a pyrazine derivative, a triazole derivative, a triazine derivative, a quinoline derivative, a quinoxaline derivative, a phenanthroline derivative, an organoaluminum complex, and a fused-ring compound (for example, a fluorene derivative, a naphthalene derivative, a chrysene derivative, an anthracene derivative, or the like). Furthermore, the electron transport material is also suitable for use in a hole-blocking layer.

Specific examples of a compound that can be used as the electron transport material include, but are not limited to, the following.

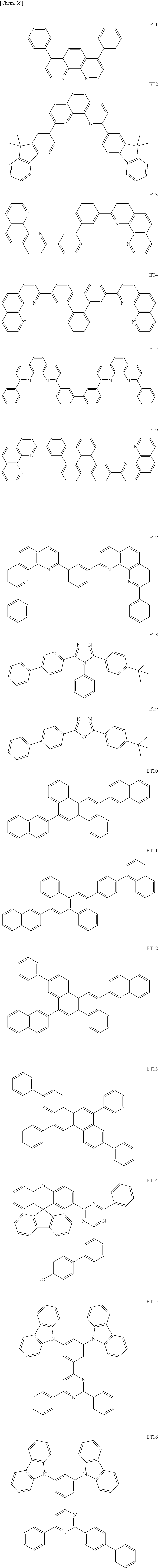

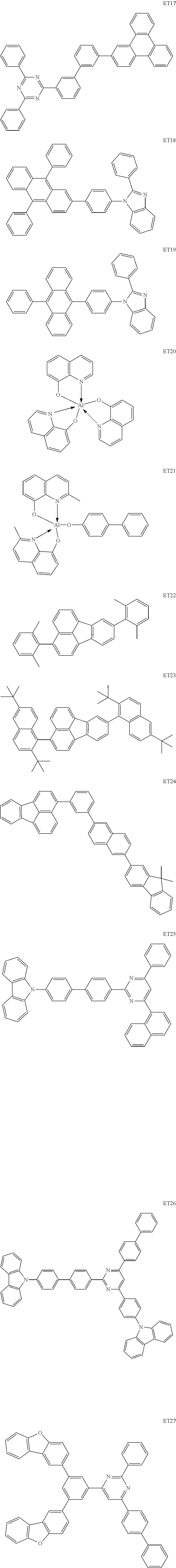

### (4) Structure of Organic Light-Emitting Device

Constituents other than the organic compound layer constituting the organic light-emitting device according to the present embodiment are described below.

An organic light-emitting device includes an insulating layer, a first electrode, an organic compound layer, and a second electrode on a substrate. A protective layer, a color filter, a microlens, or the like may be provided on the negative electrode. When a color filter is provided, a planarization layer may be provided between the color filter and the protective layer. The planarization layer may be composed of an acrylic resin or the like. The same applies to the planarization layer provided between the color filter and the microlens.

### [Substrate]

The substrate may be formed of quartz, glass, a silicon wafer, resin, metal, or the like. The substrate may have a switching element, such as a transistor, and wiring, on which an insulating layer may be provided. The insulating layer may be composed of any material, provided that the insulating layer can have a contact hole for wiring between the insulating layer and the first electrode and is insulated from unconnected wires. For example, the insulating layer may be formed of a resin, such as polyimide, silicon oxide, or silicon nitride.

### [Electrode]

A pair of electrodes can be used as electrodes. The pair of electrodes may be a positive electrode and a negative electrode. When an electric field is applied in a direction in which the organic light-emitting device emits light, an electrode with a high electric potential is a positive electrode, and the other electrode is a negative electrode. In other words, the electrode that supplies a hole to the light-emitting layer is a positive electrode, and the electrode that supplies an electron to the light-emitting layer is a negative electrode.

A constituent material of the positive electrode can have as large a work function as possible. Examples thereof include a metal element, such as gold, platinum, silver, copper, nickel, palladium, cobalt, selenium, vanadium, or tungsten, a mixture thereof, an alloy thereof, and a metal oxide, such as tin oxide, zinc oxide, indium oxide, indium tin oxide (ITO), or indium zinc oxide. An electrically conductive polymer, such as polyaniline, polypyrrole, or polythiophene, may also be used.

These electrode materials may be used alone or in combination. The positive electrode may be composed of a single layer or a plurality of layers.

When used as a reflective electrode, for example, chromium, aluminum, silver, titanium, tungsten, molybdenum, an alloy thereof, or a laminate thereof can be used. These materials can also function as a reflective film that does not have a role as an electrode. When used as a transparent electrode, an oxide transparent electroconductive layer, such as indium tin oxide (ITO) or indium zinc oxide, can be used. However, the present invention is not limited thereto. The electrodes may be formed by photolithography.

On the other hand, a constituent material of the negative electrode can be a material with a small work function. For example, an alkali metal, such as lithium, an alkaline-earth metal, such as calcium, a metal element, such as aluminum, titanium, manganese, silver, lead, or chromium, or a mixture thereof may be used. An alloy of these metal elements may also be used. For example, magnesium-silver, aluminum-lithium, aluminum-magnesium, silver-copper, or zinc-silver may be used. A metal oxide, such as indium tin oxide (ITO), may also be used. These electrode materials may be used alone or in combination. The negative electrode may be composed of a single layer or a plurality of layers. In particular, silver is preferably used, and a silver alloy is more preferably used to reduce the aggregation of silver. As long as the aggregation of silver can be reduced, the alloy may have any ratio. For example, the ratio of silver to another metal may be 1:1, 3:1, or the like.

The negative electrode may be, but is not limited to, an oxide electroconductive layer, such as ITO, for a top emission device or a reflective electrode, such as aluminum (Al), for a bottom emission device. The negative electrode may be formed by any method. A direct-current or alternating-current sputtering method can achieve good film coverage and easily decrease resistance.

### [Organic Compound Layer]

The organic compound layer may be formed of a single layer or a plurality of layers. Depending on their functions, the plurality of layers may be referred to as a hole injection layer, a hole transport layer, an electron-blocking layer, a light-emitting layer, a hole-blocking layer, an electron transport layer, or an electron injection layer. The organic compound layer is composed mainly of an organic compound and may contain an inorganic atom or an inorganic compound. For example, copper, lithium, magnesium, aluminum, iridium, platinum, molybdenum, zinc, or the like may be contained. The organic compound layer may be located between the first electrode and the second electrode and may be in contact with the first electrode and the second electrode.

### [Protective Layer]

A protective layer may be provided on the negative electrode. For example, a glass sheet with a moisture absorbent may be attached to the negative electrode to decrease the amount of water or the like entering the organic compound layer and to reduce the occurrence of display defects. In another embodiment, a passivation film of silicon nitride or the like may be provided on the negative electrode to decrease the amount of water or the like entering the organic compound layer. For example, the negative electrode may be formed and then transferred to another chamber without breaking the vacuum, and a silicon nitride film with a thickness of 2 μm may be formed as a protective layer by a chemical vapor deposition (CVD) method. The film formation by the CVD method may be followed by the formation of a protective layer by an atomic layer deposition (ALD) method. A film formed by the ALD method may be formed of any material, such as silicon nitride, silicon oxide, or aluminum oxide. Silicon nitride may be further formed by the CVD method on the film formed by the ALD method. The film formed by the ALD method may have a smaller thickness than the film formed by the CVD method. More specifically, the thickness may be 50% or less or even 10% or less.

### [Color Filter]

A color filter may be provided on the protective layer. For example, a color filter that matches the size of the organic light-emitting device may be provided on another substrate and may be bonded to the substrate on which the organic light-emitting device is provided, or a color filter may be patterned on the protective layer by photolithography. The color filter may be composed of a polymer.

### [Planarization Layer]

A planarization layer may be provided between the color filter and the protective layer. The planarization layer is provided to reduce the roughness of the underlayer. The planarization layer is sometimes referred to as a material resin layer with any purpose. The planarization layer may be composed of an organic compound and is preferably composed of a high-molecular-weight compound, though it may be composed of a low-molecular-weight compound.

The planarization layer may be provided above and below the color filter, and the constituent materials thereof may be the same or different. Specific examples include a polyvinylcarbazole resin, a polycarbonate resin, a polyester resin, an ABS resin, an acrylic resin, a polyimide resin, a phenolic resin, an epoxy resin, a silicone resin, and a urea resin.

### [Microlens]

An organic light-emitting apparatus may include an optical member, such as a microlens, on the light output side. The microlens may be composed of an acrylic resin, an epoxy resin, or the like. The microlens may be used to increase the amount of light extracted from the organic light-emitting apparatus and to control the direction of the extracted light. The microlens may have a hemispherical shape. For a hemispherical microlens, the vertex of the microlens is a contact point between the hemisphere and a tangent line parallel to the insulating layer among the tangent lines in contact with the hemisphere. The vertex of the microlens in any cross-sectional view can be determined in the same manner. More specifically, the vertex of the microlens in a cross-sectional view is a contact point between the semicircle of the microlens and a tangent line parallel to the insulating layer among the tangent lines in contact with the semicircle.

The midpoint of the microlens can also be defined. In a cross section of the microlens, a midpoint of a line segment from one end point to the other end point of the arc can be referred to as a midpoint of the microlens. A cross section in which the vertex and the midpoint are determined may be perpendicular to the insulating layer.

### [Opposite Substrate]

An opposite substrate may be provided on the planarization layer. The opposite substrate is so called because it faces the substrate. The opposite substrate may be composed of the same material as the substrate. When the substrate is a first substrate, the opposite substrate may be a second substrate.

### [Organic Layer]

An organic compound layer (a hole injection layer, a hole transport layer, an electron-blocking layer, a light-emitting layer, a hole-blocking layer, an electron transport layer, an electron injection layer, or the like) constituting an organic light-emitting device according to an embodiment of the present invention is formed by the following methods.

An organic compound layer constituting an organic light-emitting device according to an embodiment of the present invention can be formed by a dry process, such as a vacuum deposition method, an ionized deposition method, sputtering, or plasma. Instead of the dry process, a wet process may also be employed in which a layer is formed by a known coating method (for example, spin coating, dipping, a casting method, an LB method, an ink jet method, or the like) using an appropriate solvent.

A layer formed by a vacuum deposition method, a solution coating method, or the like undergoes little crystallization or the like and has high temporal stability. When a film is formed by a coating method, the film may also be formed in combination with an appropriate binder resin.

The binder resin may be, but is not limited to, a polyvinylcarbazole resin, a polycarbonate resin, a polyester resin, an ABS resin, an acrylic resin, a polyimide resin, a phenolic resin, an epoxy resin, a silicone resin, or a urea resin.

These binder resins may be used alone or in combination as a homopolymer or a copolymer. If necessary, an additive agent, such as a known plasticizer, oxidation inhibitor, and/or ultraviolet absorbent, may also be used.

### [Pixel Circuit]

A light-emitting apparatus may include a pixel circuit coupled to the light-emitting device. The pixel circuit may be of an active-matrix type, which independently controls the light emission of a first light-emitting device and a second light-emitting device. The active-matrix circuit may be voltage programmed or current programmed. The drive circuit has a pixel circuit for each pixel. The pixel circuit may include a light-emitting device, a transistor for controlling the luminous brightness of the light-emitting device, a transistor for controlling light emission timing, a capacitor for holding the gate voltage of the transistor for controlling the luminous brightness, and a transistor for GND connection without through the light-emitting device.

A light-emitting apparatus includes a display region and a peripheral region around the display region. The display region includes the pixel circuit, and the peripheral region includes a display control circuit. The mobility of a transistor constituting the pixel circuit may be smaller than the mobility of a transistor constituting the display control circuit.

The gradient of the current-voltage characteristics of a transistor constituting the pixel circuit may be smaller than the gradient of the current-voltage characteristics of a transistor constituting the display control circuit. The gradient of the current-voltage characteristics can be determined by so-called Vg-Ig characteristics.

A transistor constituting the pixel circuit is a transistor coupled to a light-emitting device, such as a first light-emitting device.

### [Pixel]

An organic light emitting apparatus has a plurality of pixels. Each pixel has subpixels that emit light of different colors. For example, the subpixels may have RGB emission colors.

In each pixel, a region also referred to as a pixel aperture emits light. This region is the same as the first region. The pixel aperture may be 15 μm or less or 5 μm or more. More specifically, the pixel aperture may be 11 μm, 9.5 μm, 7.4 μm, or 6.4 μm.

The distance between the subpixels may be 10 μm or less, more specifically, 8 μm, 7.4 μm, or 6.4 μm.

The pixels may be arranged in a known form in a plan view. Examples include a stripe arrangement, a delta arrangement, a PenTile arrangement, and a Bayer arrangement. Each subpixel may have any known shape in a plan view. Examples include quadrangles, such as a rectangle and a rhombus, and a hexagon. As a matter of course, the rectangle also includes a figure that is not strictly rectangular but is close to rectangular. The shape of each subpixel and the pixel array can be used in combination.

### (5) Applications of Organic Light-Emitting Device According to Present Embodiment

An organic light-emitting device according to an embodiment of the present invention can be used as a constituent of a display apparatus or a lighting apparatus. Other applications include an exposure light source for an electrophotographic image-forming apparatus, a backlight for a liquid crystal display, and a light-emitting apparatus with a color filter in a white light source.

The display apparatus may be an image-information-processing apparatus that includes an image input unit for inputting image information from an area CCD, a linear CCD, a memory card, or the like, includes an information processing unit for processing the input information, and displays an input image on a display unit.

A display unit of an imaging apparatus or an ink jet printer may have a touch panel function. A driving system of the touch panel function may be, but is not limited to, an infrared radiation system, an electrostatic capacitance system, a resistive film system, or an electromagnetic induction system. The display apparatus may be used for a display unit of a multifunction printer.

Next, the display apparatus according to the present embodiment is described below with reference to the accompanying drawings.

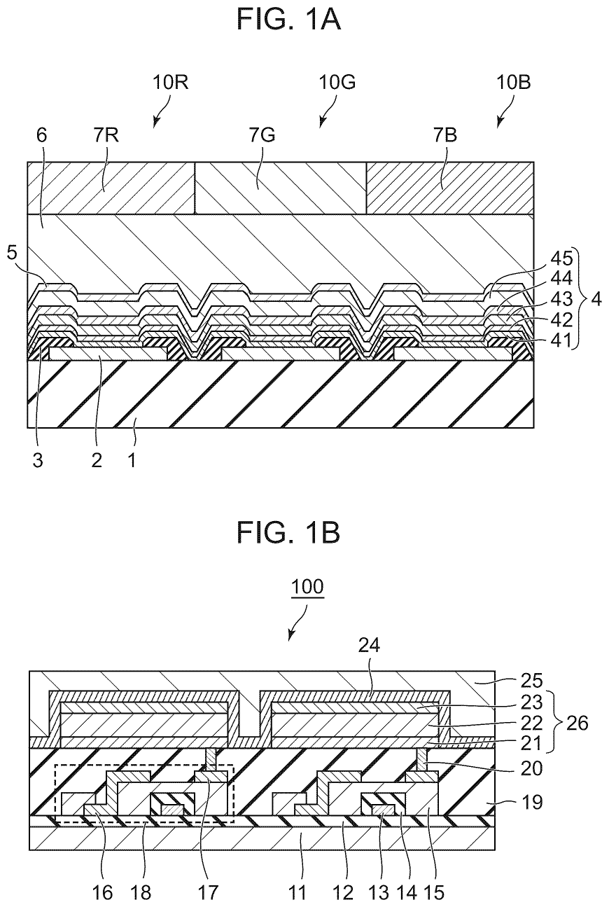

FIGS. 1A and 1B are schematic cross-sectional views of an example of a display apparatus that includes an organic light-emitting device and a transistor coupled to the organic light-emitting device. The transistor is an example of an active device. The transistor may be a thin-film transistor (TFT).

FIG. 1A illustrates an example of a pixel serving as a constructional device of the display apparatus according to the present embodiment. The pixel has subpixels 10. The subpixels are 10R, 10G, and 10B with different emission colors. The emission colors may be distinguished by the wavelength of light emitted from the light-emitting layer, or light emitted from each subpixel may be selectively transmitted or color-converted with a color filter or the like. Each subpixel has, on an interlayer insulating layer 1, a reflective electrode 2 as a first electrode, an insulating layer 3 covering the ends of the reflective electrode 2, organic compound layers 4 covering the first electrode and the insulating layer, a transparent electrode 5, a protective layer 6, and a color filter 7.

A transistor and/or a capacitor element may be provided under or inside the interlayer insulating layer 1. The transistor may be electrically connected to the first electrode via a contact hole (not shown) or the like.

The insulating layer 3 is also referred to as a bank or a pixel separation film. The insulating layer 3 covers the ends of the first electrode and surrounds the first electrode. A portion of the first electrode not covered with the insulating layer is in contact with the organic compound layers 4 and serves as a light-emitting region.

The organic compound layers 4 include a hole injection layer 41, a hole transport layer 42, a first light-emitting layer 43, a second light-emitting layer 44, and an electron transport layer 45.

The second electrode 5 may be a transparent electrode, a reflective electrode, or a semitransparent electrode.

The protective layer 6 reduces the penetration of moisture into the organic compound layers. The protective layer is illustrated as a single layer but may be a plurality of layers. The protective layer 6 may include an inorganic compound layer and an organic compound layer.

The color filter 7 is divided into 7R, 7G, and 7B according to the color. The color filter may be formed on a planarizing film (not shown). Furthermore, a resin protective layer (not shown) may be provided on the color filter. The color filter may be formed on the protective layer 6. Alternatively, the color filter may be bonded after being provided on an opposite substrate, such as a glass substrate.

A display apparatus 100 in FIG. 1B includes an organic light-emitting device 26 and a TFT 18 as an example of a transistor. The display apparatus includes a substrate 11 made of glass, silicon, or the like and an insulating layer 12 on the substrate 11. The display apparatus 100 includes, on the insulating layer, an active element 18, such as a TFT, and a gate electrode 13, a gate-insulating film 14, and a semiconductor layer 15 of the active element. The TFT 18 is also composed of the semiconductor layer 15, a drain electrode 16, and a source electrode 17. The TFT 18 is covered with an insulating film 19. A positive electrode 21 of the organic light-emitting device 26 is coupled to the source electrode 17 through a contact hole 20 formed in the insulating film.

The method for electrically connecting the electrodes (the positive electrode and a negative electrode) of the organic light-emitting device 26 to the electrodes (the source electrode and the drain electrode) of the TFT is not limited to the embodiment illustrated in FIG. 1B. More specifically, it is only necessary to electrically connect one of the positive electrode and the negative electrode to one of the source electrode and the drain electrode of the TFT. TFT refers to a thin-film transistor.

The organic compound layer 22 in the display apparatus 100 in FIG. 1B is a single layer but may be composed of a plurality of layers. The negative electrode 23 is covered with a first protective layer 24 and a second protective layer 25 for reducing degradation of the organic light-emitting device.

The display apparatus 100 in FIG. 1B includes a transistor as a switching element but may include another switching element instead thereof.

The transistor used in the display apparatus 100 in FIG. 1B is not limited to a transistor including a single crystal silicon wafer and may also be a thin-film transistor including an active layer on an insulating surface of a substrate. The active layer may be single-crystal silicon, non-single-crystal silicon, such as amorphous silicon or microcrystalline silicon, or a non-single-crystal oxide semiconductor, such as indium zinc oxide or indium gallium zinc oxide. The thin-film transistor is also referred to as a TFT element.

The transistor in the display apparatus 100 in FIG. 1B may be formed within a substrate, such as a Si substrate. The phrase “formed within a substrate” means that the substrate, such as a Si substrate, itself is processed to form the transistor. Thus, the transistor within the substrate can be considered that the substrate and the transistor are integrally formed.

In the organic light-emitting device according to the present embodiment, the luminous brightness is controlled with the TFT, which is an example of a switching element. The organic light-emitting device can be provided in a plurality of planes to display an image at each luminous brightness. The switching element according to the present embodiment is not limited to the TFT and may be a transistor formed of low-temperature polysilicon or an active-matrix driver formed on a substrate, such as a Si substrate. The phrase “on a substrate” may also be referred to as “within a substrate”. Whether a transistor is provided within a substrate or a TFT is used depends on the size of a display unit. For example, for an approximately 0.5-inch display unit, an organic light-emitting device is preferably provided on a Si substrate.

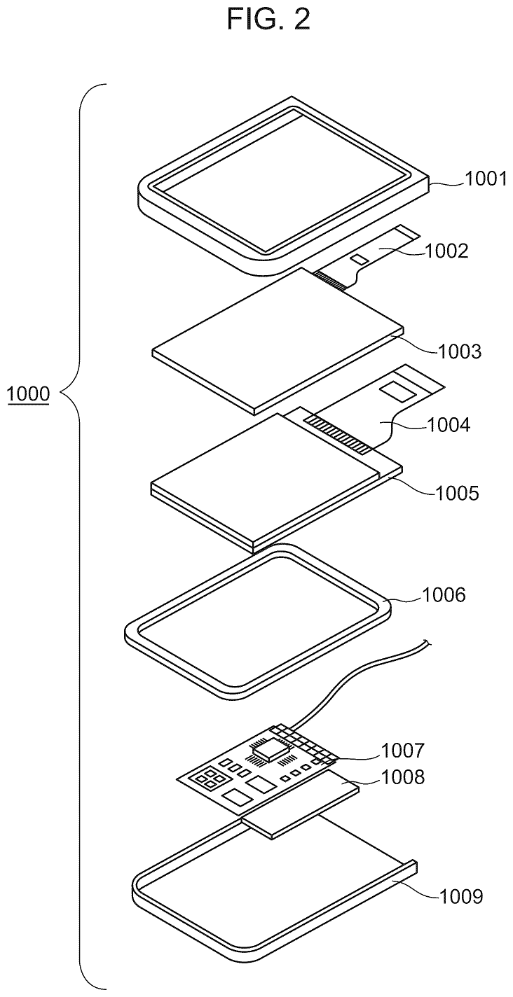

FIG. 2 is a schematic view of an example of the display apparatus according to the present embodiment. A display apparatus 1000 may include a touch panel 1003, a display panel 1005, a frame 1006, a circuit substrate 1007, and a battery 1008 between an upper cover 1001 and a lower cover 1009. The touch panel 1003 and the display panel 1005 are coupled to flexible print circuits FPC 1002 and 1004, respectively. Transistors are printed on the circuit substrate 1007. The battery 1008 is not necessarily provided when the display apparatus is not a mobile device, or may be provided at another position even when the display apparatus is a mobile device.

The display apparatus according to the present embodiment may include color filters of red, green, and blue colors. In the color filters, the red, green, and blue colors may be arranged in a delta arrangement.

The display apparatus according to the present embodiment may be used for a display unit of a mobile terminal. Such a display apparatus may have both a display function and an operation function. The mobile terminal may be a mobile phone, such as a smartphone, a tablet, a head-mounted display, or the like.

The display apparatus according to the present embodiment may be used for a display unit of an imaging apparatus that includes an optical unit with a plurality of lenses and an imaging device for receiving light passing through the optical unit. The imaging apparatus may include a display unit for displaying information acquired by the imaging device. The display unit may be a display unit exposed outside from the imaging apparatus or a display unit located in a finder. The imaging apparatus may be a digital camera or a digital camcorder.

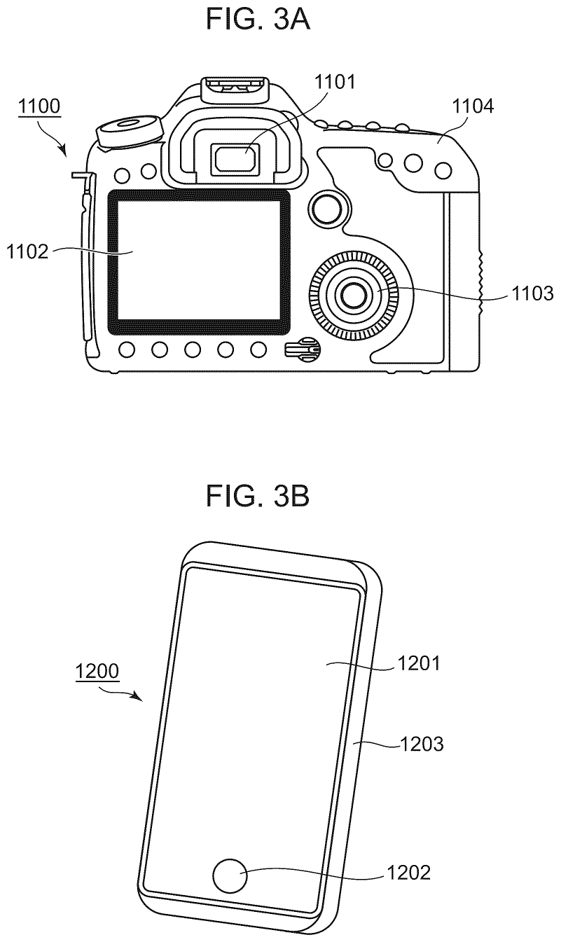

FIG. 3A is a schematic view of an example of an imaging apparatus according to the present embodiment. An imaging apparatus 1100 may include a viewfinder 1101, a rear display 1102, an operating unit 1103, and a housing 1104. The viewfinder 1101 may include the display apparatus according to the present embodiment. In such a case, the display apparatus may display environmental information, imaging instructions, and the like as well as an image to be captured. The environmental information may include the intensity of external light, the direction of external light, the travel speed of the photographic subject, the possibility that the photographic subject is shielded by a shielding material, and the like.

Because the appropriate timing for imaging is a short time, it is better to display information as early as possible. Thus, a display apparatus including an organic light-emitting device according to the present invention is preferably used. This is because the organic light-emitting device has a high response speed. A display apparatus including the organic light-emitting device can be more suitably used than these apparatuses and liquid crystal displays that require a high display speed.

The imaging apparatus 1100 includes an optical unit (not shown). The optical unit has a plurality of lenses and focuses an image on an imaging device in the housing 1104. The focus of the lenses can be adjusted by adjusting their relative positions. This operation can also be automatically performed. The imaging apparatus may also be referred to as a photoelectric conversion apparatus. The photoelectric conversion apparatus can have, as an imaging method, a method of detecting a difference from a previous image, a method of cutting out a permanently recorded image, or the like, instead of taking an image one after another.

FIG. 3B is a schematic view of an example of electronic equipment according to the present embodiment. Electronic equipment 1200 includes a display unit 1201, an operating unit 1202, and a housing 1203. The housing 1203 may include a circuit, a printed circuit board including the circuit, a battery, and a communication unit. The operating unit 1202 may be a button or a touch panel response unit. The operating unit may be a biometric recognition unit that recognizes a fingerprint and releases the lock. Electronic equipment with a communication unit may also be referred to as communication equipment. The electronic equipment may have a lens and an imaging device and thereby further have a camera function. An image captured by the camera function is displayed on the display unit. The electronic equipment may be a smartphone, a notebook computer, or the like.

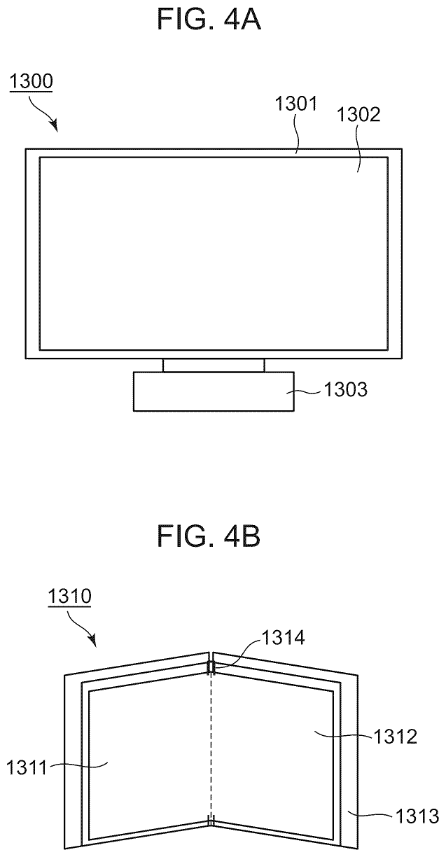

FIGS. 4A and 4B are schematic views of an example of the display apparatus according to the present embodiment. FIG. 4A illustrates a display apparatus, such as a television monitor or a PC monitor. A display apparatus 1300 includes a frame 1301 and a display unit 1302. A light-emitting apparatus according to the present embodiment may be used for the display unit 1302.

The frame 1301 and the display unit 1302 are supported by a base 1303. The base 1303 is not limited to the structure illustrated in FIG. 4A. The lower side of the frame 1301 may also serve as the base.

The frame 1301 and the display unit 1302 may be bent. The radius of curvature thereof may be 5000 mm or more and 6000 mm or less.

FIG. 4B is a schematic view of another example of the display apparatus according to the present embodiment. A display apparatus 1310 in FIG. 4B is configured to be foldable and is a so-called foldable display apparatus. The display apparatus 1310 includes a first display unit 1311, a second display unit 1312, a housing 1313, and a folding point 1314. The first display unit 1311 and the second display unit 1312 may include the light-emitting apparatus according to the present embodiment. The first display unit 1311 and the second display unit 1312 may be a single display apparatus without a joint. The first display unit 1311 and the second display unit 1312 can be divided by the folding point. The first display unit 1311 and the second display unit 1312 may display different images or one image.

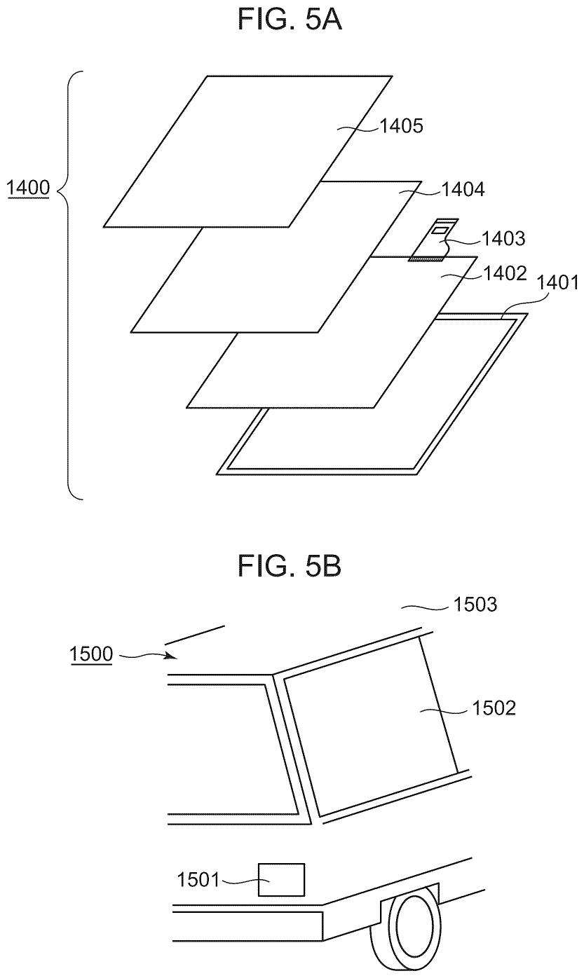

FIG. 5A is a schematic view of an example of a lighting apparatus according to the present embodiment. A lighting apparatus 1400 may include a housing 1401, a light source 1402, a circuit substrate 1403, an optical film 1404, and a light-diffusing unit 1405. The light source may include the organic light-emitting device according to the present embodiment. The optical filter may be a filter for improving the color rendering properties of the light source. The light-diffusing unit can effectively diffuse light from the light source and widely spread light as in illumination. The optical filter and the light-diffusing unit may be provided on the light output side of the lighting apparatus. If necessary, a cover may be provided on the outermost side.

For example, the lighting apparatus is an interior lighting apparatus. The lighting apparatus may emit white light, neutral white light, or light of any color from blue to red. The lighting apparatus may have a light control circuit for controlling such light. The lighting apparatus may include an organic light-emitting device according to the present invention and a power supply circuit coupled thereto. The power supply circuit is a circuit that converts an AC voltage to a DC voltage. White has a color temperature of 4200 K, and neutral white has a color temperature of 5000 K. The lighting apparatus may have a color filter.

The lighting apparatus according to the present embodiment may include a heat dissipation unit. The heat dissipation unit releases heat from the apparatus to the outside and may be a metal or liquid silicon with a high specific heat.

FIG. 5B is a schematic view of an automobile as an example of a moving body according to the present embodiment. The automobile has a taillight as an example of a lamp. An automobile 1500 may have a taillight 1501, which comes on when a brake operation or the like is performed.

The taillight 1501 may include the organic light-emitting device according to the present embodiment. The taillight may have a protective member for protecting an organic EL device. The protective member may be formed of any transparent material with moderately high strength and is preferably formed of polycarbonate or the like. The polycarbonate may be mixed with a furan dicarboxylic acid derivative, an acrylonitrile derivative, or the like.

The automobile 1500 may have a body 1503 and a window 1502 on the body 1503. The window may be a transparent display as long as it is not a window for checking the front and rear of the automobile. The transparent display may include the organic light-emitting device according to the present embodiment. In such a case, constituent materials, such as electrodes, of the organic light-emitting device are transparent materials.

The moving body according to the present embodiment may be a ship, an aircraft, a drone, or the like. The moving body may include a body and a lamp provided on the body. The lamp may emit light to indicate the position of the body. The lamp includes the organic light-emitting device according to the present embodiment.

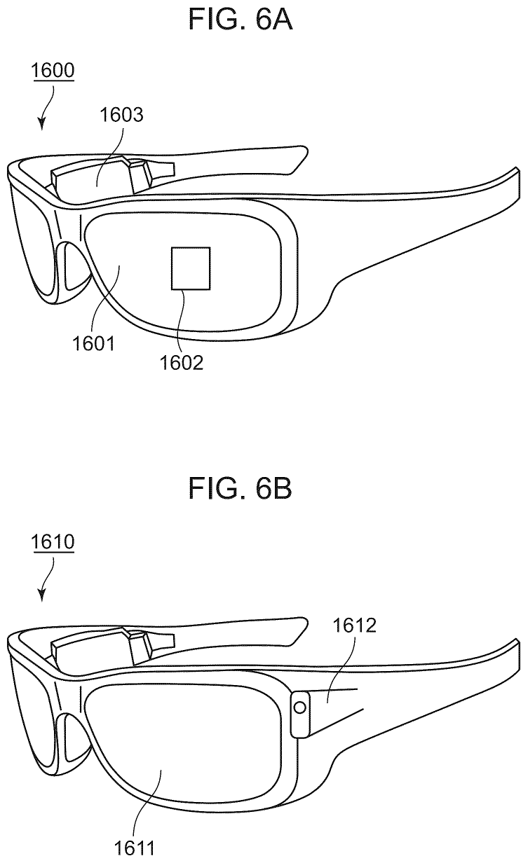

Application examples of the display apparatus according to one of the embodiments are described below with reference to FIGS. 6A and 6B. The display apparatus can be applied to a system that can be worn as a wearable device, such as smart glasses, a head-mounted display (HMD), or smart contact lenses. An imaging and displaying apparatus used in such an application example includes an imaging apparatus that can photoelectrically convert visible light and a display apparatus that can emit visible light.

FIG. 6A illustrates glasses 1600 (smart glasses) according to one application example. An imaging apparatus 1602, such as a complementary metal-oxide semiconductor (CMOS) sensor or a single-photon avalanche photodiode (SPAD), is provided on the front side of a lens 1601 of the glasses 1600. The display apparatus according to one of the embodiments is provided on the back side of the lens 1601.

The glasses 1600 further include a controller 1603. The controller 1603 functions as a power supply for supplying power to the imaging apparatus 1602 and the display apparatus according to one of the embodiments. The controller 1603 controls the operation of the imaging apparatus 1602 and the display apparatus. The lens 1601 has an optical system for focusing light on the imaging apparatus 1602.

FIG. 6B illustrates glasses 1610 (smart glasses) according to one application example. The glasses 1610 have a controller 1612, which includes an imaging apparatus corresponding to the imaging apparatus 1602 and a display apparatus. A lens 1611 includes an optical system for projecting light from the imaging apparatus and the display apparatus of the controller 1612, and an image is projected on the lens 1611. The controller 1612 functions as a power supply for supplying power to the imaging apparatus and the display apparatus and controls the operation of the imaging apparatus and the display apparatus. The controller may include a line-of-sight detection unit for detecting the line of sight of the wearer. Infrared radiation may be used to detect the line of sight. An infrared radiation unit emits infrared light to an eyeball of a user who is gazing at a display image. Reflected infrared light from the eyeball is detected by an imaging unit including a light-receiving device to capture an image of the eyeball. A reduction unit for reducing light from the infrared radiation unit to a display unit in a plan view is provided to reduce degradation in image quality.

The line of sight of the user for the display image is detected from the image of the eyeball captured by infrared imaging. Any known technique can be applied to line-of-sight detection using the captured image of the eyeball. For example, it is possible to use a line-of-sight detection method based on a Purkinje image obtained by the reflection of irradiation light by the cornea.

More specifically, a line-of-sight detection process based on a pupil-corneal reflection method is performed. The line of sight of the user is detected by calculating a line-of-sight vector representing the direction (rotation angle) of an eyeball on the basis of an image of a pupil and a Purkinje image included in a captured image of the eyeball using the pupil-corneal reflection method.

A display apparatus according to an embodiment of the present invention may include an imaging apparatus including a light-receiving device and may control a display image on the basis of line-of-sight information of a user from the imaging apparatus.

More specifically, on the basis of the line-of-sight information, the display apparatus determines a first visibility region at which the user gazes and a second visibility region other than the first visibility region. The first visibility region and the second visibility region may be determined by the controller of the display apparatus or may be received from an external controller. In the display region of the display apparatus, the first visibility region may be controlled to have higher display resolution than the second visibility region. In other words, the second visibility region may have lower resolution than the first visibility region.

The display region has a first display region and a second display region different from the first display region, and the priority of the first display region and the second display region depends on the line-of-sight information. The first visibility region and the second visibility region may be determined by the controller of the display apparatus or may be received from an external controller. A region with a higher priority may be controlled to have higher resolution than another region. In other words, a region with a lower priority may have lower resolution.

The first visibility region or a region with a higher priority may be determined by artificial intelligence (AI). The AI may be a model configured to estimate the angle of the line of sight and the distance to a target ahead of the line of sight from an image of an eyeball using the image of the eyeball and the direction in which the eyeball actually viewed in the image as teaching data. The AI program may be stored in the display apparatus, the imaging apparatus, or an external device. The AI program stored in an external device is transmitted to the display apparatus via communication.

For display control based on visual recognition detection, the present invention can be applied to smart glasses further having an imaging apparatus for imaging the outside. Smart glasses can display captured external information in real time.

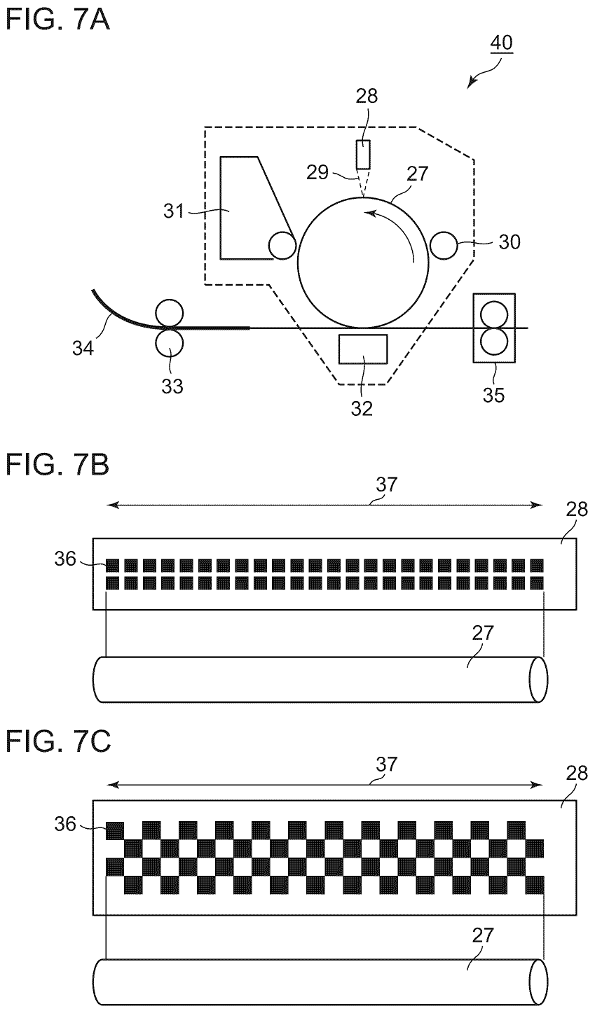

FIG. 7A is a schematic view of an example of an image-forming apparatus according to an embodiment of the present invention. An image-forming apparatus 40 is an electrophotographic image-forming apparatus and includes a photosensitive member 27, an exposure light source 28, a charging unit 30, a developing unit 31, a transfer unit 32, a conveying roller 33, and a fixing unit 35. The exposure light source 28 emits light 29, and an electrostatic latent image is formed on the surface of the photosensitive member 27. The exposure light source 28 includes the organic light-emitting device according to the present embodiment. The developing unit 31 contains toner and the like. The charging unit 30 electrifies the photosensitive member 27. The transfer unit 32 transfers a developed image onto a recording medium 34. The conveying roller 33 conveys the recording medium 34. The recording medium 34 is paper, for example. The fixing unit 35 fixes an image on the recording medium 34.

FIGS. 7B and 7C are schematic views of the exposure light source 28 in which a plurality of light-emitting portions 36 are arranged on a long substrate. An arrow 37 indicates a longitudinal direction in which the organic light-emitting devices are arranged. This column direction is the same as the direction of the rotation axis of the photosensitive member 27. This direction can also be referred to as the major-axis direction of the photosensitive member 27. In FIG. 7B, the light-emitting portions 36 are arranged in the major-axis direction of the photosensitive member 27. In FIG. 7C, unlike FIG. 7B, the light-emitting portions 36 are alternately arranged in the column direction in the first and second columns. The first and second columns are arranged at different positions in the row direction. In the first column, the light-emitting portions 36 are arranged at intervals. In the second column, the light-emitting portions 36 are arranged at positions corresponding to the spaces between the light-emitting portions 36 of the first column. Thus, the light-emitting portions 36 are also arranged at intervals in the row direction. The arrangement in FIG. 7C can also be referred to as a grid-like pattern, a staggered pattern, or a checkered pattern, for example.

As described above, an apparatus including the organic light-emitting device according to the present embodiment can be used to stably display a high-quality image for extended periods.

### EXEMPLARY EMBODIMENTS

The present invention is described below with exemplary embodiments. However, the present invention is not limited to these exemplary embodiments.

### Exemplary Embodiment 1 (Synthesis of Exemplary Compound A2)

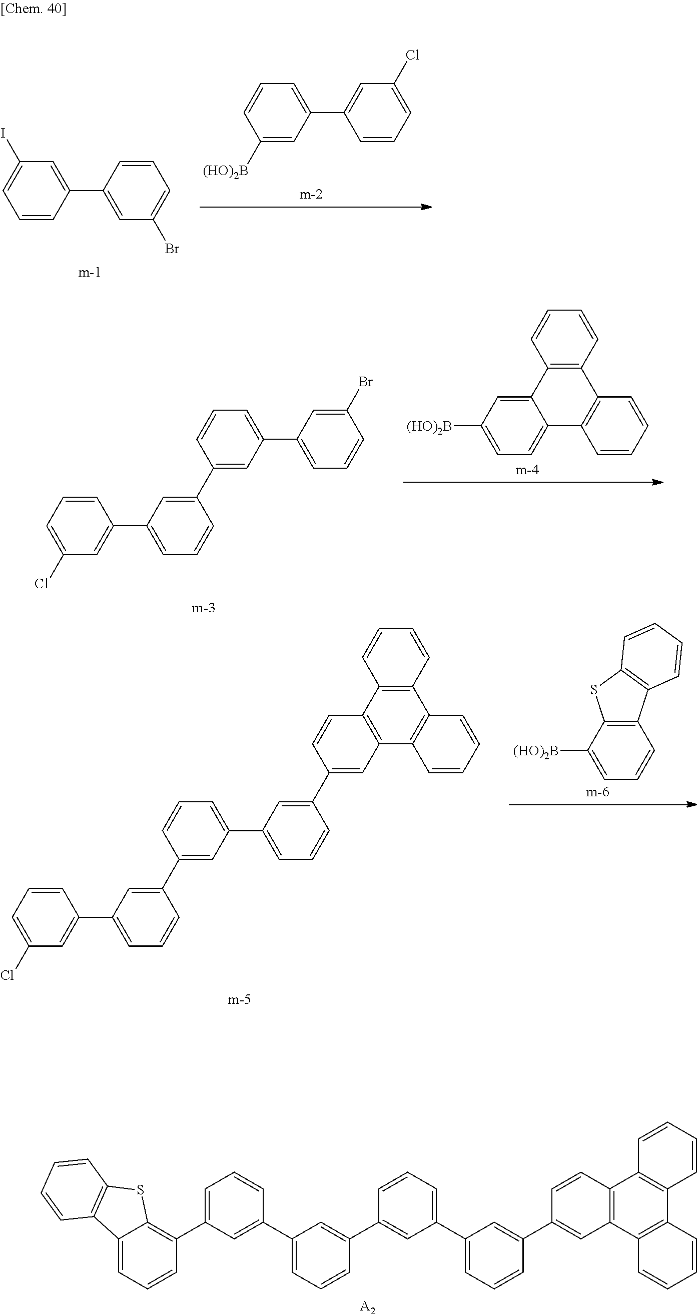

### (1) Synthesis of Compound m-3

A 200-ml recovery flask was charged with the following reagent(s) and solvent(s).
Compound m-1: 4.0 g (11.1 mmol)
Compound m-2: 1.7 g (11.1 mmol)
Pd(PPh₃) 4: 0.13 g
Toluene: 40 ml
Ethanol: 20 ml
Aqueous 2M sodium carbonate: 20 ml

The reaction solution was then heated and stirred under reflux in a nitrogen stream for 6 hours. After completion of the reaction, water was added to the product for separation. The product was dissolved in chloroform, was purified by column chromatography (chloroform:heptane), and was recrystallized in toluene/heptane. Thus, 3.0 g (yield: 78%) of a compound m-3 as a white solid was produced.

### (2) Synthesis of Compound m-5

A 200-ml recovery flask was charged with the following reagent(s) and solvent(s).
Compound m-3: 2.5 g (7.3 mmol)
Compound m-4: 1.8 g (8.0 mmol)
Pd(PPh₃)₄: 0.08 g
Toluene: 25 ml
Ethanol: 13 ml
Aqueous 2M sodium carbonate: 20 ml

The reaction solution was then heated and stirred under reflux in a nitrogen stream for 6 hours. After completion of the reaction, water was added to the product for separation. The product was dissolved in chloroform, was purified by column chromatography (chloroform:heptane), and was recrystallized in toluene/heptane. Thus, 4.0 g (yield: 82%) of a compound m-5 as a white solid was produced.

### (3) Synthesis of Compound A2

A 200-ml recovery flask was charged with the following reagent(s) and solvent(s).
Compound m-5: 2.0 g (4.5 mmol)
Compound m-6: 1.4 g (5.0 mmol)
Pd(dba)₂: 0.52 g
sphos: 0.74 g
Potassium phosphate: 2.88 g
Toluene: 100 ml
H₂O: 10 ml

The reaction solution was then heated and stirred under reflux in a nitrogen stream for 6 hours. After completion of the reaction, water was added to the product for separation. The product was dissolved in chloroform, was purified by column chromatography (chloroform:heptane), and was recrystallized in toluene/heptane. Thus, 5.2 g (yield: 74%) of Exemplary Compound A2 as a white solid was produced.

Exemplary Compound A2 was subjected to mass spectrometry with MALDI-TOF-MS (Autoflex LRF manufactured by Bruker).

[MALDI-TOF-MS] Measured value: m/z=714 Calculated value: C₅₄H₃₄S=71

### Exemplary Embodiments 2 to 20 (Synthesis of Exemplary Compounds)

In Tables 7-1 to 7-3, exemplary compounds of Exemplary Embodiments 2 to 20 were synthesized in the same manner as in Exemplary Embodiment 1 except that the raw materials m-4 and m-6 of Exemplary Embodiment 1 were changed. The measured values m/z measured by mass spectrometry in the same manner as in Exemplary Embodiment 1 are also shown.

TABLE 7-1

| Exemplary | Exemplary | Raw material | Raw material |
|---|---|---|---|
| Embodiment | Compound | m-2 | m-4 |
| 2 | A3 | 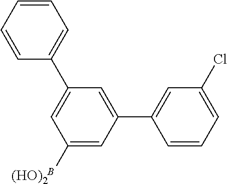 [SMILES: OB(O)c1cc(-c2ccccc2)cc(-c2cccc(Cl)c2)c1] | 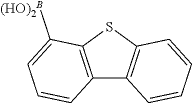 [SMILES: OB(O)c1cccc2c1sc1ccccc12] |
| 3 | A4 | 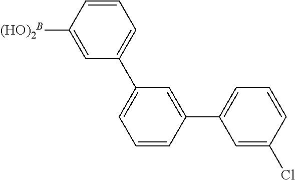 [SMILES: OB(O)c1cccc(-c2cccc(-c3cccc(Cl)c3)c2)c1] | 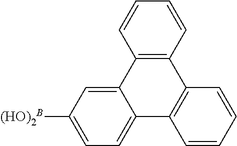 [SMILES: OB(O)c1ccc2c3ccccc3c3ccccc3c2c1] |
| 4 | A6 | 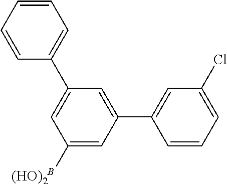 [SMILES: OB(O)c1cc(-c2ccccc2)cc(-c2cccc(Cl)c2)c1] | 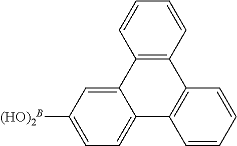 [SMILES: OB(O)c1ccc2c3ccccc3c3ccccc3c2c1] |
| 5 | A10 | 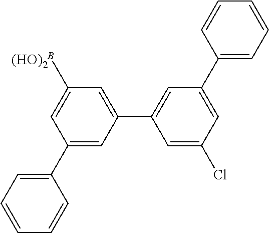 [SMILES: OB(O)c1cc(-c2ccccc2)cc(-c2cc(Cl)cc(-c3ccccc3)c2)c1] | 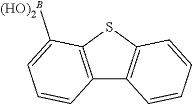 [SMILES: OB(O)c1cccc2c1sc1ccccc12] |
| 6 | A19 | 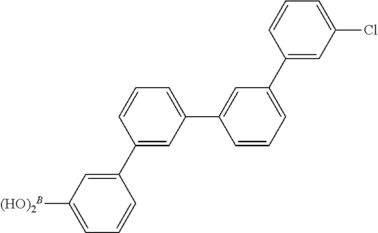 [SMILES: OB(O)c1cccc(-c2cccc(-c3cccc(-c4cccc(Cl)c4)c3)c2)c1] | 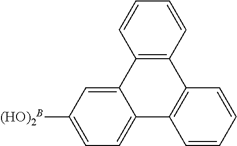 [SMILES: OB(O)c1ccc2c3ccccc3c3ccccc3c2c1] |
| 7 | A23 | 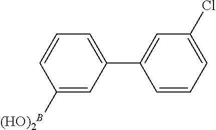 [SMILES: OB(O)c1cccc(-c2cccc(Cl)c2)c1] | 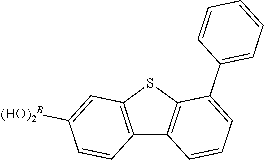 [SMILES: OB(O)c1ccc2c(c1)sc1c(-c3ccccc3)cccc12] |
| 8 | A28 | 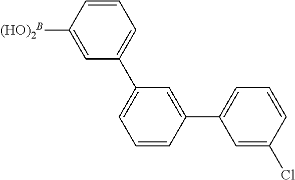 [SMILES: OB(O)c1cccc(-c2cccc(-c3cccc(Cl)c3)c2)c1] | 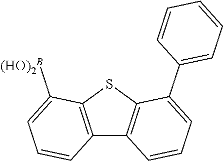 [SMILES: OB(O)c1cccc2c1sc1c(-c3ccccc3)cccc12] |
| 9 | A34 | 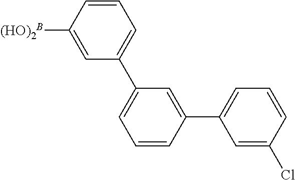 [SMILES: OB(O)c1cccc(-c2cccc(-c3cccc(Cl)c3)c2)c1] | 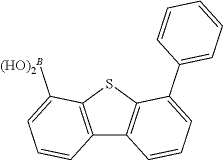 [SMILES: OB(O)c1cccc2c1sc1c(-c3ccccc3)cccc12] |

| Exemplary | Raw material | |
|---|---|---|
| Embodiment | m-6 | m/z |
| 2 | 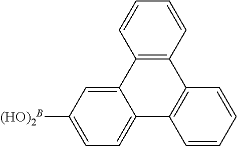 [SMILES: OB(O)c1ccc2c3ccccc3c3ccccc3c2c1] | 791 |
| 3 | 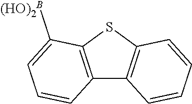 [SMILES: OB(O)c1cccc2c1sc1ccccc12] | 791 |
| 4 | 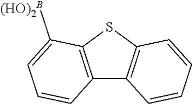 [SMILES: OB(O)c1cccc2c1sc1ccccc12] | 791 |
| 5 | 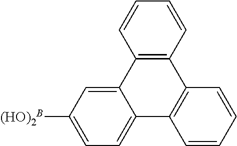 [SMILES: OB(O)c1ccc2c3ccccc3c3ccccc3c2c1] | 867 |
| 6 | 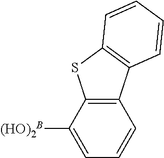 [SMILES: OB(O)c1cccc2c1sc1ccccc12] | 867 |
| 7 | 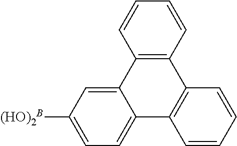 [SMILES: OB(O)c1ccc2c3ccccc3c3ccccc3c2c1] | 791 |
| 8 | 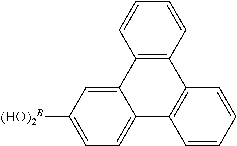 [SMILES: OB(O)c1ccc2c3ccccc3c3ccccc3c2c1] | 867 |
| 9 | 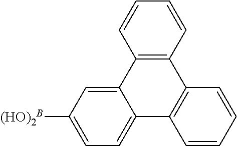 [SMILES: OB(O)c1ccc2c3ccccc3c3ccccc3c2c1] | 851 |

TABLE 7-2

| | | | |
|---|---|---|---|
| 10 | B4 | 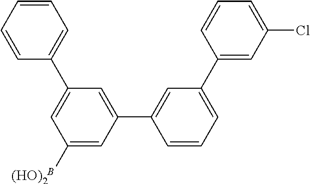 [SMILES: OB(O)c1cc(-c2ccccc2)cc(-c2cccc(-c3cccc(Cl)c3)c2)c1] | 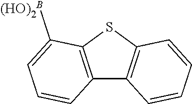 [SMILES: OB(O)c1cccc2c1sc1ccccc12] |
| 11 | B7 | 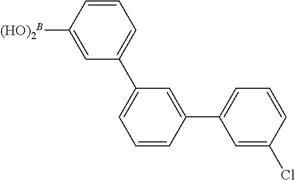 [SMILES: OB(O)c1cccc(-c2cccc(-c3cccc(Cl)c3)c2)c1] | 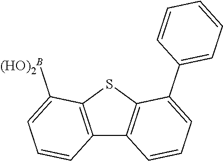 [SMILES: OB(O)c1cccc2c1sc1c(-c3ccccc3)cccc12] |
| 12 | B19 | 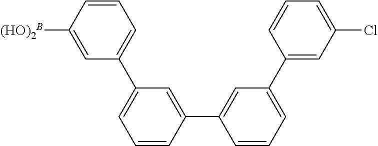 [SMILES: OB(O)c1cccc(-c2cccc(-c3cccc(-c4cccc(Cl)c4)c3)c2)c1] | 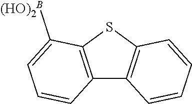 [SMILES: OB(O)c1cccc2c1sc1ccccc12] |
| 13 | B38 | 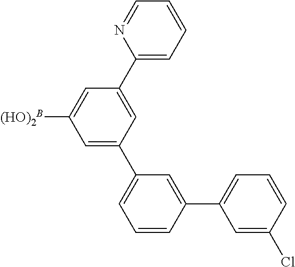 [SMILES: OB(O)c1cc(-c2cccc(-c3cccc(Cl)c3)c2)cc(-c2ccccn2)c1] | 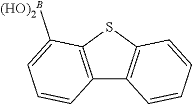 [SMILES: OB(O)c1cccc2c1sc1ccccc12] |
| 14 | C1 | 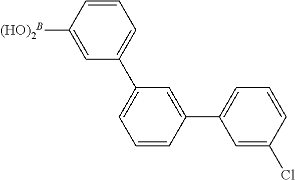 [SMILES: OB(O)c1cccc(-c2cccc(-c3cccc(Cl)c3)c2)c1] | 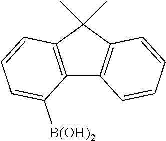 [SMILES: CC1(C)c2ccccc2-c2c(B(O)O)cccc21] |
| 15 | C2 | 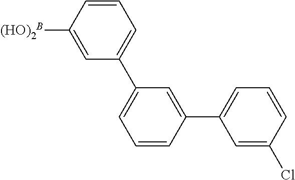 [SMILES: OB(O)c1cccc(-c2cccc(-c3cccc(Cl)c3)c2)c1] | 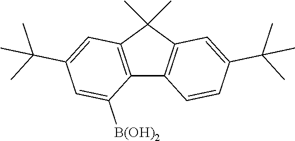 [SMILES: CC(C)(C)c1ccc2c(c1)C(C)(C)c1cc(C(C)(C)C)cc(B(O)O)c1-2] |
| 16 | C4 | 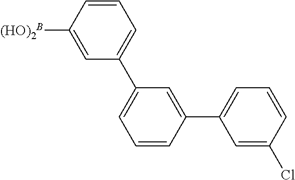 [SMILES: OB(O)c1cccc(-c2cccc(-c3cccc(Cl)c3)c2)c1] | 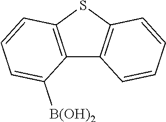 [SMILES: OB(O)c1cccc2sc3ccccc3c12] |
| 17 | C11 | 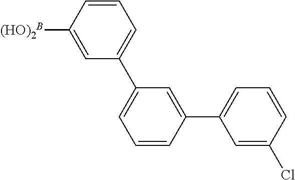 [SMILES: OB(O)c1cccc(-c2cccc(-c3cccc(Cl)c3)c2)c1] | 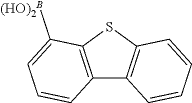 [SMILES: OB(O)c1cccc2c1sc1ccccc12] |
| 18 | D1 | 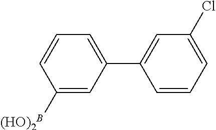 [SMILES: OB(O)c1cccc(-c2cccc(Cl)c2)c1] | 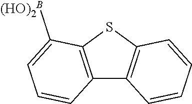 [SMILES: OB(O)c1cccc2c1sc1ccccc12] |

| | | |
|---|---|---|
| 10 | 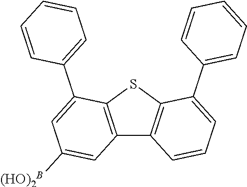 [SMILES: OB(O)c1cc(-c2ccccc2)c2sc3c(-c4ccccc4)cccc3c2c1] | 959 |
| 11 | 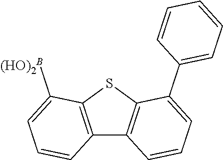 [SMILES: OB(O)c1cccc2c1sc1c(-c3ccccc3)cccc12] | 883 |
| 12 | 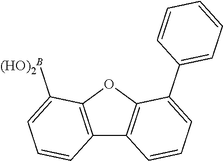 [SMILES: OB(O)c1cccc2c1oc1c(-c3ccccc3)cccc12] | 959 |
| 13 | 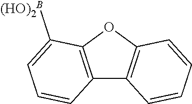 [SMILES: OB(O)c1cccc2c1oc1ccccc12] | 808 |
| 14 | 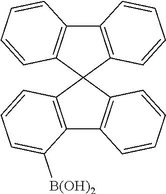 [SMILES: OB(O)c1cccc2c1-c1ccccc1C21c2ccccc2-c2ccccc21] | 889 |
| 15 | 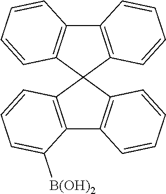 [SMILES: OB(O)c1cccc2c1-c1ccccc1C21c2ccccc2-c2ccccc21] | 1001 |
| 16 | 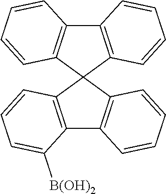 [SMILES: OB(O)c1cccc2c1-c1ccccc1C21c2ccccc2-c2ccccc21] | 879 |
| 17 | 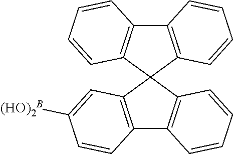 [SMILES: OB(O)c1ccc2c(c1)C1(c3ccccc3-c3ccccc31)c1ccccc1-2] | 881 |
| 18 | 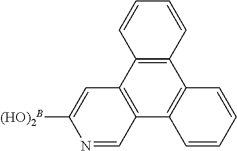 [SMILES: OB(O)c1cc2c3ccccc3c3ccccc3c2cn1] | 792 |

TABLE 7-3

| | | | | | |
|---|---|---|---|---|---|
| 19 | D19 | 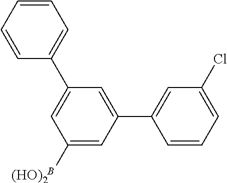 [SMILES: OB(O)c1cc(-c2ccccc2)cc(-c2cccc(Cl)c2)c1] | 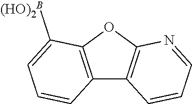 [SMILES: OB(O)c1cccc2c1oc1ncccc12] | 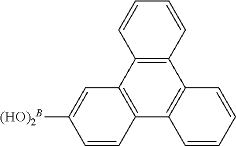 [SMILES: OB(O)c1ccc2c3ccccc3c3ccccc3c2c1] | 775 |
| 20 | D28 | 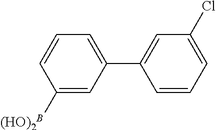 [SMILES: OB(O)c1cccc(-c2cccc(Cl)c2)c1] | 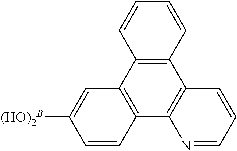 [SMILES: OB(O)c1ccc2c(c1)c1ccccc1c1cccnc12] | 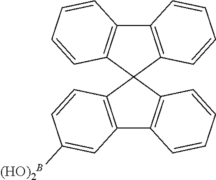 [SMILES: OB(O)c1ccc2c(c1)-c1ccccc1C21c2ccccc2-c2ccccc21] | 848 |

### Comparative Examples 1 to 4 (Synthesis of Comparative Compounds)

In Table 8, comparative compounds of Comparative Examples 1 to 3 were synthesized in the same manner as in Exemplary Embodiment 1 except that the raw materials m-1, m-2, m-4, and m-6 of Exemplary Embodiment 1 were changed. The measured values m/z measured by mass spectrometry in the same manner as in Exemplary Embodiment 1 are also shown.

TABLE 8

| Comparative | Comparative | | Raw material |
|---|---|---|---|
| Example | Compound | Raw material m-2 | m-4 |
| 1 | 1-B | 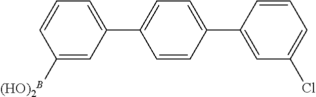 [SMILES: OB(O)c1cccc(-c2ccc(-c3cccc(Cl)c3)cc2)c1] | 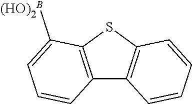 [SMILES: OB(O)c1cccc2c1sc1ccccc12] |
| 2 | 1-C | 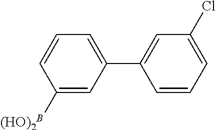 [SMILES: OB(O)c1cccc(-c2cccc(Cl)c2)c1] | 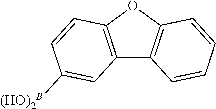 [SMILES: OB(O)c1ccc2oc3ccccc3c2c1] |
| 3 | 1-D | 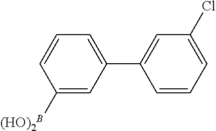 [SMILES: OB(O)c1cccc(-c2cccc(Cl)c2)c1] | 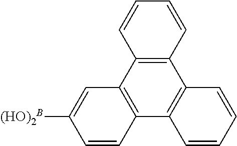 [SMILES: OB(O)c1ccc2c3ccccc3c3ccccc3c2c1] |

| Comparative | Raw material | |
|---|---|---|
| Example | m-6 | m/z |
| 1 | 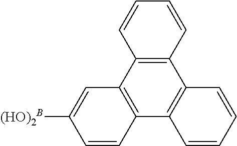 [SMILES: OB(O)c1ccc2c3ccccc3c3ccccc3c2c1] | 791 |
| 2 | 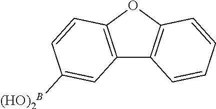 [SMILES: OB(O)c1ccc2oc3ccccc3c2c1] | 638 |
| 3 | 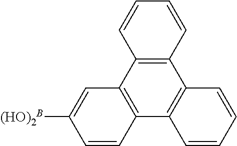 [SMILES: OB(O)c1ccc2c3ccccc3c3ccccc3c2c1] | 759 |

Comparative Compound 1-A was synthesized in accordance with the following scheme. Mass spectrometry was performed in the same manner as in Exemplary Embodiment 1.

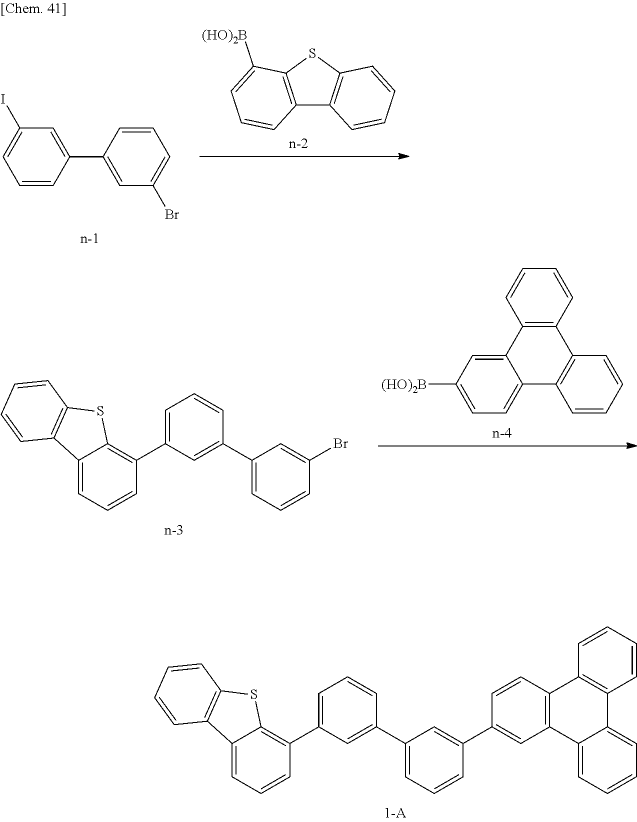 [SMILES: Brc1cccc(-c2cccc(I)c2)c1 ; OB(O)c1cccc2c1sc1ccccc12 ; Brc1cccc(-c2cccc(-c3cccc4c3sc3ccccc34)c2)c1 ; OB(O)c1ccc2c3ccccc3c3ccccc3c2c1 ; c1cc(-c2cccc(-c3cccc4c3sc3ccccc34)c2)cc(-c2ccc3c4ccccc4c4ccccc4c3c2)c1]

[MALDI-TOF-MS] Measured value: m/z=562 Calculated value: C₄₂H₂₆S=56

### Exemplary Embodiment 21

An organic light-emitting device of a bottom emission type was produced. The organic light-emitting device included a positive electrode, a hole injection layer, a hole transport layer, an electron-blocking layer, a light-emitting layer, a hole-blocking layer, an electron transport layer, an electron injection layer, and a negative electrode sequentially formed on a substrate.

First, an ITO film was formed on a glass substrate and was subjected to desired patterning to form an ITO electrode (positive electrode). The ITO electrode had a thickness of 100 nm. The substrate on which the ITO electrode was formed was used as an ITO substrate in the following process. Vacuum deposition was then performed by resistance heating in a vacuum chamber at 1.33×10⁻⁴ Pa to continuously form an organic compound layer and an electrode layer shown in Table 11 on the ITO substrate. The counter electrode (a metal electrode layer, a negative electrode) had an electrode area of 3 mm².

TABLE 9

| | | | | Film |
|---|---|---|---|---|
| | | | | thickness |
| | Material | | | (nm) |
| Negative electrode | Al | | | 100 |
| Electron injection layer (EIL) | LiF | | | 1 |
| Electron transport layer (ETL) | ET2 | | | 20 |
| Hole-blocking layer (HBL) | ET11 | | | 20 |
| Light-emitting layer (EML) | Host | A4 | Weight ratio | 20 |
| | Guest | AA11 | A4:AA11 = 90:10 | |

| | | |
|---|---|---|
| Electron-blocking layer (EBL) | HT19 | 15 |
| Hole transport layer (HTL) | HT3 | 30 |
| Hole injection layer (HIL) | HT16 | 5 |

Characteristics of the organic light-emitting device were measured and evaluated. The organic light-emitting device had a maximum emission wavelength of 522 nm and a maximum external quantum efficiency (E.Q.E.) of 13%.

A continuous operation test was performed at a current density of 100 mA/cm² to measure the time when the luminance decay rate reached 5%. The present exemplary embodiment had a luminance decay ratio of 1.4 on the assumption that the time when the luminance decay rate of Comparative Example 1 reached 5% was 1.0.

In the present exemplary embodiment, with respect to measuring apparatuses, more specifically, the current-voltage characteristics were measured with a microammeter 4140B manufactured by Hewlett-Packard Co., and the luminous brightness was measured with BM7 manufactured by Topcon Corporation.

### Exemplary Embodiments 22 to 41, Comparative Examples 5 to 8

In Exemplary Embodiments 22 to 41, organic light-emitting devices were produced in the same manner as in Exemplary Embodiment 21 except that the compounds shown in Tables 10-1 and 10-2 were appropriately used. Characteristics of the organic light-emitting device were measured and evaluated in the same manner as in Exemplary Embodiment 21. Table 10 shows the measurement results.

TABLE 10-1

| | | | | EML | | | | E.Q.E | Luminance |
|---|---|---|---|---|---|---|---|---|---|
| | HIL | HTL | EBL | Host | Guest | HBL | ETL | [%] | decay ratio |
| Exemplary Embodiment 22 | HT16 | HT3 | HT19 | A1 | AA26 | ET12 | ET15 | 13 | 1.3 |
| Exemplary Embodiment 23 | HT16 | HT2 | HT15 | A4 | AA27 | ET12 | ET2 | 14 | 1.4 |
| Exemplary Embodiment 24 | HT16 | HT2 | HT15 | A4 | AA30 | ET11 | ET2 | 13 | 1.4 |
| Exemplary Embodiment 25 | HT16 | HT3 | HT19 | A3 | CC24 | ET11 | ET2 | 13 | 1.3 |
| Exemplary Embodiment 26 | HT16 | HT3 | HT19 | A14 | GD10 | ET11 | ET2 | 10 | 1.2 |
| Exemplary Embodiment 27 | HT16 | HT3 | HT19 | A15 | HH1 | ET11 | ET15 | 12 | 1.3 |
| Exemplary Embodiment 28 | HT16 | HT3 | HT19 | B4 | BB24 | ET12 | ET2 | 13 | 1.1 |
| Exemplary Embodiment 29 | HT16 | HT2 | HT15 | B3 | BB25 | ET12 | ET15 | 13 | 1.1 |
| Exemplary Embodiment 30 | HT16 | HT3 | HT19 | B9 | GD10 | ET12 | ET15 | 11 | 1.2 |
| Exemplary Embodiment 31 | HT16 | HT2 | HT15 | B10 | DD5 | ET11 | ET2 | 13 | 1.2 |
| Exemplary Embodiment 32 | HT16 | HT3 | HT19 | B12 | DD31 | ET12 | ET15 | 14 | 1.2 |
| Exemplary Embodiment 33 | HT16 | HT2 | HT15 | C1 | DD17 | ET12 | ET2 | 14 | 1.1 |
| Exemplary Embodiment 34 | HT16 | HT2 | HT15 | C2 | EE1 | ET11 | ET2 | 13 | 1.1 |
| Exemplary Embodiment 35 | HT16 | HT3 | HT19 | C3 | EE2 | ET12 | ET15 | 13 | 1.1 |
| Exemplary Embodiment 36 | HT16 | HT3 | HT19 | C14 | FF29 | ET12 | ET15 | 12 | 1.1 |
| Exemplary Embodiment 37 | HT16 | HT3 | HT19 | C15 | HH21 | ET11 | ET15 | 13 | 1.2 |
| Exemplary Embodiment 38 | HT16 | HT3 | HT19 | D1 | FF1 | ET12 | ET15 | 12 | 1.2 |
| Exemplary Embodiment 39 | HT16 | HT3 | HT19 | D2 | GG4 | ET11 | ET15 | 13 | 1.2 |
| Exemplary Embodiment 40 | HT16 | HT3 | HT19 | D17 | HH27 | ET12 | ET2 | 13 | 1.2 |
| Exemplary Embodiment 41 | HT16 | HT2 | HT15 | D19 | 112 | ET12 | ET15 | 13 | 1.2 |
| Comparative Example 5 | HT16 | HT3 | HT19 | Comparative | GD10 | ET11 | ET2 | 10 | 1.0 |
| | | | | Compound 1-A | | | | | |

TABLE 10-2

| | | | | EML | | | | E.Q.E | Luminance |
|---|---|---|---|---|---|---|---|---|---|
| | HIL | HTL | EBL | Host | Guest | HBL | ETL | [%] | decay ratio |
| Comparative | HT16 | HT3 | HT19 | Comparative | GD10 | ET11 | ET2 | 8 | 0.7 |
| Example 6 | | | | Compound | | | | | |
| | | | | 1-B | | | | | |
| Comparative | HT16 | HT3 | HT19 | Comparative | GD10 | ET11 | ET2 | 10 | 0.7 |
| Example 7 | | | | Compound | | | | | |
| | | | | 1-C | | | | | |
| Comparative | HT16 | HT3 | HT19 | Comparative | GD10 | ET11 | ET2 | 8 | 0.9 |
| Example 8 | | | | Compound | | | | | |
| | | | | 1-D | | | | | |

Tables 10-1 and 10-2 show that Comparative Examples 5 to 8 had an E.Q.E. of 10%, 8%, 10%, and 8%, respectively. Comparative Examples 5 to 8 had a luminance decay ratio of 1.0, 0.7, 0.7, and 0.9, respectively. This is probably because Comparative Compound 1-A has a low Tg and low film stability. This is probably because Comparative Compound 1-B has low T1 energy and long excitation lifetime. This is probably because Comparative Compound 1-C has a low Tg and low film stability. This is probably because Comparative Compound 1-D has low T1 energy and low sublimability. The term “film stability” refers to the resistance of the film quality to change during the operation of an organic light-emitting device. Thus, “low film stability” means that the film quality is likely to change during the operation of an organic light-emitting device.

On the other hand, an organic light-emitting device according to the present invention had high light emission efficiency and long device life. This is because the exemplary compounds according to the present invention have high T1 energy and high Tg.

Furthermore, an organic light-emitting device particularly with high light emission efficiency and long device life could be produced by selecting, as a ligand, a light-emitting material with a tricyclic or polycyclic fused ring suitable for combination with an organic compound according to the present invention.

Thus, an organic compound according to the present invention can be used to provide an organic light-emitting device with high light emission efficiency and long device life.

### Exemplary Embodiment 42

An organic light-emitting device was produced in the same manner as in Exemplary Embodiment 21 except that the organic compound layer and the electrode layer shown in Table 11 were continuously formed.

TABLE 11

| | | | | Film |
|---|---|---|---|---|
| | | | | thickness |
| | Material | | | (nm) |
| Negative electrode | Al | | | 100 |
| Electron injection layer (EIL) | LiF | | | 1 |
| Electron transport layer | ET2 | | | 20 |
| (ETL) | | | | |
| Hole-blocking layer (HBL) | ET11 | | | 20 |
| Light-emitting layer (EML) | Host | A4 | Weight ratio | 20 |
| | Guest | AA25 | A4:AA25:EM30 = | |
| | Assist | EM30 | 60:10:30 | |

| | | |
|---|---|---|
| Electron-blocking layer (EBL) | HT19 | 15 |
| Hole transport layer (HTL) | HT3 | 30 |
| Hole injection layer (HIL) | HT16 | 5 |

Characteristics of the organic light-emitting device were measured and evaluated. The organic light-emitting device had a green emission color and had an E.Q.E. of 19%.

### Exemplary Embodiments 43 to 67

In Exemplary Embodiments 43 to 67, organic light-emitting devices were produced in the same manner as in Exemplary Embodiment 42 except that the compounds shown in Tables 12-1 and 12-2 were appropriately used.

Characteristics of the organic light-emitting devices were measured and evaluated in the same manner as in Exemplary Embodiment 42. Table 12 shows the measurement results.

TABLE 12-1

| | | | | EML | | | | | E.Q.E |
|---|---|---|---|---|---|---|---|---|---|
| | HIL | HTL | EBL | Host | Guest | Assist | HBL | ETL | [%] |
| Exemplary Embodiment 43 | HT16 | HT3 | HT19 | B19 | BB24 | EM29 | ET26 | ET3 | 17 |
| Exemplary Embodiment 44 | HT16 | HT2 | HT15 | B18 | BB21 | EM35 | ET13 | ET2 | 16 |
| Exemplary Embodiment 45 | HT16 | HT2 | HT15 | C11 | CC17 | EM37 | ET13 | ET2 | 15 |
| Exemplary Embodiment 46 | HT16 | HT3 | HT19 | A8 | DD5 | EM31 | ET16 | ET15 | 18 |
| Exemplary Embodiment 47 | HT16 | HT3 | HT19 | A7 | DD31 | EM30 | ET16 | ET15 | 18 |
| Exemplary Embodiment 48 | HT16 | HT3 | HT19 | B28 | DD27 | EM28 | ET17 | ET15 | 16 |
| Exemplary Embodiment 49 | HT16 | HT3 | HT19 | A21 | AA25 | EM39 | ET13 | ET2 | 18 |
| Exemplary Embodiment 50 | HT16 | HT2 | HT15 | C4 | EE2 | GD10 | ET15 | ET3 | 13 |

TABLE 12-2

| | | | | | | | | | |
|---|---|---|---|---|---|---|---|---|---|
| Exemplary | HT16 | HT3 | HT19 | A5 | AA15 | ET15 | ET15 | ET15 | 17 |
| Embodiment | | | | | | | | | |
| 51 | | | | | | | | | |
| Exemplary | HT16 | HT2 | HT15 | A4 | AA26 | ET16 | ET2 | ET2 | 18 |
| Embodiment | | | | | | | | | |
| 52 | | | | | | | | | |
| Exemplary | HT16 | HT3 | HT19 | D21 | FF1 | EM16 | ET26 | ET3 | 13 |
| Embodiment | | | | | | | | | |
| 53 | | | | | | | | | |
| Exemplary | HT16 | HT2 | HT15 | B6 | GG3 | EM16 | ET13 | ET2 | 14 |
| Embodiment | | | | | | | | | |
| 54 | | | | | | | | | |
| Exemplary | HT16 | HT2 | HT15 | B5 | HH25 | EM34 | ET13 | ET2 | 15 |
| Embodiment | | | | | | | | | |
| 55 | | | | | | | | | |
| Exemplary | HT16 | HT3 | HT19 | C18 | GG21 | EM35 | ET16 | ET15 | 14 |
| Embodiment | | | | | | | | | |
| 56 | | | | | | | | | |
| Exemplary | HT16 | HT3 | HT19 | A7 | DD46 | EM37 | ET16 | ET15 | 17 |
| Embodiment | | | | | | | | | |
| 57 | | | | | | | | | |
| Exemplary | HT16 | HT2 | HT15 | B4 | DD35 | EM37 | ET15 | ET3 | 15 |
| Embodiment | | | | | | | | | |
| 58 | | | | | | | | | |
| Exemplary | HT16 | HT3 | HT19 | B2 | DD31 | EM30 | ET15 | ET15 | 16 |
| Embodiment | | | | | | | | | |
| 59 | | | | | | | | | |
| Exemplary | HT16 | HT2 | HT15 | B3 | DD9 | EM28 | ET2 | ET2 | 16 |
| Embodiment | | | | | | | | | |
| 60 | | | | | | | | | |
| Exemplary | HT16 | HT3 | HT19 | B9 | DD8 | EM28 | ET26 | ET3 | 16 |
| Embodiment | | | | | | | | | |
| 61 | | | | | | | | | |
| Exemplary | HT16 | HT2 | HT15 | B5 | DD3 | EM29 | ET13 | ET2 | 15 |
| Embodiment | | | | | | | | | |
| 62 | | | | | | | | | |
| Exemplary | HT16 | HT3 | HT19 | D1 | AA6 | EM30 | ET26 | ET3 | 16 |
| Embodiment | | | | | | | | | |
| 63 | | | | | | | | | |
| Exemplary | HT16 | HT2 | HT15 | D18 | HH7 | AA6 | ET13 | ET2 | 17 |
| Embodiment | | | | | | | | | |
| 64 | | | | | | | | | |
| Exemplary | HT16 | HT2 | HT15 | C3 | HH25 | EM30 | ET13 | ET2 | 14 |
| Embodiment | | | | | | | | | |
| 65 | | | | | | | | | |
| Exemplary | HT16 | HT3 | HT19 | C2 | II2 | GD12 | D60 | ET15 | 14 |
| Embodiment | | | | | | | | | |
| 66 | | | | | | | | | |
| Exemplary | HT16 | HT3 | HT19 | B16 | II3 | GD10 | D54 | ET15 | 13 |
| Embodiment | | | | | | | | | |
| 67 | | | | | | | | | |

Tables 12-1 and 12-2 show that the use of an organic compound according to the present invention and the assist material suitable for the combination of the organic compound according to the present invention improved the light emission efficiency of the organic light-emitting device. The use of a compound with at least one of a carbazole skeleton, an azine ring, and a xanthone skeleton as the assist material improved the light emission efficiency of the organic light-emitting device. All of the organic light-emitting devices used in Exemplary Embodiments 42 to 67 had a high luminance decay ratio equal to or higher than that of Exemplary Embodiments 21 to 41.

As described above, an organic compound according to the present invention provides a film with high thermal stability and sublimability. Furthermore, molecules of the organic compound are less likely to aggregate. Thus, an organic compound according to the present invention can be used for an organic light-emitting device to provide the organic light-emitting device with good emission properties and long device life.

The present invention may have the following configurations:

### (Configuration 1)

An organic compound represented by the following general formula [1]:

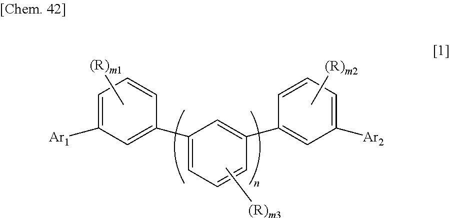

In the general formula [1], Ar₁ and Ar₂ are each independently selected from a substituted or unsubstituted tricyclic or polycyclic aryl group and a substituted or unsubstituted tricyclic or polycyclic heterocyclic group. Ar₁ and Ar₂ are represented by different skeletons. When Ar₁ and Ar₂ have a dibenzothiophene skeleton or a dibenzofuran skeleton, the organic compound has at least one substituent. A substituent represented by R is each independently selected from a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group. When a plurality of Rs are present, the Rs may be the same or different. n denotes an integer in the range of 2 to 5, and m₁ to m₃ each denote an integer in the range of 0 to 4.

### (Configuration 2)

The organic compound according to Configuration 1, wherein, in Ar₁ and Ar₂ in the general formula [1], Ar₁ denotes the aryl group, and Ar₂ denotes the aryl group or the heterocyclic group different from Ar₁.

### (Configuration 3)

The organic compound according to Configuration 2, wherein, in Ar₁ and Ar₂ in the general formula [1], Ar₁ denotes the aryl group, and Ar₂ denotes the heterocyclic group.

### (Configuration 4)

The organic compound according to Configuration 3, wherein, in Ar₁ and Ar₂ in the general formula [1], Ar₁ is selected from a substituent A group, and Ar₂ is selected from a substituent B group.

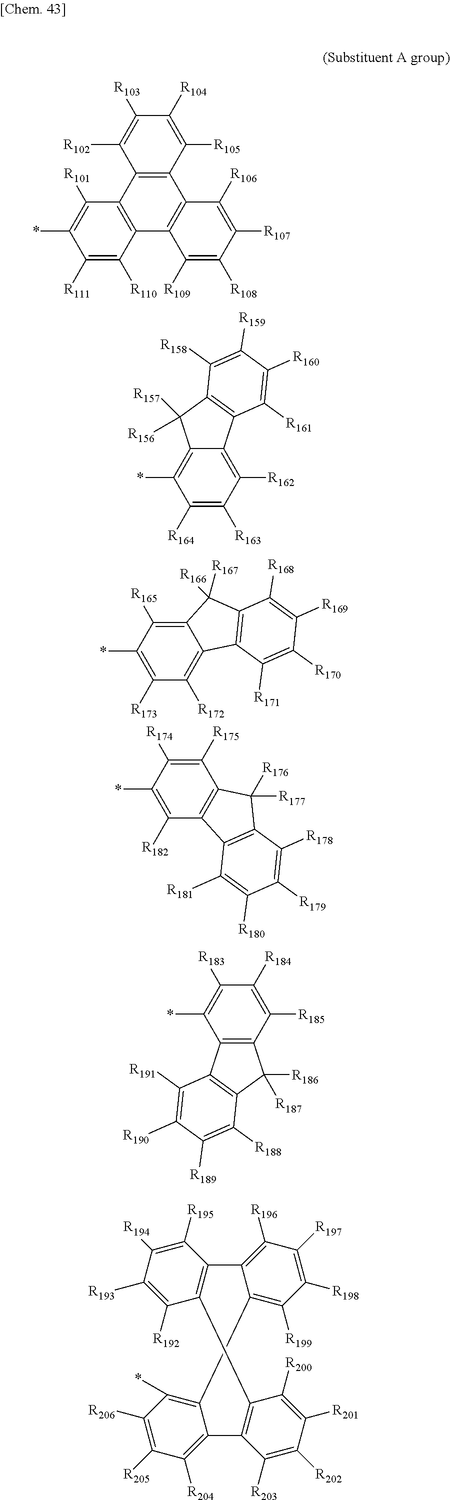

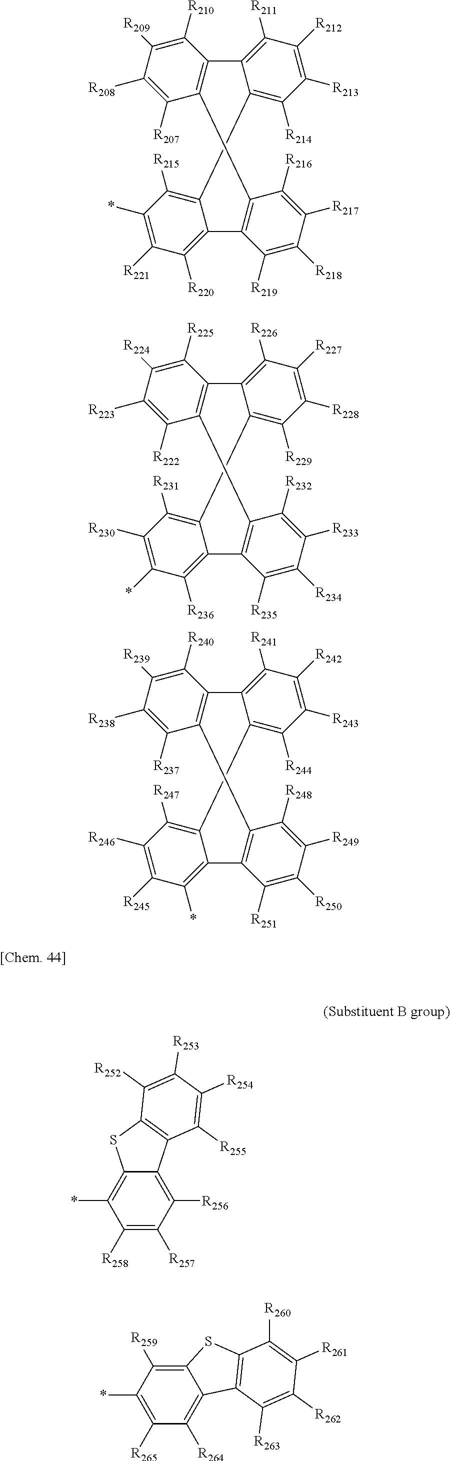

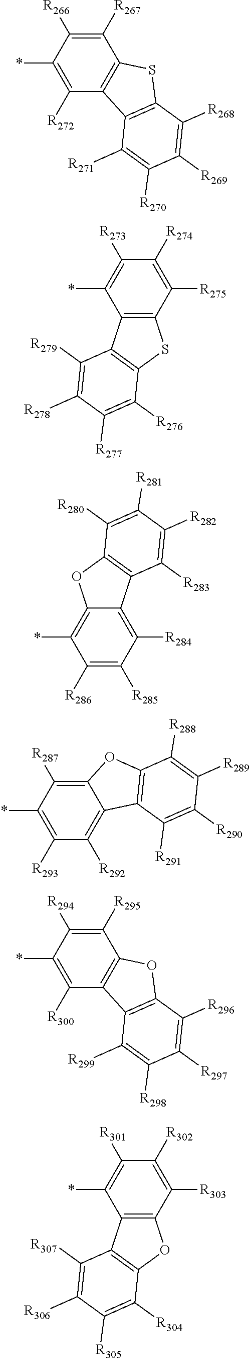

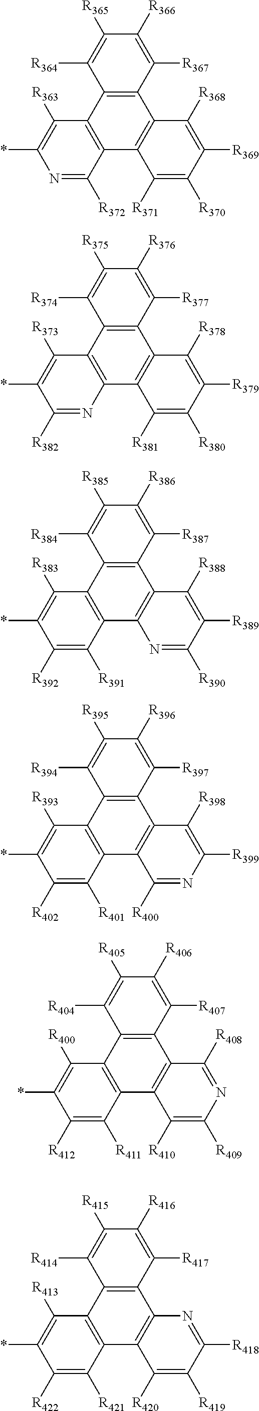

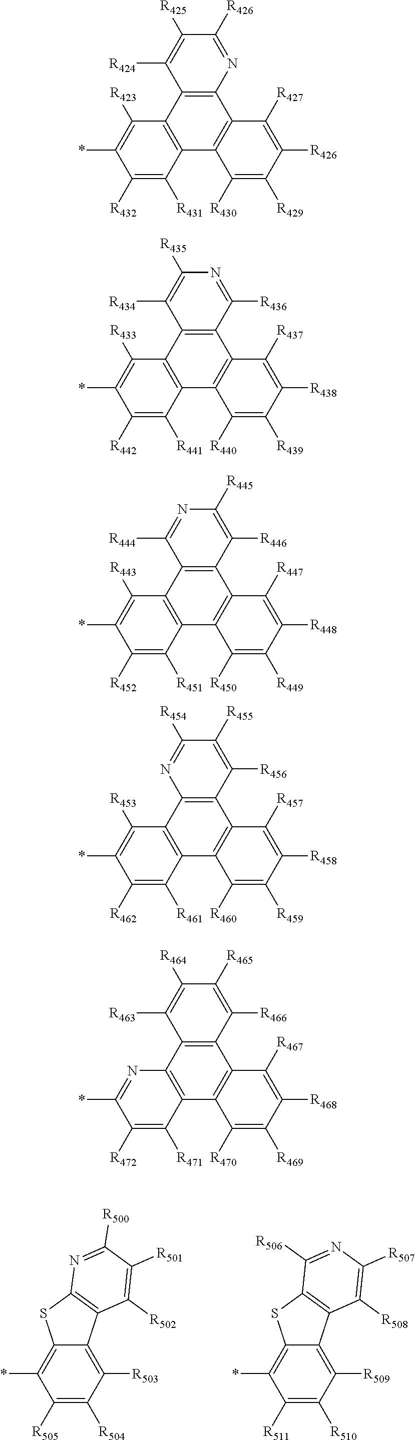

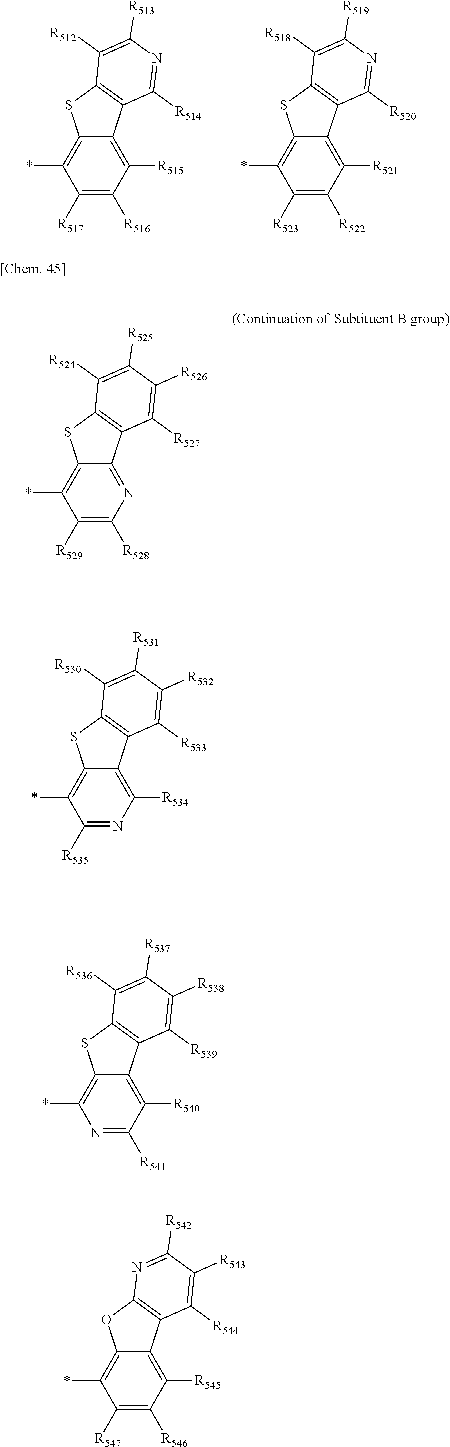

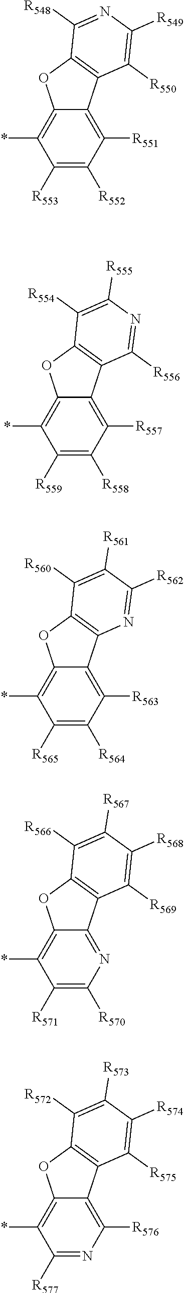

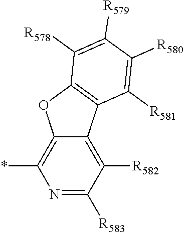

R₁₀₁ to R₅₈₃ in the substituent A group and the substituent B group are each independently selected from a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group. * represents a binding position to a phenylene group.

### (Configuration 5)

The organic compound according to Configuration 4, wherein, in Ar₁ and Ar₂ in the general formula [1], Ar₁ is selected from a substituent C group, and Ar₂ is selected from a substituent D group.

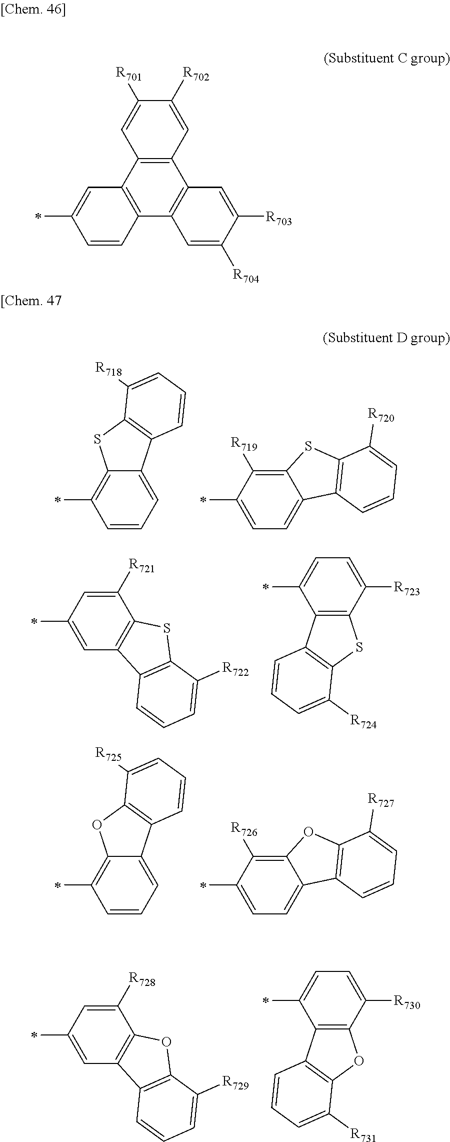

R₇₀₁ to R₇₃₁ in the substituent C group and the substituent D group are each independently selected from a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group. * represents a binding position to a phenylene group.

### (Configuration 6)

The organic compound according to Configuration 4 or 5, wherein R₁₀₁ to R₈₆₈ in the substituent A group to the substituent D group are selected from a hydrogen atom, a deuterium atom, an alkyl group with 1 to 4 carbon atoms, an aryl group with 6 to 18 carbon atoms, a heterocyclic group with 5 to 15 carbon atoms, a trimethylsilyl group, a triphenylsilyl group, and a cyano group.

### (Configuration 7)

The organic compound according to Configuration 6, wherein R₁₀₁ to R₈₆₈ in the substituent A group to the substituent D group are selected from a hydrogen atom, a phenyl group, and a tert-butyl group.

### (Configuration 8)

The organic compound according to any one of Configurations 1 to 7, wherein R in the general formula [1] denotes an aryl group with 6 to 18 carbon atoms or a heterocyclic group with 5 to 9 carbon atoms.

### (Configuration 9)

The organic compound according to Configuration 8, wherein R in the general formula [1] denotes a phenyl group or a pyridyl group.

### (Configuration 10)

The organic compound according to any one of Configurations 1 to 9, wherein n in the general formula [1] is 3 or 4.

### (Configuration 11)

The organic compound according to any one of Configurations 1 to 10, wherein m in the general formula [1] is 0.

### (Configuration 12)

An organic light-emitting device including: a first electrode;
a second electrode; and
an organic compound layer between the first electrode and the second electrode,
wherein the organic compound layer contains the organic compound according to any one of Configurations 1 to 11.

### (Configuration 13)

The organic light-emitting device according to Configuration 12, wherein the organic compound layer includes a light-emitting layer, and the light-emitting layer contains the organic compound.

### (Configuration 14)

The organic light-emitting device according to Configuration 13, wherein the light-emitting layer further contains a first compound, and the organic compound has a lowest excited singlet energy higher than a lowest excited triplet energy of the first compound.

### (Configuration 15)

The organic light-emitting device according to Configuration 14, wherein the first compound has at least a tricyclic or polycyclic fused-ring structure.

### (Configuration 16)

The organic light-emitting device according to Configuration 15, wherein the first compound has a structure represented by one of the general formulae [Ir-1] to [Ir-12].

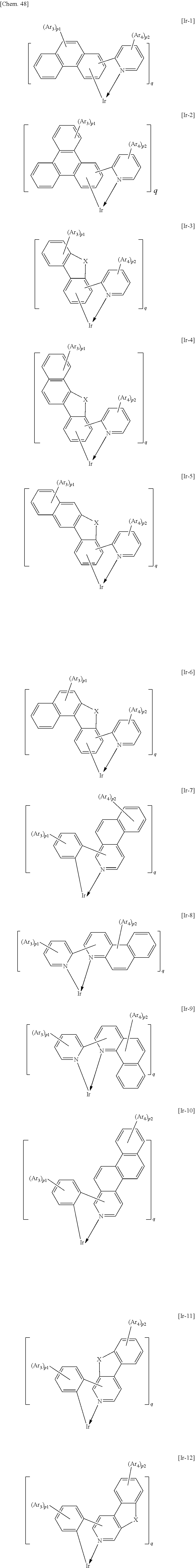

In the general formulae [Ir-1] to [Ir-12], Ar₃ and Ar₄ denote a deuterium atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted silyl group, or a cyano group. X is selected from an oxygen atom, a sulfur atom, C(R₁) (R₂), and NR₃. R₁ to R₃ are each independently selected from a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group. p₁ and p₂ each denote an integer in the range of 0 to 4, and q denotes an integer in the range of 1 to 3.

### (Configuration 17)

The organic light-emitting device according to Configuration 16, wherein the first compound has any one of a triphenylene skeleton, a phenanthrene skeleton, a fluorene skeleton, a benzofluorene skeleton, a dibenzofuran skeleton, a dibenzothiophene skeleton, a benzoisoquinoline skeleton, and a naphthoisoquinoline skeleton.

### (Configuration 18)

The organic light-emitting device according to any one of Configurations 14 to 17, wherein the light-emitting layer further contains a second compound, and the second compound has a lowest excited singlet energy higher than a lowest excited singlet energy of the first compound.

### (Configuration 19)

The organic light-emitting device according to Configuration 18, wherein the second compound has at least one of a carbazole skeleton, an azine ring, and a xanthone skeleton.

### (Configuration 20)

A display apparatus including a plurality of pixels, wherein at least one of the plurality of pixels includes the organic light-emitting device according to any one of Configurations 12 to 19 and a transistor coupled to the organic light-emitting device.

### (Configuration 21)

A photoelectric conversion apparatus including: an optical unit including a plurality of lenses; an imaging device configured to receive light passing through the optical unit; and a display unit configured to display an image taken by the imaging device, wherein the display unit includes the organic light-emitting device according to any one of Configurations 12 to 19.

### (Configuration 22)

Electronic equipment including: a display unit including the organic light-emitting device according to any one of Configurations 12 to 19; a housing configured to be provided with the display unit; and a communication unit provided in the housing and configured to communicate with an outside.

### (Configuration 23)

A lighting apparatus including: a light source including the organic light-emitting device according to any one of Configurations 12 to 19; and a light-diffusing unit or an optical film configured to transmit light emitted by the light source.

### (Configuration 24)

A moving body including: a lamp including the organic light-emitting device according to any one of Configurations 12 to 19; and a body configured to be provided with the lamp.

### (Configuration 25)

An image-forming apparatus including: a photosensitive member; and an exposure light source configured to expose the photosensitive member to light,
wherein the exposure light source includes the organic light-emitting device according to any one of Configurations 12 to 19.

The present invention is not limited to these embodiments, and various changes and modifications may be made therein without departing from the spirit and scope of the present invention. Accordingly, the following claims are attached to make the scope of the present invention public.

According to the present invention, an organic compound according to the present invention can be used for an organic light-emitting device to provide the organic light-emitting device with long device life.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An organic compound represented by the following general formula [1]: 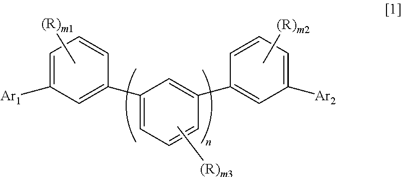
wherein, in Ar₁ and Ar₂ in the general formula [1], Ar₁ is selected from a substituent A group, and Ar₂ is selected from a substituent B group, 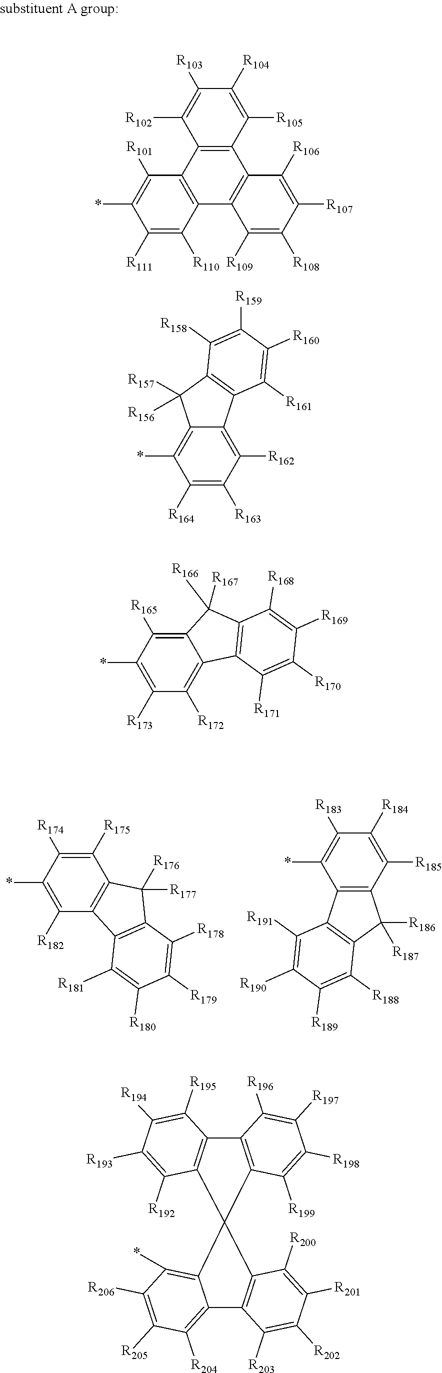 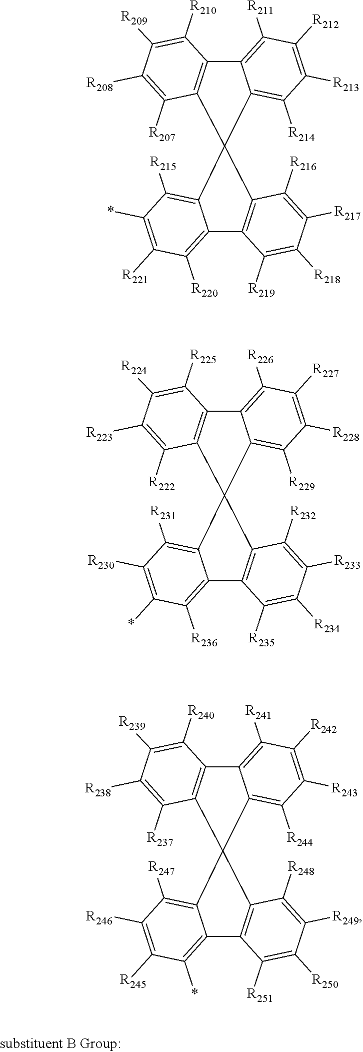 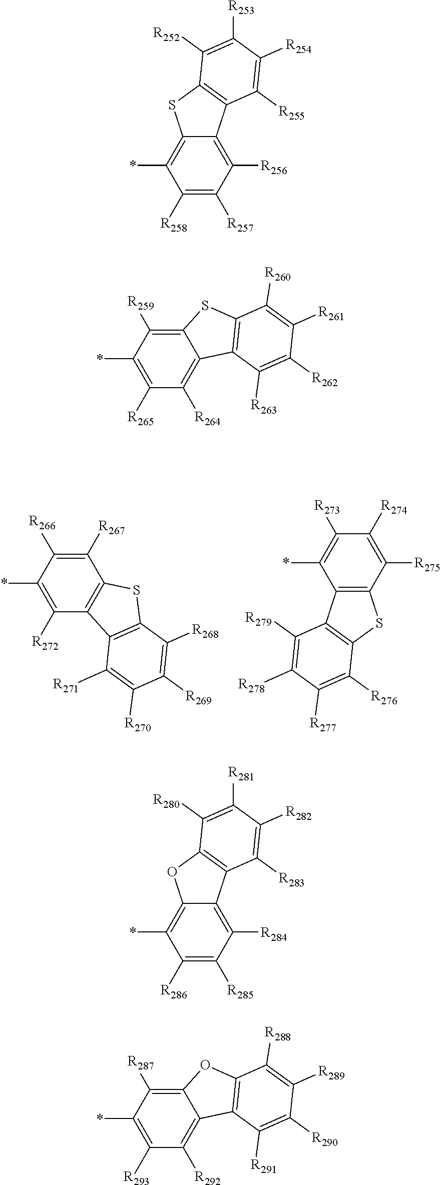 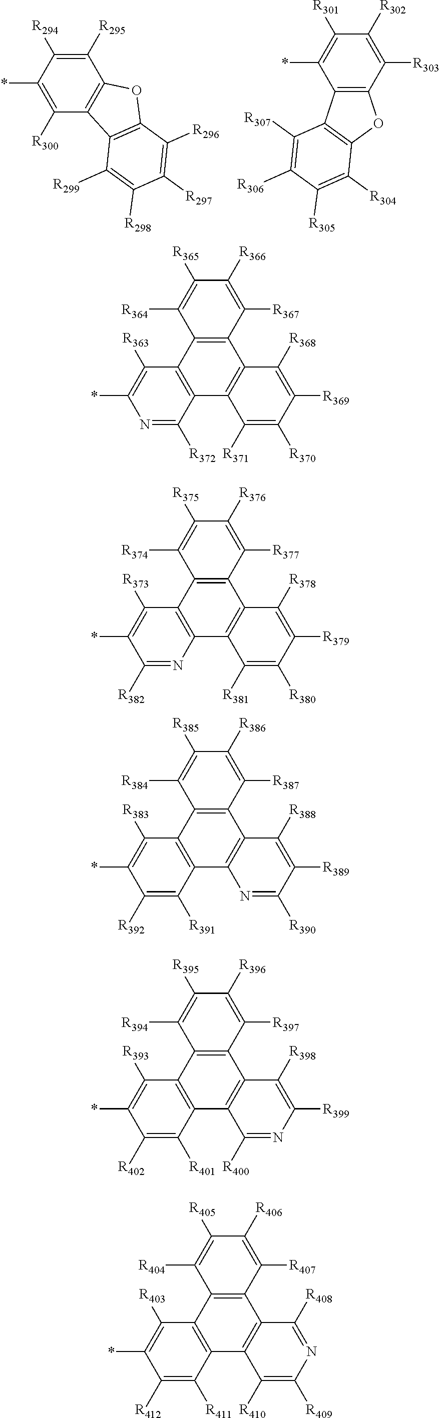 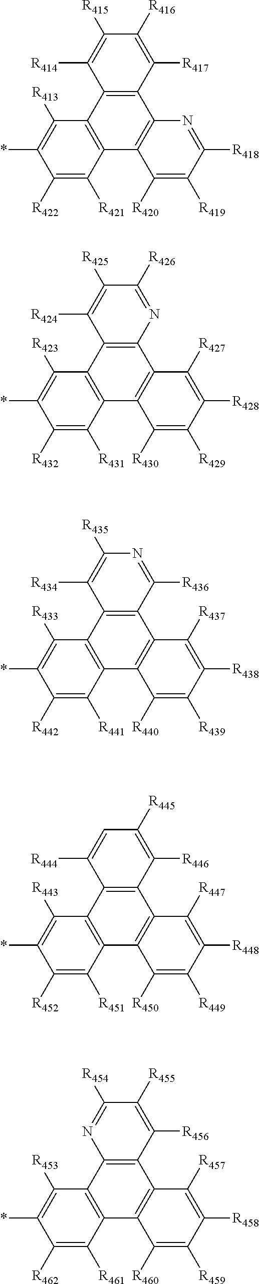 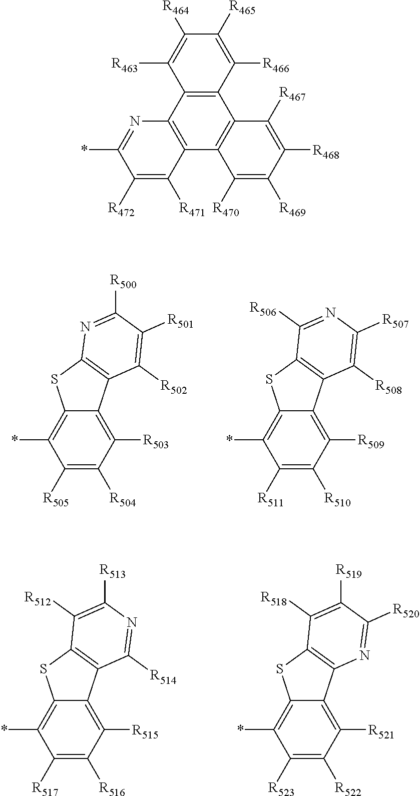 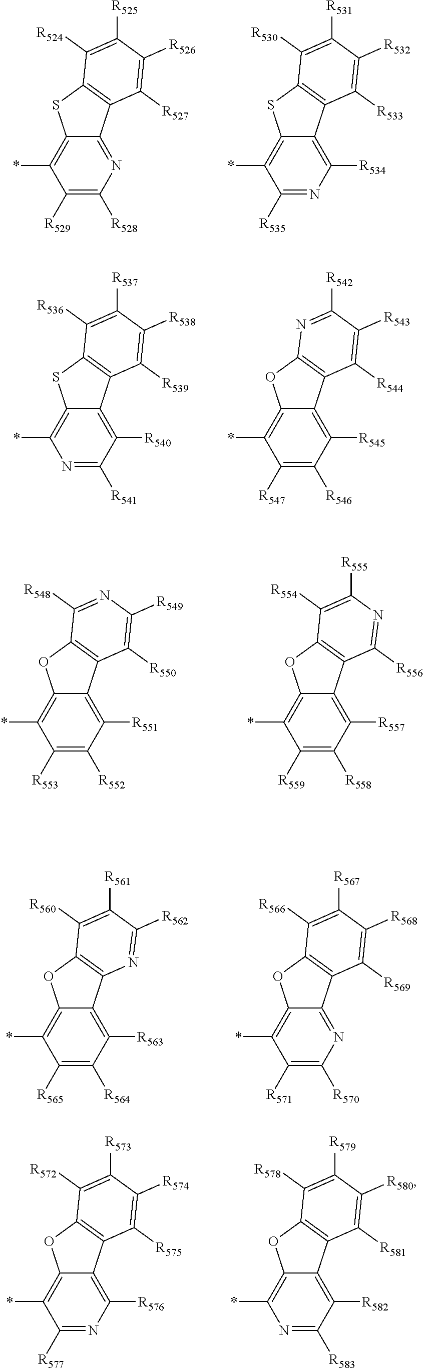
wherein R₁₀₁ to R₅₈₃ in the substituent A group and the substituent B group are each independently selected from a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group, and * represents a binding position to a phenylene group a substituent represented by R is each independently selected from a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group, n denotes an integer in the range of 2 to 5, and m₁ to m₃ each denote 0.

2. The organic compound according to claim 1, wherein, in Ar₁ and Ar₂ in the general formula [1], Ar₁ is selected from a substituent C group, and Ar₂ is selected from a substituent D group, 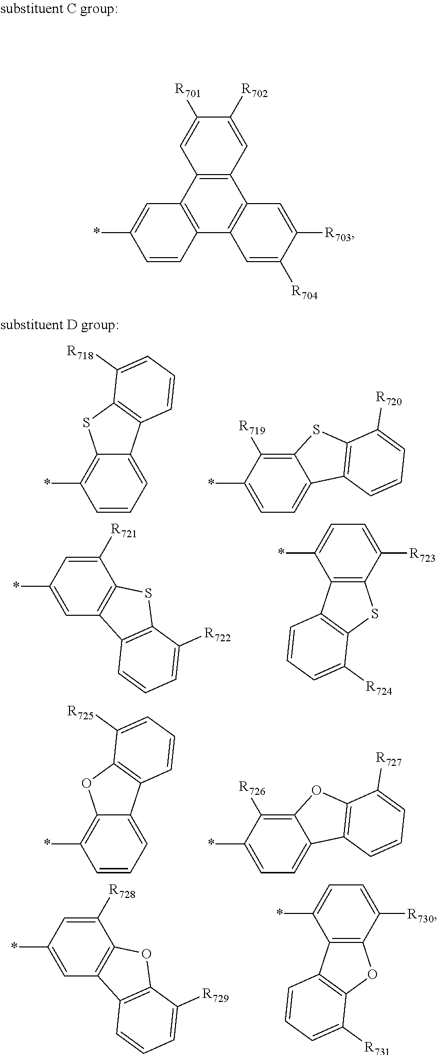
wherein R₇₀₁ to R₇₃₁ in the substituent C group and the substituent D group are each independently selected from a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group, and * represents a binding position to a phenylene group.

3. The organic compound according to claim 1, wherein R₁₀₁ to R₈₆₈ in the substituent A group to the substituent D group are selected from a hydrogen atom, a deuterium atom, an alkyl group with 1 to 4 carbon atoms, an aryl group with 6 to 18 carbon atoms, a heterocyclic group with 5 to 15 carbon atoms, a trimethylsilyl group, a triphenylsilyl group, and a cyano group.

4. The organic compound according to claim 1, wherein R₁₀₁ to R₈₆₈ in the substituent A group to the substituent D group are selected from a hydrogen atom, a phenyl group, and a tert-butyl group.

5. The organic compound according to claim 1, wherein n in the general formula [1] is 3 or 4.

6. An organic light-emitting device comprising:
a first electrode;
a second electrode; and
an organic compound layer between the first electrode and the second electrode,
wherein the organic compound layer contains the organic compound according to claim 1.

7. The organic light-emitting device according to claim 6, wherein
the organic compound layer includes a light-emitting layer, and
the light-emitting layer contains the organic compound.

8. The organic light-emitting device according to claim 7, wherein the light-emitting layer further contains a first compound, and
the organic compound has a lowest excited singlet energy higher than a lowest excited triplet energy of the first compound.

9. The organic light-emitting device according to claim 8, wherein the first compound has at least a tricyclic or polycyclic fused-ring structure.

10. The organic light-emitting device according to claim 9, wherein the first compound has a structure represented by one of the general formulae [Ir-1] to [Ir-12], 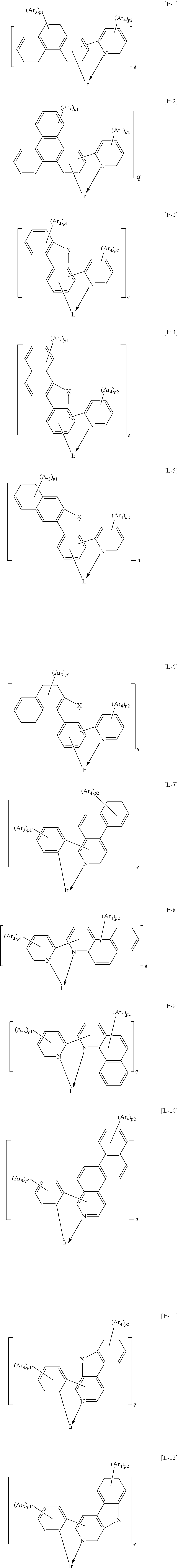
wherein Ar₁ and Ar₄ in the general formulae [Ir-1] to [Ir-12] denote a deuterium atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted silyl group, or a cyano group, X is selected from an oxygen atom, a sulfur atom, C(R₁) (R₂), and NR₃, R₁ to R₃ are each independently selected from a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted aryl group, a substituted or unsubstituted heterocyclic group, a substituted or unsubstituted silyl group, and a cyano group, p₁ and p₂ each denote an integer in the range of 0 to 4, and q denotes an integer in the range of 1 to 3.

11. The organic light-emitting device according to claim 10, wherein the first compound has any one of a triphenylene skeleton, a phenanthrene skeleton, a fluorene skeleton, a benzofluorene skeleton, a dibenzofuran skeleton, a dibenzothiophene skeleton, a benzoisoquinoline skeleton, and a naphthoisoquinoline skeleton.

12. The organic light-emitting device according to claim 8, wherein the light-emitting layer further contains a second compound, and
the second compound has a lowest excited singlet energy higher than a lowest excited singlet energy of the first compound.

13. The organic light-emitting device according to claim 12, wherein the second compound has at least one of a carbazole skeleton, an azine ring, and a xanthone skeleton.

14. A display apparatus comprising a plurality of pixels, wherein at least one of the plurality of pixels includes the organic light-emitting device according to claim 6 and a transistor coupled to the organic light-emitting device.

15. A photoelectric conversion apparatus comprising:
an optical unit including a plurality of lenses;
an imaging device configured to receive light passing through the optical unit; and
a display unit configured to display an image taken by the imaging device,
wherein the display unit includes the organic light-emitting device according to claim 6.

16. Electronic equipment comprising:
a display unit including the organic light-emitting device according to claim 6;
a housing configured to be provided with the display unit; and
a communication unit provided in the housing and configured to communicate with an outside.

17. A lighting apparatus comprising:
a light source including the organic light-emitting device according to claim 6; and
a light-diffusing unit or an optical film configured to transmit light emitted by the light source.

18. A moving body comprising:
a lamp including the organic light-emitting device according to claim 6; and
a body configured to be provided with the lamp.

19. An image-forming apparatus comprising:
a photosensitive member; and
an exposure light source configured to expose the photosensitive member to light,
wherein the exposure light source includes the organic light-emitting device according to claim 6.
